SEQUENCE LISTING (2 sequences)

SEQ ID NO: 1 - DNA, 20 bp, synthetic construct
  source 1..20: mol_type = other DNA
aatgatacggcgaccaccga

SEQ ID NO: 2 - DNA, 24 bp, synthetic construct
  source 1..24: mol_type = other DNA
caagcagaagacggcatacgagat

(19) United States
(12) Patent Application Publication
(10) Pub. No.: US 2023/0323449 A1
(43) Pub. Date: Oct. 12, 2023

(54) COMPOSITIONS AND METHODS FOR DETECTION OF VARIANTS

(71) Applicant: Twist Bioscience Corporation, South San Francisco, CA (US)

(72) Inventors: Richard GANTT, San Francisco, CA (US); Adriana ARNESON, Daly City, CA (US)

(21) Appl. No.: 18/066,953

(22) Filed: Dec. 15, 2022

Related U.S. Application Data
(60) Provisional application No. 63/291,146, filed on Dec. 17, 2021.

(51) Int. Cl. C12Q 1/6874; C12Q 1/6855

(52) U.S. Cl. CPC C12Q 1/6874; C12Q 1/6855; C12Q 2600/16; C12Q 2600/156

(57) ABSTRACT

Testing the many hypotheses from genomics and systems biology experiments demands accurate and cost-effective gene and genome synthesis. Here we describe a microchip-based technology for multiplex gene synthesis. Pools of thousands of ‘construction’ oligonucleotides and tagged complementary ‘selection’ oligonucleotides are synthesized on photo-programmable microfluidic chips, released, amplified and selected by hybridization to reduce synthesis errors ninefold. A one-step polymerase assembly multiplexing reaction assembles these into multiple genes. This technology enabled us to synthesize all 21 genes that encode the proteins of the Escherichia coli 30S ribosomal subunit, and to optimize their translation efficiency in vitro through alteration of codon bias. This is a significant step towards the synthesis of ribosomes in vitro and should have utility for synthetic biology in general.

### CROSS-REFERENCE

This application claims the benefit of U.S. Provisional Patent Application No. 63/291,146 filed on Dec. 17, 2021, which is incorporated by reference in its entirety.

### SEQUENCE LISTING

The instant application contains a Sequence Listing which has been submitted electronically in XML format and is hereby incorporated by reference in its entirety. Said XML copy, created on Feb. 22, 2023, is named 44854-847_201_SL.xml and is 2,811 bytes in size.

### BACKGROUND

Nucleic acid sequencing with high fidelity and low cost has a central role in biotechnology and medicine, and in basic biomedical research. While various methods are known for sequencing complex nucleic acid samples, these techniques often suffer from scalability, automation, speed, accuracy, and cost.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Provided herein are compositions and methods for uniquely labeling polynucleotides.

Provided herein are methods of sequencing comprising: ligating one or more polynucleotide adapters to a plurality of sample nucleic acids to generate a library of adapter-ligated sample polynucleotides, wherein at least some of the polynucleotide adapters comprise a first unique molecular identifier and a second unique molecular identifier; amplifying the library; sequencing the enriched library to generate a plurality of reads; organizing the reads based on the first unique molecular identifier and the second unique molecular identifier to distinguish between amplification errors and single nucleotide polymorphisms present in the sample nucleic acids, and wherein at least 80% of SNV variants are called at a level of at least 1%. Further provided herein are methods wherein the SNV variants are called with a minimum sequencing depth of 10,000×. Further provided herein are methods wherein at least 90% of SNV variants are called at a level of at least 1%. Further provided herein are methods wherein at least 95% of SNV variants are called at a level of at least 1%. Further provided herein are methods wherein at least 95% of SNV variants are called at a level of at least 0.5%. Further provided herein are methods wherein at least 95% of SNV variants are called at a level of at least 1% with a minimum sequencing depth of 10,000×. Further provided herein are methods wherein at least 95% of SNV variants are called at a level of at least 1% with a minimum sequencing depth of 20,000×. Further provided herein are methods wherein the first unique molecular identifier and the second unique molecular identifier are selected from a set of no more than 64 sequences. Further provided herein are methods wherein the first unique molecular identifier and the second unique molecular identifier are selected from a set of no more than 48 sequences. Further provided herein are methods wherein the first unique molecular identifier and the second unique molecular identifier are selected from a set of sequences having a Hamming distance of at least 2. Further provided herein are methods wherein the duplex efficiency comprises the number of duplex reads after sequencing after the duplex collapses divided by the total number of input reads. Further provided herein are methods wherein the polynucleotide adapter comprises a duplex efficiency of at least 4%. Further provided herein are methods wherein the polynucleotide adapter comprises a duplex efficiency of at least 6%. Further provided herein are methods wherein the method has a recall of at least 20% for single nucleotide polymorphisms present at least at 0.2% abundance in the sample nucleic acids. Further provided herein are methods wherein the single nucleotide polymorphisms are present at less than 1% abundance in the sample nucleic acids. Further provided herein are methods wherein the first unique molecular identifier or the second unique molecular identifier comprise the sequences of one or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAAC, GTGAGA, GTGTCA, and TGTGCC. Further provided herein are methods wherein first unique molecular identifier or the second unique molecular identifier comprise the sequences of 10 or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. Further provided are methods wherein the first unique molecular identifier or the second unique molecular identifier comprise the sequences of one or more of AAGGA, ACAAC, ATACG, CACTG, CATGA, CGATA, CGTGT, GCCAT, GCTGT, GTCAC, GTCGT, TACGA, TCCTA, TCGTG, TGTCG, and TTGGC. Further provided herein are methods wherein the first unique molecular identifier or the second unique molecular identifier comprise the sequences of one or more of AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC.

Provided herein are libraries of polynucleotide adapters comprising: at least two polynucleotide adapters, each comprising: a first strand, wherein the first strand comprises a first terminal adapter region, a first non-complementary region, a first yoke region, and a first unique molecular identifier; and a second strand, wherein the second strand comprises a second terminal adapter region, a second non-complementary region, a second yoke region, and a second unique molecular identifier; wherein the first yoke region and the second yoke region are complementary, wherein the first non-complementary region and the second non-complementary region are not complementary, and wherein the fraction of each of the polynucleotides in the library is 1-5%. Further provided herein are polynucleotide adapters wherein the fraction of each of the polynucleotides in the library is 1.5-4.5%. Further provided herein are polynucleotide adapters wherein the library comprises at least 8 different unique molecular identifiers. Further provided herein are polynucleotide adapters wherein the library comprises at least 16 different unique molecular identifiers. Further provided herein are polynucleotide adapters the library comprises at least 32 different unique molecular identifiers. Further provided herein are polynucleotide adapters the complementary first yoke region and second yoke region are each less than 15 bases in length. Further provided herein are polynucleotide adapters wherein the complementary first yoke region and second yoke region are each than 10 bases in length. Further provided herein are polynucleotide adapters the complementary first yoke region and second yoke region are each less than 6 bases in length. Further provided herein are polynucleotide adapters the first unique molecular identifier is 4-8 bases in length. Further provided herein are polynucleotide adapters the second unique molecular identifier is 4-8 bases in length. Further provided herein are polynucleotide adapters the first unique molecular identifier and the second unique molecular identifier are 5 or 6 bases in length. Further provided herein are polynucleotide adapters the first unique molecular identifier and the second unique molecular identifier are complementary. Further provided herein are polynucleotide adapters the first unique molecular identifier and the second unique molecular identifier are not complementary. Further provided herein are polynucleotide adapters wherein the first unique molecular identifier or the second unique molecular identifier comprise the sequences of one or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAAC, GTGAGA, GTGTCA, and TGTGCC. Further provided herein are polynucleotide adapters wherein the first unique molecular identifier or the second unique molecular identifier comprise the sequences of 10 or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. Further provided herein are polynucleotide adapters wherein the first unique molecular identifier or the second unique molecular identifier comprise the sequences of one or more of AAGGA, ACAAC, ATACG, CACTG, CATGA, CGATA, CGTGT, GCCAT, GCTGT, GTCAC, GTCGT, TACGA, TCCTA, TCGTG, TGTCG, and TTGGC. Further provided herein are polynucleotide adapters wherein the first unique molecular identifier or the second unique molecular identifier comprise the sequences of one or more of AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. Provided herein are polynucleotide adapters further comprising a sample nucleic acid. Further provided herein are polynucleotide adapters wherein the sample nucleic acid is DNA. Further provided herein are polynucleotide adapters wherein the sample nucleic acid is genomic DNA. Further provided herein are polynucleotide adapters wherein the genomic DNA is of human origin. Further provided herein are polynucleotide adapters wherein the first strand or the second strand further comprises at least one barcode. Further provided herein are polynucleotide adapters wherein the at least one barcode is at least 8 bases in length. Further provided herein are polynucleotide adapters wherein the at least one barcode is 8-12 bases in length. Further provided herein are polynucleotide adapters wherein the at least one barcode identifies the origin of the sample nucleic acid.

Provided herein are kits comprising the polynucleotide adapters described herein.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIG. 1A depicts a workflow for attachment of adapters comprising unique molecular identifiers (UMIs) to a polynucleotide to form an adapter-ligated polynucleotide.

FIG. 1B depicts a workflow for amplification of adapter-ligated polynucleotides to form a library for sequencing.

FIG. 1C depicts a workflow for synthesis of a polynucleotide adapter comprising a UMI.

FIG. 1D depicts a workflow for synthesis of a polynucleotide adapter comprising a UMI, wherein the method comprises PCR extension of one strand of the adapter.

FIG. 1E depicts a workflow for synthesis of a polynucleotide adapter comprising a UMI, wherein the method comprises PCR extension of one strand of the adapter, followed by restriction enzyme cleavage.

FIG. 1F depicts a workflow for synthesis of a polynucleotide adapter comprising a UMI, wherein the method comprises restriction enzyme cleavage.

FIG. 2A depicts a workflow for duplex sequencing analysis to identify variants. “*” indicates potential errors introduced by PCR or sequencing, and “+” indicates true variants.

FIG. 2B depicts a plot of mean target coverage for all reads, all marked up reads, and duplex reads generated using adapters comprising unique molecular identifiers.

FIG. 2C depicts a plot of the number of families vs. individual samples for “cs-families” (total groups of reads when reads with same start-stop and fragment size are collapsed same thing as regular mark duplicates), “ss-families” (total groups of reads when reads with the same start-stop, fragment size and UMI are collapsed collapsing just on UMI information), “ds-families” (total groups of reads with the same start-stop, fragment size, UMI and from two strands of same molecule are collapsed same as ss-families, but additionally collapsed when reads are identified to come from the same duplex molecule. However, if a pair from the other strand is not found, the read is not filtered out). 10pctTruQ and 1pctTruQ refers to tiers of the Tru-Q NGS DNA Reference Standard having multiple endogenous SNPs, insertions and deletions. The x-axis is labeled Samples from 1-38 (left to right): 1. R591-10 ng-10pctTruQ-16-24-32-39-rep1-C01; 2. R591-10 ng-10pctTruQ-16-24-32-39-rep1-C02; 3. R591-10 ng-10pctTruQ-16-24-32-39-rep1-C03; 4. R591-10 ng-10pctTruQ-16-24-32-39-rep1-C04; 5. R591-10 ng-10pctTruO-16-24-32-39-rep2-D01; 6. R591-10 ng-10pctTruQ-16-24-32-39-rep2-D02; 7. R591-10 ng-10pctTruO-16-24-32-39-rep2-D03; 8. R591-10 ng-10pctTruQ-16-24-32-39-rep2-D04; 9. R591-5-10-50 ng-10pctTruQ-32pairs-rep1-A05; 10. R591-5-10-50 ng-10pctTruQ-32pairs-rep1-C05; 11. R591-5-10-50 ng-10pctTruQ-32pairs-rep1-E05; 12. R591-5-10-50 ng-10pctTruQ-32pairs-rep2-B05; 13. R591-5-10-50 ng-10pctTruQ-32pairs-rep2-D05; 14. R591-5-10-50 ng-10pctTruQ-32pairs-rep2-F05; 15. R591-50 ng-10pctTruQ-16-24-32-39-rep1-E01; 16. R591-50 ng-10pctTruQ-16-24-32-39-rep1-E02; 17. R591-50 ng-10pctTruQ-16-24-32-39-rep1-E03; 18. R591-50 ng-10pctTruQ-16-24-32-39-rep1-E04; 19. R591-50 ng-10pctTruQ-16-24-32-39-rep2-F01; 20. R591-50 ng-10pctTruO-16-24-32-39-rep2-F02; 21. R591-50 ng-10pctTruQ-16-24-32-39-rep2-F03; 22. R591-50 ng-10pctTruO-16-24-32-39-rep2-F04; 23. R591-50 ng-1pctTruQ-16-24-32-39-rep1-G01; 24. R591-50 ng-1pctTruQ-16-24-32-39-rep1-G02; 25. R591-50 ng-1pctTruQ-16-24-32-39-rep1-G03; 26. R591-50 ng-1pctTruQ-16-24-32-39-rep1-G04; 27. R591-50 ng-1pctTruQ-16-24-32-39-rep2-H01; 28. R591-50 ng-1pctTruQ-16-24-32-39-rep2-H02; 29. R591-50 ng-1pctTruQ-16-24-32-39-rep2-H03; 30. R591-50 ng-1pctTruQ-16-24-32-39-rep2-H04; 31. R591-5 ng-10pctTruQ-16-24-32-39-rep1-A01; 32. R591-5 ng-10pctTruQ-16-24-32-39-rep1-A02; 33. R591-5 ng-10pctTruQ-16-24-32-39-rep1-A03; 34. R591-5 ng-10pctTruQ-16-24-32-39-rep1-A04; 35. R591-5 ng-10pctTruQ-16-24-32-39-rep2-B01; 36. R591-5 ng-10pctTruQ-16-24-32-39-rep2-B02; 37. R591-5 ng-10pctTruQ-16-24-32-39-rep2-B03; 38. R591-5 ng-10pctTruQ-16-24-32-39-rep2-B04.

FIG. 3A depicts a plot of the number of families vs. individual samples for cs-families, ss-families, and ds-families using 5 ng of sample. The x-axis is labeled Samples from 1-10 (left to right): 1. R591-5-10-50 ng-10pctTruQ-32pairs-rep1-A05; 2. R591-5-10-50 ng-10pctTruQ-32pairs-rep1-B05; 3. R591-5 ng-10pctTruQ-16-24-32-39-rep1-A01; 4. R591-5 ng-10pctTruQ-16-24-32-39-rep1-A02; 5. R591-5 ng-10pctTruQ-16-24-32-39-rep1-A03; 6. R591-5 ng-10pctTruQ-16-24-32-39-rep1-A04; 7. R591-5 ng-10pctTruQ-16-24-32-39-rep2-B01; 8. R591-5 ng-10pctTruQ-16-24-32-39-rep2-B02; 9. R591-5 ng-10pctTruQ-16-24-32-39-rep2-B03; 10. R591-5 ng-10pctTruQ-16-24-32-39-rep2-B04.

FIG. 3B depicts a plot of the number of families vs. individual samples for cs-families, ss-families, and ds-families using 10 ng of sample. The x-axis is labeled Samples from 1-10 (left to right): 1. R591-10 ng-10pctTruQ-16-24-32-39-rep1-C01; 2. R591-10 ng-10pctTruQ-16-24-32-39-rep1-C02; 3. R591-10 ng-10pctTruQ-16-24-32-39-rep1-C03; 4. R591-10 ng-10pctTruQ-16-24-32-39-rep1-C04; 5. R591-10 ng-10pctTruQ-16-24-32-39-rep2-D01; 6. R591-10 ng-10pctTruQ-16-24-32-39-rep2-D02; 7. R591-10 ng-10pctTruQ-16-24-32-39-rep2-D03; 8. R591-10 ng-10pctTruQ-16-24-32-39-rep2-D04; 9. R591-5-10-50 ng-10pctTruQ-32pairs-rep1-C05; 10. R591-5-10-50 ng-10pctTruQ-32pairs-rep2-D05.

FIG. 3C depicts a plot of the number of families vs. individual samples for cs-families, ss-families, and ds-families using 50 ng of sample. The x-axis is labeled Samples from 1-18 (left to right): R591-50 ng-10pctTruQ-32pairs-rep1-E05; 2. R591-50 ng-10pctTruQ-32pairs-rep2-F05; 3. R591-50 ng-10pctTruQ-16-24-32-39-rep1-E01; 4. R591-50 ng-10pctTruO-16-24-32-39-rep1-E02; 5. R591-50 ng-10pctTruQ-16-24-32-39-rep1-E03; 6. R591-50 ng-10pctTruQ-16-24-32-39-rep1-E04; 7. R591-50 ng-1pctTruQ-16-24-32-39-rep2-F01; 8. R591-50 ng-1pctTruQ-16-24-32-39-rep2-F02; 9. R591-50 ng-10pctTruQ-16-24-32-39-rep2-F03; 10. R591-50 ng-10pctTruQ-16-24-32-39-rep2-F04; 11. R591-50 ng-1pctTruQ-16-24-32-39-rep1-G01; 12. R591-50 ng-1pctTruO-16-24-32-39-rep1-G02; 13. R591-50 ng-1pctTruQ-16-24-32-39-rep1-G03; 14. R591-50 ng-1pctTruQ-16-24-32-39-rep1-G04; 15. R591-50 ng-1pctTruQ-16-24-32-39-rep2-H01; 16. R591-50 ng-1pctTruQ-16-24-32-39-rep2-H02; 17. R591-50 ng-1pctTruQ-16-24-32-39-rep2-H03; 18. R591-50 ng-1pctTruQ-16-24-32-39-rep2-H04.

FIG. 4A depicts a plot of the number of families vs. individual samples for cs-families, ss-families, and ds-families for 16 UMI pairs. The x-axis is labeled Samples from 1-8 (left to right): 1. R591-10 ng-10pctTruQ-16-24-32-39-rep1-C01; 2. R591-10 ng-10pctTruQ-16-24-32-39-rep2-D01; 3. R591-50 ng-10pctTruQ-16-24-32-39-rep1-E01; 4. R591-50 ng-10pctTruQ-16-24-32-39-rep2-F01; 5. R591-50 ng-1pctTruQ-16-24-32-39-rep1-G01; 6. R591-50 ng-1pctTruQ-16-24-32-39-rep2-H01; 7. R591-5 ng-10pctTruQ-16-24-32-39-rep1-A01; 8. R591-5 ng-10pctTruQ-16-24-32-39-rep2-B01.

FIG. 4B depicts a plot of the number of families vs. individual samples for cs-families, ss-families, and ds-families for 24 UMI pairs. The x-axis is labeled Samples from 1-8 (left to right): 1. R591-10 ng-10pctTruQ-16-24-32-39-rep1-C02; 2. R591-10 ng-10pctTruQ-16-24-32-39-rep2-D02; 3. R591-50 ng-10pctTruQ-16-24-32-39-rep1-E02; 4. R591-50 ng-10pctTruQ-16-24-32-39-rep2-F02; 5. R591-50 ng-1pctTruQ-16-24-32-39-rep1-G02; 6. R591-50 ng-1pctTruQ-16-24-32-39-rep2-H02; 7. R591-5 ng-10pctTruQ-16-24-32-39-rep1-A02; 8. R591-5 ng-10pctTruQ-16-24-32-39-rep2-B02.

FIG. 5A depicts a plot of the number of families vs. individual samples for cs-families, ss-families, and ds-families for 32 UMI pairs. The x-axis is labeled Samples from 1-14 (left to right): 1. R591-10 ng-10pctTruQ-16-24-32-39-rep1-C03; 2. R591-10 ng-10pctTruQ-16-24-32-39-rep2-D03; 3. R591-5-10-50 ng-10pctTruQ-32pairs-rep1A05; 4. R591-5-10-50 ng-10pctTruQ-32pairs-rep1-C05; 5. R591-5-10-50 ng-10pctTruQ-32pairs-rep1-E05; 6. R591-5-10-50 ng-10pctTruQ-32pairs-rep2-B05; 7. R591-5-10-50 ng-10pctTruQ-32pairs-rep2-D05; 8. R591-5-10-50 ng-10pctTruQ-32pairs-rep2-F05; 9. R591-50 ng-10pctTruQ-16-24-32-39-rep1-E03; 10. R591-50 ng-10pctTruQ-16-24-32-39-rep2-F03; 11. R591-50 ng-1pctTruQ-16-24-32-39-rep1-G03; 12. R591-50 ng-1pctTruQ-16-24-32-39-rep2-H03; 13. R591-5 ng-10pctTruQ-16-24-32-39-rep1-A03; 14. R591-5 ng-10pctTruQ-16-24-32-39-rep2-B03.

FIG. 5B depicts a plot of the number of families vs. individual samples for cs-families, ss-families, and ds-families for 39 UMI pairs. The x-axis is labeled Samples from 1-8 (left to right): 1. R591-10 ng-10pctTruQ-16-24-32-39-rep1-C04; 2. R591-10 ng-10pctTruQ-16-24-32-39-rep2-D04; 3. R591-50 ng-10pctTruQ-16-24-32-39-rep1-E04; 4. R591-50 ng-10pctTruQ-16-24-32-39-rep2-F04; 5. R591-50 ng-1pctTruQ-16-24-32-39-rep1-G04; 6. R591-50 ng-1pctTruQ-16-24-32-39-rep2-H04; 7. R591-5 ng-10pctTruQ-16-24-32-39-rep1-A04; 8. R591-5 ng-10pctTruQ-16-24-32-39-rep2-B04.

FIG. 6A depicts a plot of variant calling accuracy for four different genes using 5, 10, or 50 ng of mass input, and 16, 24, 32, or 39 UMI pairs.

FIG. 6B depicts a plot of variant calling accuracy for four different genes using 5, 10, or 50 ng of mass input, and 0, 40, 80, 120, or 160× mean target coverage.

FIG. 7A depicts a plot of variant calling accuracy for four different genes using 5, 10, or 50 ng of mass input, and 7000× or 8000× mean target coverage using all reads (no UMIs and no deduplication). Total coverage depth was 20,000×.

FIG. 7B depicts a plot of variant calling accuracy for four different genes using 5, 10, or 50 ng of mass input, and 7000× or 8000× mean target coverage using all reads (no UMIs and no deduplication). Total coverage depth was 40,000×.

FIG. 8A depicts a plot of variant calling accuracy for four different genes using 5, 10, or 50 ng of mass input, and 7000× or 8000× mean target coverage using duplex consensus reads. Total coverage depth was 20,000×.

FIG. 8B depicts a plot of variant calling accuracy for four different genes using 5, 10, or 50 ng of mass input, and 7000× or 8000× mean target coverage using duplex consensus reads. Total coverage depth was 40,000×.

FIG. 9 depicts a schematic for fragmenting a sample, end repair, A-tailing, ligating universal adapters, and adding barcodes to the adapters via PCR amplification to generate a sequencing library. Additional steps optionally include enrichment, additional rounds of amplification, and/or sequencing (not shown).

FIG. 10 depicts an image of a plate having 256 clusters, each cluster having 121 loci with polynucleotides extending therefrom.

FIG. 11A depicts a plot of polynucleotide representation (polynucleotide frequency versus abundance, as measured absorbance) across a plate from synthesis of 29,040 unique polynucleotides from 240 clusters, each cluster having 121 polynucleotides.

FIG. 11B depicts a plot of measurement of polynucleotide frequency versus abundance absorbance (as measured absorbance) across each individual cluster, with control clusters identified by a box.

FIG. 12 illustrates a computer system.

FIG. 13 is a block diagram illustrating an architecture of a computer system.

FIG. 14 is a diagram demonstrating a network configured to incorporate a plurality of computer systems, a plurality of cell phones and personal data assistants, and Network Attached Storage (NAS).

FIG. 15 is a block diagram of a multiprocessor computer system using a shared virtual address memory space.

FIG. 16 depicts a general workflow for 1) enzymatic or mechanical fragmentation, 2) end repair, 3) A-tailing, 4) universal adapter ligation (optionally with UMIs), and 4) UDI primer PCR amplification.

FIG. 17A depicts a graph of DNA fragment size distribution of a gDNA fragment library immediately after mechanical shearing, analyzed using a high sensitivity DNA assay.

FIG. 17B depicts a graph of DNA fragment size distribution of a cfDNA fragment library, as analyzed using a high sensitivity DNA assay.

FIG. 18A depicts an electropherogram generated by analysis of a gDNA library sample that was prepared as described. Note the single prominent peak at −400 bp.

FIG. 18B depicts an electropherogram generated by analysis of a cfDNA library sample that was prepared as described. Note the prominent peak at −320 bp and secondary peak at −480 bp.

FIG. 19A depicts polynucleotides from libraries prepared as described herein containing a 5 base pair unique molecular identifier and a 10 base pair unique dual index, as well as standard p5 and p7 adapters.

FIG. 19B depicts an in-silico workflow for processing and consensus of UMI data.

FIG. 20 depicts a graph of sensitivity in variant detection with (bottom) and without (top) UMI error correction.

### DETAILED DESCRIPTION

Described herein are compositions and methods for next generation sequencing. Further provided herein are polynucleotide adapters configured for use with a polynucleotide library (e.g., from a sample). Such libraries in some instances comprise genomic DNA, RNA, or other nucleic acid. Further described herein are adapters which comprise unique molecular identifiers (UMIs). UMIs in some instances provide for uniquely identification of individual members of a polynucleotide library, which enables molecular counting and identification of potential errors generated during preparation of a polynucleotide library prior to sequencing.

### Definitions

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the oligonucleotide or polynucleotide being known and chosen before the synthesis or assembly of the nucleic acid molecules.

The term nucleic acid encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. The length of polynucleotides, when provided, are described as the number of bases and abbreviated, such as nt (nucleotides), bp (bases), kb (kilobases), Mb (megabases) or Gb (gigabases).

Provided herein are methods and compositions for production of synthetic (i.e. de novo synthesized or chemically synthesizes) polynucleotides. The term oligonucleic acid, oligonucleotide, oligo, and polynucleotide are defined to be synonymous throughout. Libraries of synthesized polynucleotides described herein may comprise a plurality of polynucleotides collectively encoding for one or more genes or gene fragments. In some instances, the polynucleotide library comprises coding or non-coding sequences. In some instances, the polynucleotide library encodes for a plurality of cDNA sequences. Reference gene sequences from which the cDNA sequences are based may contain introns, whereas cDNA sequences exclude introns. Polynucleotides described herein may encode for genes or gene fragments from an organism. Exemplary organisms include, without limitation, prokaryotes (e.g., bacteria) and eukaryotes (e.g., mice, rabbits, humans, and non-human primates). In some instances, the polynucleotide library comprises one or more polynucleotides, each of the one or more polynucleotides encoding sequences for multiple exons. Each polynucleotide within a library described herein may encode a different sequence, i.e., non-identical sequence. In some instances, each polynucleotide within a library described herein comprises at least one portion that is complementary to sequence of another polynucleotide within the library. Polynucleotide sequences described herein may be, unless stated otherwise, comprise DNA or RNA. A polynucleotide library described herein may comprise at least 10, 20, 50, 100, 200, 500, 1,000, 2,000, 5,000, 10,000, 20,000, 30,000, 50,000, 100,000, 200,000, 500,000, 1,000,000, or more than 1,000,000 polynucleotides. A polynucleotide library described herein may have no more than 10, 20, 50, 100, 200, 500, 1,000, 2,000, 5,000, 10,000, 20,000, 30,000, 50,000, 100,000, 200,000, 500,000, or no more than 1,000,000 polynucleotides. A polynucleotide library described herein may comprise 10 to 500, 20 to 1000, 50 to 2000, 100 to 5000, 500 to 10,000, 1,000 to 5,000, 10,000 to 50,000, 100,000 to 500,000, or 50,000 to 1,000,000 polynucleotides. A polynucleotide library described herein may comprise about 370,000; 400,000; 500,000 or more different polynucleotides.

Unique Molecular Identifiers

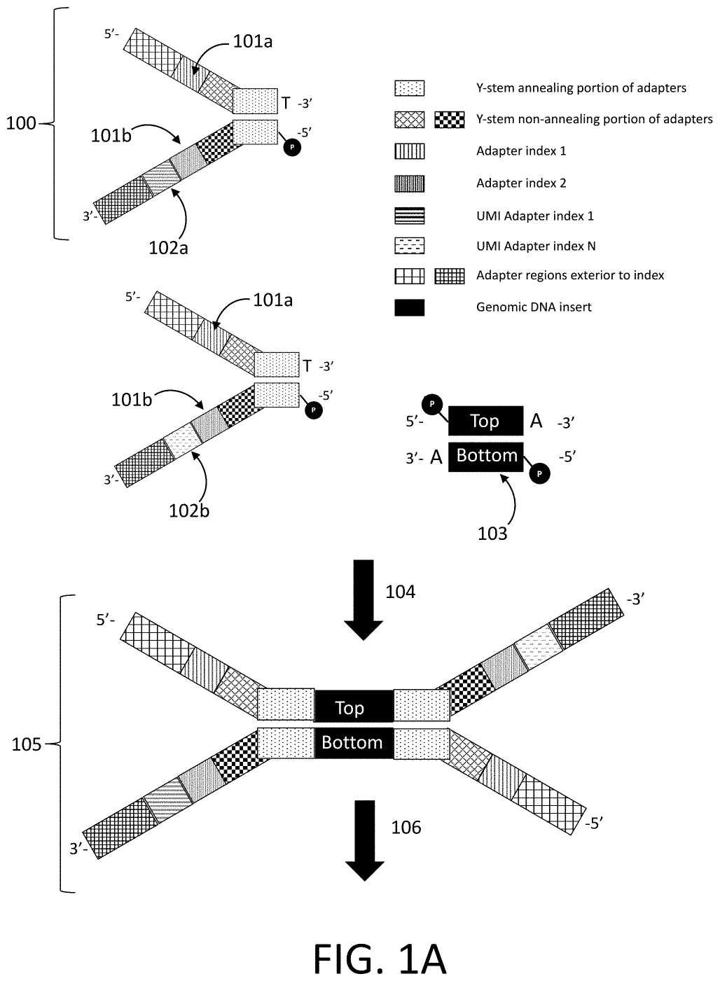

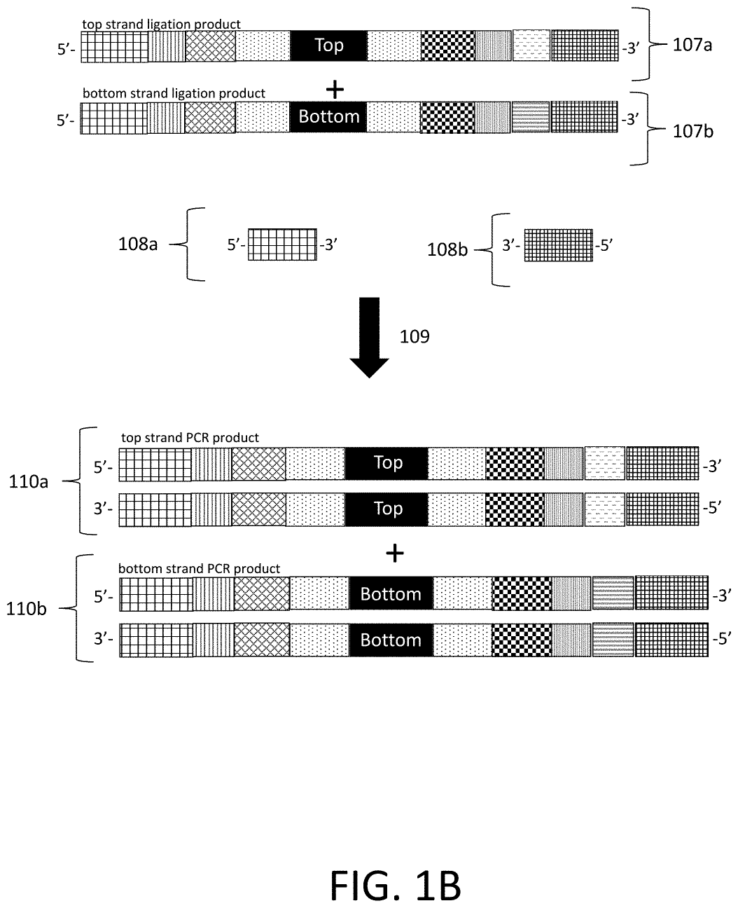

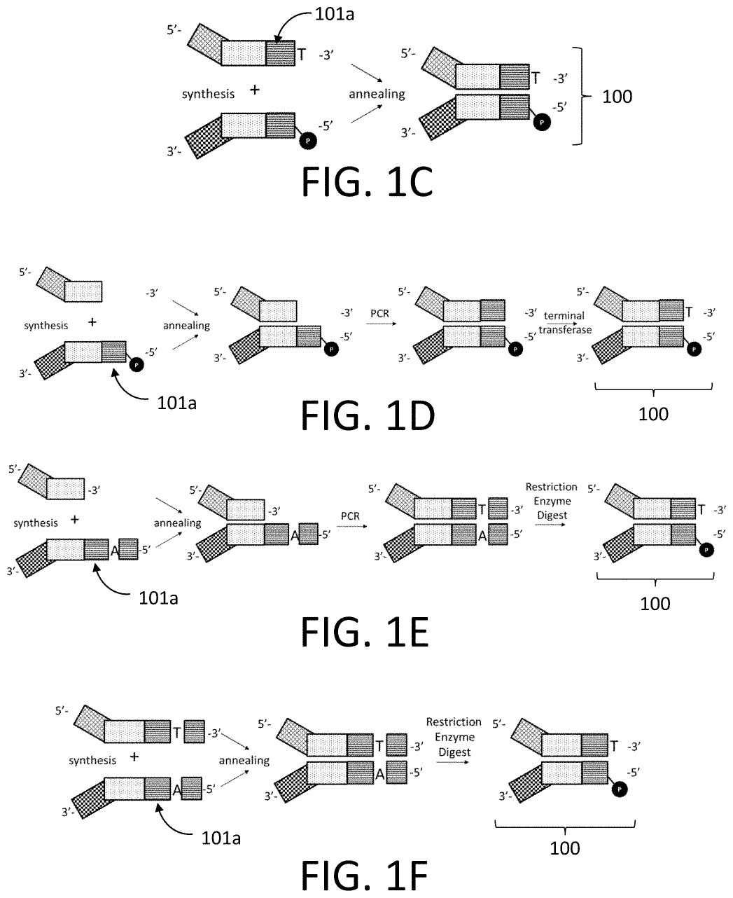

Described herein are adapters comprising unique molecular identifiers (UMIs). Adapters in some instances comprise a structure 100 of FIG. 1. In some instances, adapters comprise universal adapters. In some instances adapters comprise a Y-annealing region (anneals to form yoke), one or more Y-step non-annealing regions, a first index region 101a, a second index region 101b, a first UMI (index) region 102a, a second UMI (index) region 102b, and one or more regions exterior to the index. In some instances, adapters 100 are ligated 104 to sample polynucleotides 103 to form an adapter-ligated polynucleotide 105. After denaturation 106 of 105 (FIG. 1A), top 107a and bottom 107b strand ligation products are formed. In some instances, each strand is labeled with a different UMI. After amplification 109 with forward 108a and backward 108b primers, top strand 110a and bottom strand 110b PCR products are generated. In some instances, adapter ligated polynucleotides generated with universal adapters are further amplified with barcoded primers. In some instances adapters described herein comprise “in-line” UMIs, wherein at least one of a 5′ or 3′ UMI is not complementary to the other corresponding strand of the adapter (101a and 101b are not complementary). In some instances adapters described herein comprise “duplex” UMIs, wherein at least one of a 5′ or 3′ UMI is complementary to the other corresponding strand of the adapter (101a and 101b are complementary).

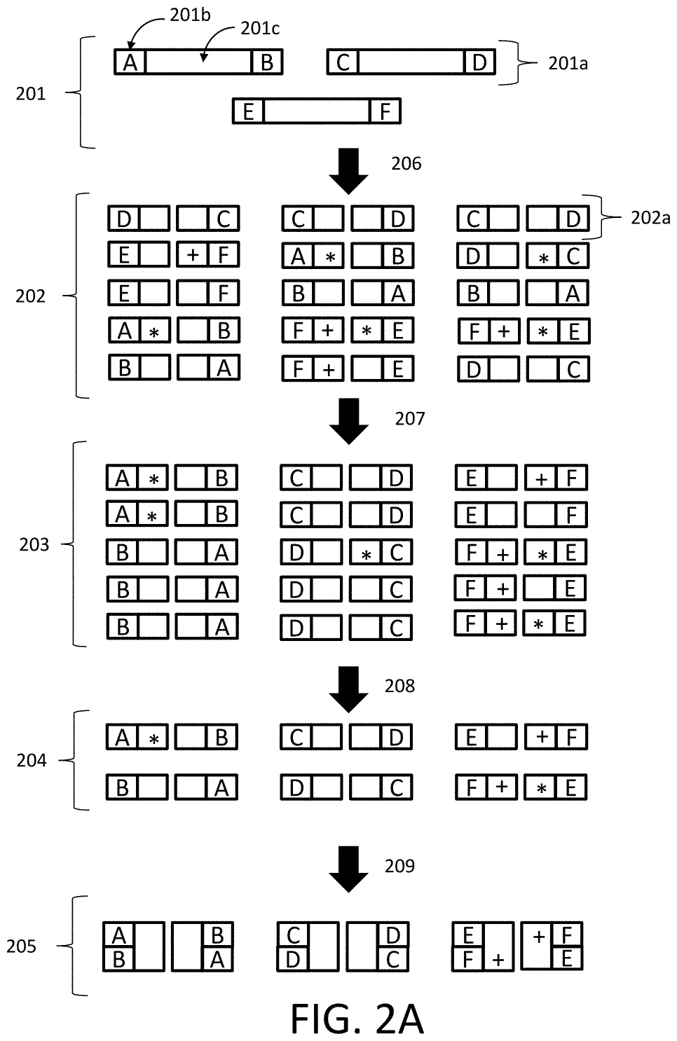

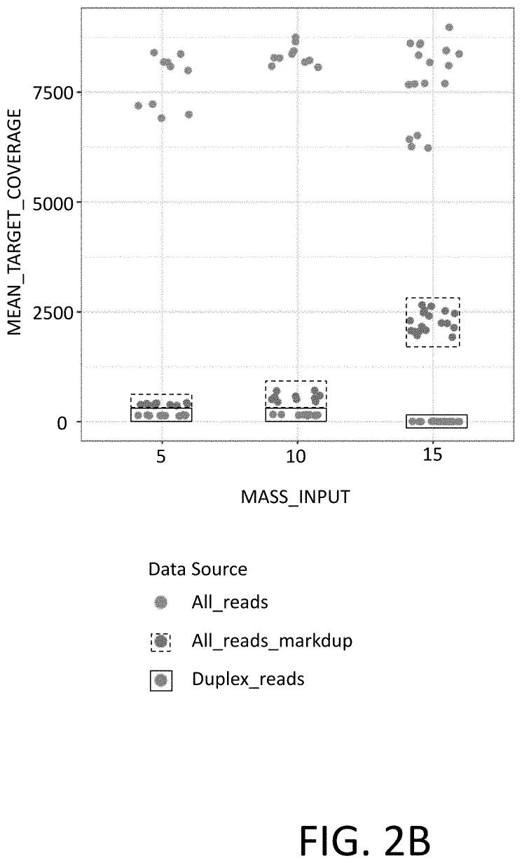

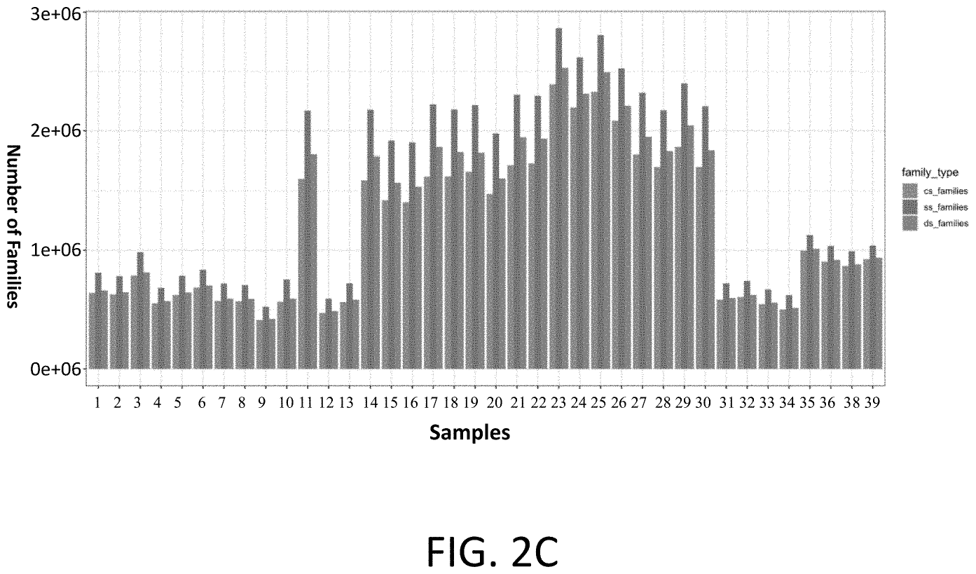

Adapter-ligated libraries comprising unique molecular identifiers may be used to distinguish between “true” mutations from a polynucleotide sample library and artifacts generated during sequencing library preparation (e.g., PCR errors, sequencing errors, or other erroneous base call). In some instances, a workflow as shown in FIG. 2A is used to analyze a library of adapter-ligated sample polynucleotides 201. Adapter-ligated sample polynucleotides 201 each comprise two distinct UMIs 201b represented by letters (A-F; six combinations of barcodes are shown for simplicity), and are attached to a sample polynucleotide 201c. After sequencing 206, forward and reverse read pairs 202 from sequencing are sorted into read pair groups 202a. Potential PCR-based errors are designated with “*”, and true polymorphisms are designated as “+”. Next, read pairs 203 are grouped 207 by barcode and barcode position. Single-stranded consensus sequences 204 are then generated 208 from each group of barcode-grouped read pairs. Errors from D-C, and F-E are identified, although the error in A-B remains. Finally, duplex consensus sequences 205 are generated 209 by comparing each set of single stranded consensus sequences. The error in A-B can be identified, and true mutation E-F can be confirmed. In some instances, errors include substitutions, deletions, or insertions. In some instances, an error is present in the sample polynucleotide portion of an adapter-ligated polynucleotide. In some instances, an error is present in a barcode configured to identify a sample origin (e.g., index) or to uniquely identify a sample polynucleotide. In some instances, an error is present in a UMI. In some instances, an error is present in a sample index. Compositions and methods described herein in some instances are used to identify such errors.

Described herein are sets of UMIs, wherein the set has defined properties. In some instances, a UMI set comprises a plurality of different polynucleotides having unique sequences. In some instances, a UMI set is 8, 12, 16, 20, 24, 30, 32, 36, 39, 48, or 64 unique sequences. In some instances, the sequences of a UMI set differ by a Hamming distance of no more than 1, 2, 3, 4, or 5. In some instances, the sequences of a UMI set differ by a Hamming distance of at least 1, 2, 3, 4, or 5. In some instances, the sequences of a UMI set differ by a Hamming distance of at least 2. In some instances, the sequences of a UMI set differ by a Hamming distance of at least 1.

UMIs may be any length, depending on the desired application. In some instances, a UMI is no more than 15, 12, 10, 8, 7, 6, 5, 4, or not more than 3 bases in length. In some instances, a UMI is about 15, 12, 10, 8, 7, 6, 5, 4, or about 3 bases in length. In some instances, a UMI is about 3-12, 3-10, 3-8. 4-12, 4-10, 4-8, 6-12, or 8-12 bases in length. UMIs in a set may comprise more than one length. In some instances, 10, 20, 25, 30, 40, 50, 60, or 70 percent of UMIs in the set are a first length, and 90, 80, 75, 70, 60, 50, 40, or 30 percent are a second length. In some instances, the first length is 3-5 bases, and the second length is 3-5 bases. In some instances, UMIs comprise lengths of 5 or 6 bases.

After addition of UMI-containing adapters to sample polynucleotides, at least some of the sample polynucleotides may be uniquely labeled. In some instances, at least 30%, 50%, 75%, 80%, 90%, 95%, or at least 98% of the sample polynucleotides are ligated to adapters comprising UMIs. In some instances, at least 1%, 2%, 5%, 10%, 15%, 20%, 30%, 50%, 75%, 80%, 90%, 95%, or at least 98% of the sample polynucleotides are labeled with a unique UMI sequence. In some instances, no more than 1%, 2%, 5%, 10%, 15%, 20%, 30%, 50%, 75%, 80%, 90%, 95%, or no more than 98% of the sample polynucleotides are labeled with a unique UMI sequence. In some instances, at least 1%, 2%, 5%, 10%, 15%, 20%, 30%, 50%, 75%, 80%, 90%, 95%, or at least 98% of the sample polynucleotides are uniquely identifiable after labeling with a UMI.

UMIs described herein in some instances comprise sequences of one or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. UMIs described herein in some instances comprise sequences of two or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. UMIs described herein in some instances comprise sequences of five or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. UMIs described herein in some instances comprise sequences of ten or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC.

UMIs may be represented at pre-selected percentages among a library of UMIs. In some instances at least 90% of the UMIs are present at fraction of 1-5%. In some instances at least 90% of the UMIs are present at fraction of 0.5%, 1%, 1.5%, 2%, 2.5%, 3%, 3.5%, 4%, 4.5%, 5%, 5.5%, 6%, 7%, or 8%. In some instances at least 90% of the UMIs are present at fraction of 0.5-8%, 1-7%, 1.5-7%, 2-7%, 2.5-6%, 3-8%, 3-6%, 1-5%, 0.5-5.5%, 1-4%, 1-6%, or 1-8%.

Any amount of sample polynucleotides (e.g., input DNA or other nucleic acid) may be ligated to adapters described herein. In some instances, the amount of sample polynucleotides is about 1, 5, 8, 10, 15, 20, 25, 30, 50, 75, or about 100 ng. In some instances, the amount of sample polynucleotides is no more than 1, 5, 8, 10, 15, 20, 25, 30, 50, 75, or no more than 100 ng. In some instances, the amount of sample polynucleotides is at least 1, 5, 8, 10, 15, 20, 25, 30, 50, 75, or at least 100 ng. In some instances, the amount of sample polynucleotides 1-10 ng, 1-100 ng, 3-10 ng, 5-100 ng, 5-75 ng, 5-50 ng, 10-100 ng, 10-50 ng, 25-100 ng, or 25-75 ng.

Provided herein are methods of generating adapters comprising UMIs. In a first method of adapter synthesis comprising synthesis of a top strand of an adapter comprising at least one UMI and a complementary bottom strand. After annealing the top and bottom adapter strands, an adapter comprising the structure of adapter 100 is formed (FIG. 1C). In a second method of adapter synthesis, a top strand is synthesized without a UMI, and a bottom strand comprising a complementary region and a UMI (FIG. 1D). After, annealing, PCR is used to generate a complementary UMI on the top strand, and a terminal transferase adds a T to the 3′ end of top strand to generate adapter 100. In a third method of synthesis, a top strand which does not comprise a UMI, and a bottom strand comprising a UMI, a restrictions site, and a 5′ overhang are synthesized (FIG. 1E). After annealing, the top strand is extended with PCR, and a restriction endonuclease is used to cleave a portion of the 3′ top strand and 5′ bottom strand to generate adapter 100. In a fourth method of adapter synthesis, two complementary strands each comprising a UMI, a restriction site, and an overhang portion (3′ top strand, 5′ bottom strand) are synthesized, annealed, and cleaved with a restriction enzyme to generate adapter 100. More than one UMIs may be present per adapter. In some instances, an adapter comprises 1, 2, 3, 4, 5, or more UMIs. In some instances, adapters comprise a first UMI and a second UMI. In some instances, a first UMI and a second UMI are complementary. In some instances, adapters comprise a first UMI and a second UMI. In some instances, a first UMI and a second UMI are not complementary. In some instances adapters are combined into libraries of adapters. In some instances adapters in a library comprise UMIs. In some instances adapters in a library comprise unique combinations of a first UMI and a second UMI.

Methylome Analysis

Analysis of the methylome may provide important information on biological processes for a given genomic sample. In some instances, methylated bases in a genomic sample are identified by either (a) conversion of a methylated base to a different base, or (b) conversion of a non-methylated base to a different base. Such conversions in some instances are performed on whole genomes or genomic fragments. The resulting sequences are then compared to a reference sequence (obtained without conversion/treatment) to identify which bases are methylated. In some instances, a conversion method (or process) comprises treatment with a deamination reagent. In some instances, a conversion method comprises treatment with bisulfite. In some instances, a conversion method comprises treatment with a reagent to protect methylcytosines (e.g., TET2 for oxidation), followed by treatment with an enzyme to deaminate unprotected cytosines (e.g., APOBEC). Additional reagents which differentiate methylated and non-methylated bases are also consistent with the methods disclosed herein. In some instances, unmethylated cytosines are converted to uracil. In some instances, PCR amplification of these uracil-containing modified genomes results in conversion of uracil to thymine. In some instances, adapters described herein are modified to replace cytosines with methylcytosines or other base which resists conversion.

Universal Adapters

Provided herein are universal adapters. In some instances, universal adapters comprise one or more unique molecular identifiers. In some instances, the universal adapters disclosed herein may comprise a universal polynucleotide adapter comprising a first strand and a second strand. In some instances, a first strand comprises a first primer binding region, a first non-complementary region, and a first yoke region. In some instances, a second strand comprises a second primer binding region, a second non-complementary region, and a second yoke region. In some instances, a primer binding region allows for PCR amplification of a polynucleotide adapter. In some instances, a primer binding region allows for PCR amplification of a polynucleotide adapter and concurrent addition of one or more barcodes to the polynucleotide adapter. In some instances, the first yoke region is complementary to the second yoke region. In some instances, the first non-complementary region is not complementary to the second non-complementary region. In some instances, the universal adapter is a Y-shaped or forked adapter. In some instances, one or more yoke regions comprise nucleobase analogues that raise the Tm between a first yoke region and a second yoke region. Primer binding regions as described herein may be in the form of a terminal adapter region of a polynucleotide. In some instances, a universal adapter comprises one index sequence. In some instances, a universal adapter comprises one unique molecular identifier. In some instances, universal adapters are configured for use with barcoded primers, wherein after ligation, barcoded primers are added via PCR.

A universal (polynucleotide) adapter may be shortened relative to a typical barcoded adapter (e.g., full-length “Y adapter”). For example, a universal adapter strand is 20-45 bases in length. In some instances, a universal adapter strand is 25-40 bases in length. In some instances, a universal adapter strand is 30-35 bases in length. In some instances, a universal adapter strand is no more than 50 bases in length, no more than 45 bases in length, no more than 40 bases in length, no more than 35 bases in length, no more than 30 bases in length, or no more than 25 bases in length. In some instances, a universal adapter strand is about 25, 27, 30, 32, 34, 36, 38, 40, 42, 44, 46, 48, 50, 52, 54, 56, 58, or about 60 bases in length. In some instances, a universal adapter strand is about 60 base pairs in length. In some instances, a universal adapter strand is about 58 base pairs in length. In some instances, a universal adapter strand is about 52 base pairs in length. In some instances, a universal adapter strand is about 33 base pairs in length.

A universal adapter may be modified to facilitate ligation with a sample polynucleotide. For example, the 5′ terminus is phosphorylated. In some instances, a universal adapter comprises one or more non-native nucleobase linkages such as a phosphorothioate linkage. For example, a universal adapter comprises a phosphorothioate between the 3′ terminal base, and the base adjacent to the 3′ terminal base. A sample polynucleotide in some instances comprises nucleic acid from a variety of sources, such as DNA or RNA of human, bacterial, plant, animal, fungal, or viral origin. An adapter-ligated sample polynucleotide in some instances comprises a sample polynucleotide (e.g., sample nucleic acid) with adapters universal adapters ligated to both the 5′ and 3′ end of the sample polynucleotide to form an adapter-ligated polynucleotide. A duplex sample polynucleotide comprises both a first strand (forward) and a second strand (reverse).

Universal adapters may contain any number of different nucleobases (DNA, RNA, etc.), nucleobase analogues, or non-nucleobase linkers or spacers. For example, an adapter comprises one or more nucleobase analogues or other groups that enhance hybridization (Tm) between two strands of the adapter. In some instances, nucleobase analogues are present in the yoke region of an adapter. Nucleobase analogues and other groups include but are not limited to locked nucleic acids (LNAs), bicyclic nucleic acids (BNAs), CS-modified pyrimidine bases, 2′-O-methyl substituted RNA, peptide nucleic acids (PNAs), glycol nucleic acid (GNAs), threose nucleic acid (TNAs), xenonucleic acids (XNAs) morpholino backbone-modified bases, minor grove binders (MGBs), spermine, G-clamps, or a anthraquinone (Uaq) caps. In some instances, adapters comprise one or more nucleobase analogues selected from Table 1.

TABLE 1

| Base | A | T | G | C | U |
|---|---|---|---|---|---|
| Locked Nucleic Acid (LNA) | 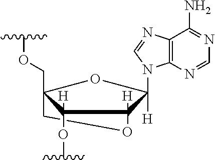 [SMILES: *OC[C@]12CO[C@@H](C1O*)[C@H](n1cnc3c(N)ncnc31)O2] | 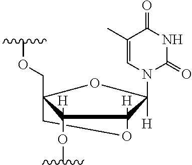 [SMILES: *OC[C@]12CO[C@@H](C1O*)[C@H](n1cc(C)c(=O)[nH]c1=O)O2] | 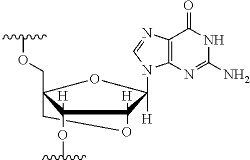 [SMILES: *OC[C@]12CO[C@@H](C1O*)[C@H](n1cnc3c(=O)[nH]c(N)nc31)O2] | 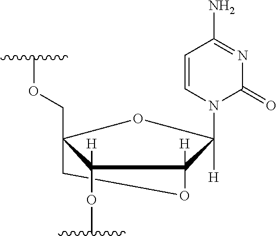 [SMILES: *OC[C@]12CO[C@@H](C1O*)[C@H](n1ccc(N)nc1=O)O2] | 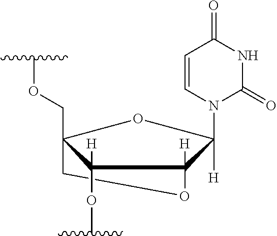 [SMILES: *OC[C@]12CO[C@@H](C1O*)[C@H](n1ccc(=O)[nH]c1=O)O2] |
| Bridged Nucleic Acid* (BNA) | 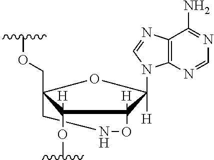 [SMILES: *OC[C@]12CNO[C@@H](C1O*)[C@H](n1cnc3c(N)ncnc31)O2] | 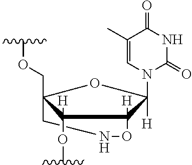 [SMILES: *OC[C@]12CNO[C@@H](C1O*)[C@H](n1cc(C)c(=O)[nH]c1=O)O2] | 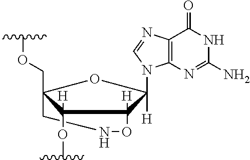 [SMILES: *OC[C@]12CNO[C@@H](C1O*)[C@H](n1cnc3c(=O)[nH]c(N)nc31)O2] | 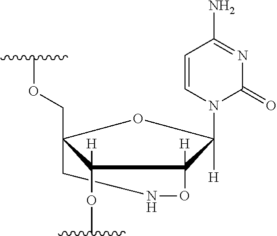 [SMILES: *OC[C@]12CNO[C@@H](C1O*)[C@H](n1ccc(N)nc1=O)O2] | 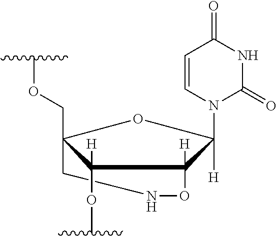 [SMILES: *OC[C@]12CNO[C@@H](C1O*)[C@H](n1ccc(=O)[nH]c1=O)O2] |

*R is H or Me.

Universal adapters may comprise any number of nucleobase analogues (such as LNAs or BNAs), depending on the desired hybridization Tm. For example, an adapter comprises 1 to 20 nucleobase analogues. In some instances, an adapter comprises 1 to 8 nucleobase analogues. In some instances, an adapter comprises at least 1, 2, 3, 4, 5, 6, 7, 8, 9, 10, 11, 12, or at least 12 nucleobase analogues. In some instances, an adapter comprises about 1, 2, 3, 4, 5, 6, 7, 8, 9, 10, 11, 12, 13, 14, 15, or about 16 nucleobase analogues. In some instances, the number of nucleobase analogous is expressed as a percent of the total bases in the adapter. For example, an adapter comprises at least 1%, 2%, 5%, 10%, 12%, 18%, 24%, 30%, or more than 30% nucleobase analogues. In some instances, adapters (e.g., universal adapters) described herein comprise methylated nucleobases, such as methylated cytosine.

### Barcodes

Polynucleotide primers may comprise defined sequences, such as barcodes (or indices). Adapters in some instances comprise one or more barcodes. In some instances, an adapter comprises at least one indexing barcode and at least one unique molecular identifier barcode. Barcodes can be attached to universal adapters, for example, using PCR and barcoded primers to generate barcoded adapter-ligated sample polynucleotides. Primer binding sites, such as universal primer binding sites, facilitate simultaneous amplification of all members of a barcode primer library, or a subpopulation of members. In some instances, a primer binding site comprises a region that binds to a flow cell or other solid support during next generation sequencing. In some instances, a barcoded primer comprises a P5 (5′-AATGATACGGCGACCACCGA-3′(SEQ ID NO: 1)) or P7 (5′-CAAGCAGAAGACGGCATACGAGAT-3′(SEQ ID NO: 2)) sequence. In some instances, primer binding sites are configured to bind to universal adapter sequences, and facilitate amplification and generation of barcoded adapters. In some instances, barcoded primers are no more than 60 bases in length. In some instances, barcoded primers are no more than 55 bases in length. In some instances, barcoded primers are 50-60 bases in length. In some instances, barcoded primers are about 60 bases in length. In some instances, barcodes described herein comprise methylated nucleobases, such as methylated cytosine.

The number of unique barcodes available for a barcode set (collection of unique barcodes or barcode combinations configured to be used together to unique define samples) may depend on the barcode length. In some instances, a Hamming distance is defined by the number of base differences between any two barcodes. In some instances, a Levenshtein distance is defined by the number changes needed to change one barcode into another (insertions, substitutions, or deletions). In some instances, barcode sets described herein comprise a Levenshtein distance of at least 2, 3, 4, 5, 6, 7, or at least 8. In some instances, barcode sets described herein comprise a Hamming distance of at least 2, 3, 4, 5, 6, 7, or at least 8.

Barcodes may be incorrectly associated with a different sample than they were assigned. In some instances, incorrect barcodes are occur from PCR errors (e.g., substitution) during library amplification. In some instances, entire barcodes “hop” or are transferred from one sample polynucleotide to another. Such transfers in some instances result from cross-contamination of free adapters or primers during a library generation workflow. In some instances a group of barcodes (barcode set) is chosen to minimize “barcode hopping”. In some instances, barcode hopping (for a single barcode) for a barcode set described herein is no more than 7%, 5%, 4%, 3%, 2%, 1%, 0.5%, or no more than 0.1%. In some instances, barcode hopping (for a single barcode) for a barcode set described herein is 0.1-6%, 0.1-5%, 0.2-5%, 0.5-5%, 1-7%, 1-5%, or 0.5-7%. In some instances, barcode hopping (for two barcodes) for a barcode set described herein is no more than 0.7%, 0.5%, 0.4%, 0.3%, 0.2%, 0.1%, 0.05%, or no more than 0.1%. In some instances, barcode hopping (for two barcodes) for a barcode set described herein is 0.01-0.6%, 0.01-0.5%, 0.02-0.5%, 0.05-0.5%, 0.1-0.7%, 0.1-0.5%, or 0.05-0.7%.

Barcoded primers comprise one or more barcodes. In some instances, the barcodes are added to universal adapters through PCR reaction. Barcodes are nucleic acid sequences that allow some feature of a polynucleotide with which the barcode is associated to be identified. In some instances, a barcode comprises an index sequence. In some instances, index sequences allow for identification of a sample, or unique source of nucleic acids to be sequenced. A barcode or combination of barcodes in some instances identifies a specific patient. A barcode or combination of barcodes in some instances identifies a specific sample from a patient among other samples from the same patient. After sequencing, the barcode (or barcode region) provides an indicator for identifying a characteristic associated with the coding region or sample source. Barcodes can be designed at suitable lengths to allow sufficient degree of identification, e.g., at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or more bases in length. Multiple barcodes, such as about 2, 3, 4, 5, 6, 7, 8, 9, 10, or more barcodes, may be used on the same molecule, optionally separated by non-barcode sequences. In some instances, a barcode is positioned on the 5′ and the 3′ sides of a sample polynucleotide. In some instances, each barcode in a plurality of barcodes differ from every other barcode in the plurality at least three base positions, such as at least about 3, 4, 5, 6, 7, 8, 9, 10, or more positions. Use of barcodes allows for the pooling and simultaneous processing of multiple libraries for downstream applications, such as sequencing (multiplex). In some instances, at least 4, 8, 16, 32, 48, 64, 128, or more 512 barcoded libraries are used. In some instances, at least 400, 500, 800, 1000, 2000, 5000, 10,000, 12,000, 15,000, 18,000, 20,000, or at 25,000 barcodes are used. Barcoded primers or adapters may comprise unique molecular identifiers (UMI). Such UMIs in some instances uniquely tag all nucleic acids in a sample. In some instances, at least 60%, 70%, 80%, 90%, 95%, or more than 95% of the nucleic acids in a sample are tagged with a UMI. In some instances, at least 85%, 90%, 95%, 97%, or at least 99% of the nucleic acids in a sample are tagged with a unique barcode, or UMI. Barcoded primers in some instances comprise an index sequence and one or more UMI. UMIs allow for internal measurement of initial sample concentrations or stoichiometry prior to downstream sample processing (e.g., PCR or enrichment steps) which can introduce bias. In some instances, UMIs comprise one or more barcode sequences. In some instances, each strand (forward vs. reverse) of an adapter-ligated sample polynucleotide possesses one or more unique barcodes. Such barcodes are optionally used to uniquely tag each strand of a sample polynucleotide. In some instances, a barcoded primer comprises an index barcode and a UMI barcode. In some instances, after amplification with at least two barcoded primers, the resulting amplicons comprise two index sequences and two UMIs. In some instances, after amplification with at least two barcoded primers, the resulting amplicons comprise two index barcodes and one UMI barcode. In some instances, each strand of a universal adapter-sample polynucleotide duplex is tagged with a unique barcode, such as a UMI or index barcode.

Barcoded primers in a library comprise a region that is complementary to a primer binding region on a universal adapter. For example, universal adapter binding region is complementary to primer region of the universal adapter, and universal adapter binding region is complementary to primer region of the universal adapter. Such arrangements facilitate extension of universal adapters during PCR, and attach barcoded primers. In some instances, the Tm between the primer and the primer binding region is 40-65 degrees C. In some instances, the Tm between the primer and the primer binding region is 42-63 degrees C. In some instances, the Tm between the primer and the primer binding region is 50-60 degrees C. In some instances, the Tm between the primer and the primer binding region is 53-62 degrees C. In some instances, the Tm between the primer and the primer binding region is 54-58 degrees C. In some instances, the Tm between the primer and the primer binding region is 40-57 degrees C. In some instances, the Tm between the primer and the primer binding region is 40-50 degrees C. In some instances, the Tm between the primer and the primer binding region is about 40, 45, 47, 50, 52, 53, 55, 57, 59, 61, or 62 degrees C.

Hybridization Blockers

Blockers may contain any number of different nucleobases (DNA, RNA, etc.), nucleobase analogues (non-canonical), or non-nucleobase linkers or spacers. In some instances, blockers comprise universal blockers. Such blockers may in some instances are described as a “set”, wherein the set comprises two or more blockers configured to prevent unwanted interactions with the same adapter sequence. In some instances, universal blockers prevent adapter-adapter interactions independent of one or more barcodes present on at least one of the adapters. For example, a blocker comprises one or more nucleobase analogues or other groups that enhance hybridization (Tm) between the blocker and the adapter. In some instances, a blocker comprises one or more nucleobases which decrease hybridization (Tm) between the blocker and the adapter (e.g., “universal” bases). In some instances, a blocker described herein comprises both one or more nucleobases which increase hybridization (Tm) between the blocker and the adapter and one or more nucleobases which decrease hybridization (Tm) between the blocker and the adapter.

Described herein are hybridization blockers comprising one or more regions which enhance binding to targeted sequences (e.g., adapter), and one or more regions which decrease binding to target sequences (e.g., adapter). In some instances, each region is tuned for a given desired level of off-bait activity during target enrichment applications. In some instances, each region can be altered with either a single type of chemical modification/moiety or multiple types to increase or decrease overall affinity of a molecule for a targeted sequence. In some instances, the melting temperature of all individual members of a blocker set are held above a specified temperature (e.g., with the addition of moieties such as LNAs and/or BNAs). In some instances, a given set of blockers will improve off bait performance independent of index length, independent of index sequence, and independent of how many adapter indices are present in hybridization.

Blockers may comprise moieties which increase and/or decrease affinity for a target sequencing, such as an adapter. In some instances, such specific regions can be thermodynamically tuned to specific melting temperatures to either avoid or increase the affinity for a particular targeted sequence. This combination of modifications is in some instances designed to help increase the affinity of the blocker molecule for specific and unique adapter sequence and decrease the affinity of the blocker molecule for repeated adapter sequence (e.g., Y-stem annealing portion of adapter). In some instances, blockers comprise moieties which decrease binding of a blocker to the Y-stem region of an adapter. In some instances, blockers comprise moieties which decrease binding of a blocker to the Y-stem region of an adapter, and moieties which increase binding of a blocker to non-Y-stem regions of an adapter.

Blockers (e.g., universal blockers) and adapters may form a number of different populations during hybridization. In a population ‘A’ in some instances comprises blockers correctly bound to non-index regions of the adapters. In a population ‘B’, a region of the blockers is bound to the “yoke” region of the adapter, but a remaining portion of the blocker does not bind to an adjacent region of the adapter. In a population ‘C’, two blockers unproductively dimerize. In a population ‘D’, blockers are unbound to any other nucleic acids. In some instances, when the number of DNA modifications that decrease affinity in the Y-stem annealing region of the blocker are increased, the populations ‘A’ & ‘D’ dominate and either have the desired or minimal effect. In some instances, as the number of DNA modifications that decrease affinity in the Y-stem annealing region of the blocker are decreased, the populations ‘B’ & ‘C’ dominate and have undesired effects where daisy-chaining or annealing to other adapters can occur (‘B’) or sequester blockers where they are unable to function properly (‘C’).

The index on both single or dual index adapter designs may be either partially or fully covered by universal blockers that have been extended with specifically designed DNA modifications to cover adapter index bases. In some instances, such modifications comprise moieties which decrease annealing to the index, such as universal bases. In some instances, the index of a dual index adapter is partially covered (or is overlapped) by one or more blockers. In some instances, the index of a dual index adapter is fully covered by one or more blockers. In some instances, the index of a single index adapter is partially covered by one or more blockers. In some instances, the index of a single index adapter is fully covered by one or more blockers. In some instances, a blocker overlaps an index sequence by at least 1, 2, 3, 4, 5, 6, 7, 8, 9, 10, 11, 12, 13, 14, 15, 20 or more than 20 bases. In some instances, a blocker overlaps an index sequence by no more than 1, 2, 3, 4, 5, 6, 7, 8, 9, 10, 11, 12, 13, 14, 15, 20, or no more than 25 bases. In some instances, a blocker overlaps an index sequence by about 1, 2, 3, 4, 5, 6, 7, 8, 9, 10, 11, 12, 13, 14, 15, 20 or about 30 bases. In some instances, a blocker overlaps an index sequence by 1-5, 1-3, 2-5, 2-8, 2-10, 3-6, 3-10, 4-10, 4-15, 1-4 or 5-7 bases. In some instances, a region of a blocker which overlaps an index sequences comprises at least one 2-deoxyinosine or 5-nitroindole nucleobase.

One or two blockers may overlap with an index sequence present on an adapter. In some instances, one or two blockers combined overlap with at least 1, 2, 3, 4, 5, 6, 7, 8, 9, 10, 11, 12, 13, 14, 15, 20 or more than 20 bases of the index sequence. In some instances, one or two blockers combined overlap with no more than 1, 2, 3, 4, 5, 6, 7, 8, 9, 10, 11, 12, 13, 14, 15, 20 or no more than 20 bases of the index sequence. In some instances, one or two blockers combined overlap with about 1, 2, 3, 4, 5, 6, 7, 8, 9, 10, 11, 12, 13, 14, 15, 20 or about 20 bases of the index sequence. In some instances, one or two blockers combined overlap by 1-5, 1-3, 2-5, 2-8, 2-10, 3-6, 3-10, 4-10, 4-15, 1-4 or 5-7 bases of the index sequence. In some instances, a region of a blocker which overlaps an index sequences comprises at least one 2-deoxyinosine or 5-nitroindole nucleobase.

In a first arrangement, the length of the adapter index overhang may be varied. When designed from a single side, the adapter index overhang can be altered to cover from 0 to n of the adapter index bases from either side of the index. This allows for the ability to design such adapter blockers for both single and dual index adapter systems.

In a second arrangement, the adapter index bases are covered from both sides. When adapter index bases are covered from both sides, the length of the covering region of each blocker can be chosen such that a single pair of blockers is capable of interacting with a range of adapter index lengths while still covering a significant portion of the total number of index bases. As an example, take two blockers that have been designed with 3 bp overhangs that cover the adapter index. In the context of 6 bp, 8 bp, or 10 bp adapter index lengths, these blockers will leave 0 bp, 2 bp, or 4 bp exposed during hybridization, respectively.

In a third arrangement, modified nucleobases are selected to cover index adapter bases. Examples of these modifications that are currently commercially available include degenerate bases (i.e., mixed bases of A, T, C, G), 2′-deoxyInosine, & 5-nitroindole.

In a forth arrangement, blockers with adapter index overhangs bind to either the sense (i.e., ‘top’) or anti-sense (i.e., ‘bottom’) strand of a next generation sequencing library.

In a fifth arrangement, blockers are further extended to cover other polynucleotide sequences (e.g., a poly-A tail added in a previous biochemical step in order to facilitate ligation or other method to introduce a defined adapter sequence, unique molecular identifier for bioinformatic assignment following sequencing, etc.) in addition to the standard adapter index bases of defined length and composition. These types of sequences can be placed in multiple locations of an adapter and in this case the most widely utilized case (i.e., unique molecular index next to the genomic insert) is presented. Other positions for the unique molecular identifier (e.g., next to adapter index bases) could also be addressed with similar approaches.

In a sixth arrangement, all of the previous arrangements are utilized in various combinations to meet a targeted performance metric for off-bait performance during target enrichment under specified conditions.

Blockers may comprise moieties, such as nucleobase analogues. Nucleobase analogues and other groups include but are not limited to locked nucleic acids (LNAs), bicyclic nucleic acids (BNAs), C5-modified pyrimidine bases, 2′-O-methyl substituted RNA, peptide nucleic acids (PNAs), glycol nucleic acid (GNAs), threose nucleic acid (TNAs), inosine, 2′-deoxyInosine, 3-nitropyrrole, 5-nitroindole, xenonucleic acids (XNAs) morpholino backbone-modified bases, minor grove binders (MGBs), spermine, G-clamps, or a anthraquinone (Uaq) caps. In some instances, nucleobase analogues comprise universal bases, wherein the nucleobase has a lower Tm for binding to a cognate nucleobase. In some instances, universal bases comprise 5-nitroindole or 2′-deoxyInosine. In instances, blockers comprise spacer elements that connect two polynucleotide chains. In some instances, blockers comprise one or more nucleobase analogues selected from Table 1. In some instances, such nucleobase analogues are added to control the Tm of a blocker. Blockers may comprise any number of nucleobase analogues (such as LNAs or BNAs), depending on the desired hybridization Tm. For example, a blocker comprises 20 to 40 nucleobase analogues. In some instances, a blocker comprises 8 to 16 nucleobase analogues. In some instances, a blocker comprises at least 1, 2, 3, 4, 5, 6, 7, 8, 9, 10, 11, 12, or at least 12 nucleobase analogues. In some instances, a blocker comprises about 1, 2, 3, 4, 5, 6, 7, 8, 9, 10, 11, 12, 13, 14, 15, or about 16 nucleobase analogues. In some instances, the number of nucleobase analogous is expressed as a percent of the total bases in the blocker. For example, a blocker comprises at least 1%, 2%, 5%, 10%, 12%, 18%, 24%, 30%, or more than 30% nucleobase analogues. In some instances, the blocker comprising a nucleobase analogue raises the Tm in a range of about 2° C. to about 8° C. for each nucleobase analogue. In some instances, the Tm is raised by at least or about 1° C., 2° C., 3° C., 4° C., 5° C., 6° C., 7° C., 8° C., 9° C., 10° C., 12° C., 14° C., or 16° C. for each nucleobase analogue. Such blockers in some instances are configured to bind to the top or “sense” strand of an adapter. Blockers in some instances are configured to bind to the bottom or “anti-sense” strand of an adapter. In some instances a set of blockers includes sequences which are configured to bind to both top and bottom strands of an adapter. Additional blockers in some instances are configured to the complement, reverse, forward, or reverse complement of an adapter sequence. In some instances, a set of blockers targeting a top (binding to the top) or bottom strand (or both) is designed and tested, followed by optimization, such as replacing a top blocker with a bottom blocker, or a bottom blocker with a top blocker. In some instances, a blocker is configured to overlap fully or partially with bases of an index or barcode on an adapter. A set of blockers in some instances comprise at least one blocker overlapping with an adapter index sequence. A set of blockers in some instances comprise at least one blocker overlapping with an adapter index sequence, and at least one blocker which does not overlap with an adapter sequence. A set of blockers in some instances comprise at least one blocker which does not overlap with a yoke region sequence. A set of blockers in some instances comprise at least one blocker which does not overlap with a yoke region sequence and at least one blocker which overlaps with a yoke region sequence. A sets of blockers in some instances comprises 2, 3, 4, 5, 6, 7, 8, 9, 10, or more than 10 blockers.

Blockers may be any length, depending on the size of the adapter or hybridization Tm. For example, blockers are 20 to 50 bases in length. In some instances, blockers are 25 to 45 bases, 30 to 40 bases, 20 to 40 bases, or 30 to 50 bases in length. In some instances, blockers are 25 to 35 bases in length. In some instances blockers are at least 25, 26, 27, 28, 29, 30, 31, 32, 33, 34, or at least 35 bases in length. In some instances, blockers are no more than 25, 26, 27, 28, 29, 30, 31, 32, 33, 34, or no more than 35 bases in length. In some instances, blockers are about 25, 26, 27, 28, 29, 30, 31, 32, 33, 34, or about 35 bases in length. In some instances, blockers are about 50 bases in length. A set of blockers targeting an adapter-tagged genomic library fragment in some instances comprises blockers of more than one length. Two blockers are in some instances tethered together with a linker. Various linkers are well known in the art, and in some instances comprise alkyl groups, polyether groups, amine groups, amide groups, or other chemical group. In some instances, linkers comprise individual linker units, which are connected together (or attached to blocker polynucleotides) through a backbone such as phosphate, thiophosphate, amide, or other backbone. In an exemplary arrangement, a linker spans the index region between a first blocker that each targets the 5′ end of the adapter sequence and a second blocker that targets the 3′ end of the adapter sequence. In some instances, capping groups are added to the 5′ or 3′ end of the blocker to prevent downstream amplification. Capping groups variously comprise polyethers, polyalcohols, alkanes, or other non-hybridizable group that prevents amplification. Such groups are in some instances connected through phosphate, thiophosphate, amide, or other backbone. In some instances, one or more blockers are used. In some instances, at least 4 non-identical blockers are used. In some instances, a first blocker spans a first 3′ end of an adaptor sequence, a second blocker spans a first 5′ end of an adaptor sequence, a third blocker spans a second 3′ end of an adaptor sequence, and a fourth blockers spans a second 5′ end of an adaptor sequence. In some instances a first blocker is at least 20, 21, 22, 23, 24, 25, 26, 27, 28, 29, 30, 31, 32, 33, 34, or at least 35 bases in length. In some instances a second blocker is at least 20, 21, 22, 23, 24, 25, 26, 27, 28, 29, 30, 31, 32, 33, 34, or at least 35 bases in length. In some instances a third blocker is at least 20, 21, 22, 23, 24, 25, 26, 27, 28, 29, 30, 31, 32, 33, 34, or at least 35 bases in length. In some instances a fourth blocker is at least 20, 21, 22, 23, 24, 25, 26, 27, 28, 29, 30, 31, 32, 33, 34, or at least 35 bases in length. In some instances, a first blocker, second blocker, third blocker, or fourth blocker comprises a nucleobase analogue. In some instances, the nucleobase analogue is LNA.

The design of blockers may be influenced by the desired hybridization Tm to the adapter sequence. In some instances, non-canonical nucleic acids (for example locked nucleic acids, bridged nucleic acids, or other non-canonical nucleic acid or analog) are inserted into blockers to increase or decrease the blocker's Tm. In some instances, the Tm of a blocker is calculated using a tool specific to calculating Tm for polynucleotides comprising a non-canonical amino acid. In some instances, a Tm is calculated using the Exiqon online prediction tool. In some instances, blocker Tm described herein are calculated in-silico. In some instances, the blocker Tm is calculated in-silico, and is correlated to experimental in-vitro conditions. Without being bound by theory, an experimentally determined Tm may be further influenced by experimental parameters such as salt concentration, temperature, presence of additives, or other factor. In some instances, Tm described herein are in-silico determined Tm that are used to design or optimize blocker performance. In some instances, Tm values are predicted, estimated, or determined from melting curve analysis experiments. In some instances, blockers have a Tm of 70 degrees C. to 99 degrees C. In some instances, blockers have a Tm of 75 degrees C. to 90 degrees C. In some instances, blockers have a Tm of at least 85 degrees C. In some instances, blockers have a Tm of at least 70, 72, 75, 77, 80, 82, 85, 88, 90, or at least 92 degrees C. In some instances, blockers have a Tm of about 70, 72, 75, 77, 80, 82, 85, 88, 90, 92, or about 95 degrees C. In some instances, blockers have a Tm of 78 degrees C. to 90 degrees C. In some instances, blockers have a Tm of 79 degrees C. to 90 degrees C. In some instances, blockers have a Tm of 80 degrees C. to 90 degrees C. In some instances, blockers have a Tm of 81 degrees C. to 90 degrees C. In some instances, blockers have a Tm of 82 degrees C. to 90 degrees C. In some instances, blockers have a Tm of 83 degrees C. to 90 degrees C. In some instances, blockers have a Tm of 84 degrees C. to 90 degrees C. In some instances, a set of blockers have an average Tm of 78 degrees C. to 90 degrees C. In some instances, a set of blockers have an average Tm of 80 degrees C. to 90 degrees C. In some instances, a set of blockers have an average Tm of at least 80 degrees C. In some instances, a set of blockers have an average Tm of at least 81 degrees C. In some instances, a set of blockers have an average Tm of at least 82 degrees C. In some instances, a set of blockers have an average Tm of at least 83 degrees C. In some instances, a set of blockers have an average Tm of at least 84 degrees C. In some instances, a set of blockers have an average Tm of at least 86 degrees C. Blocker Tm are in some instances modified as a result of other components described herein, such as use of a fast hybridization buffer and/or hybridization enhancer.

The molar ratio of blockers to adapter targets may influence the off-bait (and subsequently off-target) rates during hybridization. The more efficient a blocker is at binding to the target adapter, the less blocker is required. Blockers described herein in some instances achieve sequencing outcomes of no more than 20% off-target reads with a molar ratio of less than 20:1 (blocker:target). In some instances, no more than 20% off-target reads are achieved with a molar ratio of less than 10:1 (blocker:target). In some instances, no more than 20% off-target reads are achieved with a molar ratio of less than 5:1 (blocker:target). In some instances, no more than 20% off-target reads are achieved with a molar ratio of less than 2:1 (blocker:target). In some instances, no more than 20% off-target reads are achieved with a molar ratio of less than 1.5:1 (blocker:target). In some instances, no more than 20% off-target reads are achieved with a molar ratio of less than 1.2:1 (blocker:target). In some instances, no more than 20% off-target reads are achieved with a molar ratio of less than 1.05:1 (blocker:target).

The universal blockers may be used with panel libraries of varying size. In some embodiments, the panel libraries comprises at least or about 0.01, 0.02, 0.03, 0.04, 0.05, 0.06, 0.07, 0.08, 0.09, 1.0, 2.0, 4.0, 8.0, 10.0, 12.0, 14.0, 16.0, 18.0, 20.0, 22.0, 24.0, 26.0, 28.0, 30.0, 40.0, 50.0, 60.0, or more than 60.0 megabases (Mb).

Blockers as described herein may improve on-target performance. In some embodiments, on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95%. In some embodiments, the on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95% for various index designs. In some embodiments, the on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95% is improved for various panel sizes.

Hybridization Buffers

Any number of buffers may be used with the hybridization methods described herein. For example, a buffer comprises numerous chemical components, such as polymers, solvents, salts, surfactants, or other component. In some instances, hybridization buffers decrease the hybridization times (e.g., “fast” hybridization buffers) required to achieve a given sequencing result or level of quality. Such components in some instances lead to improved hybridization outcomes, such as increased on-target rate, improved sequencing outcomes (e.g., sequencing depth or other metric), or decreased off-target rates. Such components may be introduced at any concentration to achieve such outcomes. In some instances, buffer components are added in specific order. For example, water is added first. In some instances, salts are added after water. In some instances, salts are added after thickening agents and surfactants. In some instances, hybridization buffers such as “fast” hybridization buffers described herein are used in conjunction with universal blockers and liquid polymer additives. In some instances, use of fast hybridization buffers reduces hybridization times to no more than 4, 3, 2, 1, 0.5, 0.2, or 0.1 hours.

Hybridization buffers described herein may comprise solvents, or mixtures of two or more solvents. In some instances, a hybridization buffer comprises a mixture of two solvents, three solvents or more than three solvents. In some instances, a hybridization buffer comprises a mixture of an alcohol and water. In some instances, a hybridization buffer comprises a mixture of a ketone containing solvent and water. In some instances, a hybridization buffer comprises a mixture of an ethereal solvent and water. In some instances, a hybridization buffer comprises a mixture of a sulfoxide-containing solvent and water. In some instances, a hybridization buffer comprises a mixture of am amide-containing solvent and water. In some instances, a hybridization buffer comprises a mixture of an ester-containing solvent and water. In some instances, hybridization buffers comprise solvents such as water, ethanol, methanol, propanol, butanol, other alcohol solvent, or a mixture thereof. In some instances, hybridization buffers comprise solvents such as acetone, methyl ethyl ketone, 2-butanone, ethyl acetate, methyl acetate, tetrahydrofuran, diethyl ether, or a mixture thereof. In some instances, hybridization buffers comprise solvents such as DMSO, DMF, DMA, HMPA, or a mixture thereof. In some instances, hybridization buffers comprise a mixture of water, HMPA, and an alcohol. In some instances, two solvents are present at a 1:1, 1:2, 1:3, 1:4, 1:5, 1:8, 1:9, 1:10, 1:20, 1:50, 1:100, or 1:500 ratio.

Hybridization buffers described herein may comprise polymers. Polymers include but are not limited to thickening agents, polymeric solvents, dielectric materials, or other polymer. Polymers are in some instances hydrophobic or hydrophilic. In some instances, polymers are silicon polymers. In some instances, polymers comprise repeating polyethylene or polypropylene units, or a mixture thereof. In some instances, polymers comprise polyvinylpyrrolidone or polyvinylpyridine. In some instances, polymers comprise amino acids. For example, in some instances polymers comprise proteins. In some instances, polymers comprise casein, milk proteins, bovine serum albumin, or other protein. In some instances, polymers comprise nucleotides, for example, DNA or RNA. In some instances, polymers comprise polyA, polyT, Cot-1 DNA, or other nucleic acid. In some instances, polymers comprise sugars. For example, in some instances a polymer comprises glucose, arabinose, galactose, mannose, or other sugar. In some instances, a polymer comprises cellulose or starch. In some instances, a polymer comprises agar, carboxyalkyl cellulose, xanthan, guar gum, locust bean gum, gum karaya, gum tragacanth, gum Arabic. In some instances, a polymer comprises a derivative of cellulose or starch, or nitrocellulose, dextran, hydroxyethyl starch, ficoll, or a combination thereof. In some instances, mixtures of polymers are used in hybridization buffers described herein. In some instances, hybridization buffers comprise Denhardt's solution. Polymers described herein may be present at any concentration suitable for reducing off-target binding. Such concentrations are often represented as a percent by weight, percent by volume, or percent weight per volume. For example, a polymer is present at about 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or about 30%. In some instances, a polymer is present at no more than 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or no more than 30%. In some instances, a polymer is present in at least 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or at least 30%. In some instances, a polymer is present at 0.0001%-10%, 0.0002%-5%, 0.0005%-1.5%, 0.0008%-1%, 0.001%-0.2%, 0.002%-0.08%, 0.005%-0.02%, or 0.008%-0.05%. In some instances, a polymer is present at 0.005%-0.1%. In some instances, a polymer is present at 0.05%-0.1%. In some instances, a polymer is present at 0.005%-0.6%. In some instances, a polymer is present at 1%-30%, 5%-25%, 10%-30%, 15%-30%, or 1%-15%. Liquid polymers may be present as a percentage of the total reaction volume. In some instances, a polymer is about 10%, 20%, 30%, 40%, 50%, 60%, 75%, or about 90% of the total volume. In some instances, a polymer is at least 10%, 20%, 30%, 40%, 50%, 60%, 75%, or at least 90% of the total volume. In some instances, a polymer is no more than 10%, 20%, 30%, 40%, 50%, 60%, 75%, or no more than 90% of the total volume. In some instances, a polymer is 5%-75%, 5%-65%, 5%-55%, 10%-50%, 15%-40%, 20%-50%, 20%-30%, 25%-35%, 5%-35%, 10%-35%, or 20%-40% of the total volume. In some instances, a polymer is 25%-45% of the total volume. In some instances, hybridization buffers described herein are used in conjunction with universal blockers and liquid polymer additives.

Hybridization buffers described herein may comprise salts such as cations or anions. For example, hybridization buffer comprises a monovalent or divalent cation. In some instances, a hybridization buffer comprises a monovalent or divalent anion. Cations in some instances comprise sodium, potassium, magnesium, lithium, tris, or other salt. Anions in some instances comprise sulfate, bisulfite, hydrogensulfate, nitrate, chloride, bromide, citrate, ethylenediaminetetraacetate, dihydrogenphosphate, hydrogenphosphate, or phosphate. In some instances, hybridization buffers comprise salts comprising any combination of anions and cations (e.g. sodium chloride, sodium sulfate, potassium phosphate, or other salt). In some instance, a hybridization buffer comprises an ionic liquid. Salts described herein may be present at any concentration suitable for reducing off-target binding. Such concentrations are often represented as a percent by weight, percent by volume, or percent weight per volume. For example, a salt is present at about 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or about 30%. In some instances, a salt is present at no more than 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or no more than 30%. In some instances, a salt is present in at least 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or at least 30%. In some instances, a salt is present at 0.0001%-10%, 0.0002%-5%, 0.0005%-1.5%, 0.0008%-1%, 0.001%-0.2%, 0.002%-0.08%, 0.005%-0.02%, or 0.008%-0.05%. In some instances, a salt is present at 0.005%-0.1%. In some instances, a salt is present at 0.05%-0.1%. In some instances, a salt is present at 0.005%-0.6%. In some instances, a salt is present at 1%-30%, 5%-25%, 10%-30%, 15%-30%, or 1%-15%. Liquid polymers may be present as a percentage of the total reaction volume. In some instances, a salt is about 10%, 20%, 30%, 40%, 50%, 60%, 75%, or about 90% of the total volume. In some instances, a salt is at least 10%, 20%, 30%, 40%, 50%, 60%, 75%, or at least 90% of the total volume. In some instances, a salt is no more than 10%, 20%, 30%, 40%, 50%, 60%, 75%, or no more than 90% of the total volume. In some instances, a salt is 5%-75%, 5%-65%, 5%-55%, 10%-50%, 15%-40%, 20%-50%, 20%-30%, 25%-35%, 5%-35%, 10%-35%, or 20%-40% of the total volume. In some instances, a salt is 25%-45% of the total volume.

Hybridization buffers described herein may comprise surfactants (or emulsifiers). For example, a hybridization buffer comprises SDS (sodium dodecyl sulfate), CTAB, cetylpyridinium, benzalkonium tergitol, fatty acid sulfonates (e.g., sodium lauryl sulfate), ethyloxylated propylene glycol, lignin sulfonates, benzene sulfonate, lecithin, phospholipids, dialkyl sulfosuccinates (e.g., dioctyl sodium sulfosuccinate), glycerol diester, polyethoxylated octyl phenol, abietic acid, sorbitan monoester, perfluoro alkanols, sulfonated polystyrene, betaines, dimethyl polysiloxanes, or other surfactant. In some instances, a hybridization buffer comprises a sulfate, phosphate, or tetralkyl ammonium group. Surfactants described herein may be present at any concentration suitable for reducing off-target binding. Such concentrations are often represented as a percent by weight, percent by volume, or percent weight per volume. For example, a surfactant is present at about 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or about 30%. In some instances, a surfactant is present at no more than 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or no more than 30%. In some instances, a surfactant is present in at least 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or at least 30%. In some instances, a surfactant is present at 0.0001%-10%, 0.0002%-5%, 0.0005%-1.5%, 0.0008%-1%, 0.001%-0.2%, 0.002%-0.08%, 0.005%-0.02%, or 0.008%-0.05%. In some instances, a surfactant is present at 0.005%-0.1%. In some instances, a surfactant is present at 0.05%-0.1%. In some instances, a surfactant is present at 0.005%-0.6%. In some instances, a surfactant is present at 1%-30%, 5%-25%, 10%-30%, 15%-30%, or 1%-15%. Liquid polymers may be present as a percentage of the total reaction volume. In some instances, a surfactant is about 10%, 20%, 30%, 40%, 50%, 60%, 75%, or about 90% of the total volume. In some instances, a surfactant is at least 10%, 20%, 30%, 40%, 50%, 60%, 75%, or at least 90% of the total volume. In some instances, a surfactant is no more than 10%, 20%, 30%, 40%, 50%, 60%, 75%, or no more than 90% of the total volume. In some instances, a surfactant is 5%-75%, 5%-65%, 5%-55%, 10%-50%, 15%-40%, 20%-50%, 20%-30%, 25%-35%, 5%-35%, 10%-35%, or 20%-40% of the total volume. In some instances, a surfactant is 25%-45% of the total volume.

Buffers used in the methods described herein may comprise any combination of components. In some instances, a buffer described herein is a hybridization buffer. In some instances, a hybridization buffer described herein is a fast hybridization buffer. Such fast hybridization buffers allow for lower hybridization times such as less than 8 hours, 6 hours, 4 hours, 2 hours, 1 hour, 45 minutes, 30 minutes, or less than 15 minutes. Hybridization buffers described herein in some instances comprise a buffer described in Tables 2A-2G. In some instances, the buffers described in Tables 1A-1I may be used as fast hybridization buffers. In some instances, the buffers described in Tables 1B, 1C, and 1D may be used as fast hybridization buffers. In some instances, a fast hybridization buffer as described herein is described in Table 1B. In some instances, a fast hybridization buffer as described herein is described in Table 1C. In some instances, a fast hybridization buffer as described herein is described in Table 1D.

TABLE 2A
Buffers A

| | Volume | | Volume |
|---|---|---|---|
| Buffer Component | (mL) | Buffer Component | (mL) |
| Water | 5-300 | Water | 100-300 |
| DMF | 0-3 | DMSO | 0-3 |
| NaCl (5M) | 0.01-0.5 | NaCl (5M) | 0.01-0.5 |
| 20% SDS | 0.05-0.5 | 20% SDS | 0.05-0.5 |
| Tergitol (1% by weight) | 0.2-3 | EDTA (1M) | 0-2 |
| Denhardt's Solution (50×) | 1-10 | Denhardt's Solution (50×) | 1-10 |
| NaH₂PO₄ (5M) | 0.01-1.5 | NaH₂PO₄ (5M) | 0.01-1.5 |

TABLE 2B
Buffers B

| | Volume | | Volume |
|---|---|---|---|
| Buffer Component | (mL) | Buffer Component | (mL) |
| Water | 5-30 | Water | 5-30 |
| DMSO | 0.5-3 | DMSO | 0.5-3 |
| NaCl (5M) | 0.01-0.5 | NaCl (5M) | 0.01-0.5 |
| 20% SDS | 0.05-0.5 | 20% CTAB | 0.05-0.5 |
| EDTA (1M) | 0.05-2 | EDTA (1M) | 0.05-2 |
| Denhardt's Solution (50×) | 1-10 | Denhardt's Solution (50×) | 1-10 |
| NaH₂PO₄ (5M) | 0.01-1.5 | NaH₂PO₄ (5M) | 0.01-1.5 |

TABLE 2C
Buffers C

| | Volume | | Volume |
|---|---|---|---|
| Buffer Component | (mL) | Buffer Component | (mL) |
| Water | 5-30 | Water | 5-30 |
| DMSO | 0.5-3 | DMSO | 0.5-3 |
| NaCl (1M) | 0.01-0.5 | NaCl (5M) | 0.01-0.5 |
| 20% SDS | 0.05-0.5 | 20% SDS | 0.05-0.5 |
| TrisHCl (1M) | 0.01-2.5 | Dextran Sulfate (50%) | 0.05-2 |
| Denhardt's Solution (50×) | 1-10 | Denhardt's Solution (50×) | 1-10 |
| NaH₂PO₄ (5M) | 0.01-1.5 | NaH₂PO₄ (5M) | 0.01-1.5 |
| EDTA (0.5M) | 0.05-1.5 | EDTA (0.5M) | 0.05-1.5 |

TABLE 2D
Buffers D

| | Volume | | Volume |
|---|---|---|---|
| Buffer Component | (mL) | Buffer Component | (mL) |
| Water | 5-30 | Water | 5-30 |
| Methanol | 0.1-3 | DMSO | 0.5-3 |
| NaCl (1M) | 0.01-0.5 | NaCl (5M) | 0.01-0.5 |
| 20% Dextran Sulfate | 0.05-0.5 | 20% SDS | 0.05-0.5 |
| TrisHCl (1M) | 0.01-2.5 | hydroxyethyl starch (20%) | 0.05-2 |
| Denhardt's Solution (50×) | 1-10 | Denhardt's Solution (50×) | 1-10 |
| NaH₂PO₄ (1M) | 0.01-1.5 | NaH₂PO₄ (5M) | 0.01-1.5 |
| EDTA (0.5M) | 0.05-1.5 | EDTA (0.5M) | 0.05-1.5 |

TABLE 2E
Buffers E

| | Volume | | Volume |
|---|---|---|---|
| Buffer Component | (mL) | Buffer Component | (mL) |
| Water | 5-300 | Water | 5-300 |
| DMF | 0.1-30 | DMSO | 0.5-30 |
| NaCl (1M) | 0.01-0.5 | NaCl (5M) | 0.01-1.0 |
| hydroxyethyl starch (20%) | 0.01-2.5 | hydroxyethyl starch (20%) | 0.01-2.5 |
| Denhardt's Solution (50×) | 1-10 | Denhardt's Solution (50×) | 0.05-2 |
| NaH₂PO₄ (1M) | 0.01-1.5 | NaH₂PO₄ (5M) | 1-10 |

TABLE 2F
Buffers F

| | Volume | | Volume |
|---|---|---|---|
| Buffer Component | (mL) | Buffer Component | (mL) |
| Water | 50-300 | Water | 50-300 |
| DMF | 15-300 | DMSO | 15-300 |
| NaCl (5M) | 2-100 | NaCl (5M) | 2-100 |
| Denhardt's Solution (50×) | 1-10 | saline-sodium citrate 20× | 1-50 |
| Tergitol (1% by weight) | 0.2-2.0 | 20% SDS | 0-2 |

TABLE 2G
Buffers G

| | Volume | | Volume |
|---|---|---|---|
| Buffer Component | (mL) | Buffer Component | (mL) |
| Water | 5-30 | Water | 5-30 |
| Ethanol | 0-3 | Methanol | 0-3 |
| NaCl (1M) | 0.01-0.5 | NaCl (5M) | 0.01-0.5 |
| NaH₂PO₄ (5M) | 0.01-1.5 | NaH₂PO₄ (5M) | 0-2 |
| EDTA (0.5M) | 0-1.5 | EDTA (0.5M) | 1-10 |

TABLE 2H
Buffers H

| | Volume | | Volume |
|---|---|---|---|
| Buffer Component | (mL) | Buffer Component | (mL) |
| Water | 50-300 | Water | 10-300 |
| EDTA (0.5M) | 0-1.5 | NaCl (5M) | 0.01-0.5 |
| NaCl (5M) | 5-70 | 10% Triton X-100 | 0.05-0.5 |
| Tergitol (1% by weight) | 0.2-2.0 | EDTA (1M) | 0-2 |
| TrisHCl (1M) | 0.01-2.5 | TrisHCl (1M) | 0.1-5 |

TABLE 2I
Buffers I

| | Volume | | Volume |
|---|---|---|---|
| Buffer Component | (mL) | Buffer Component | (mL) |
| Water | 5-200 | Water | 10-200 |
| EDTA (0.5M) | 0-1.5 | NaCl (5M) | 0.01-0.5 |
| NaCl (5M) | 5-100 | Sodium Lauryl sulfate (10%) | 0.05-0.5 |
| CTAB (0.2M) | 0.05-0.5 | EDTA (1M) | 0-2 |

Buffers such as binding buffers and wash buffers are described herein. Binding buffers in some instances are used to prepare mixtures of sample polynucleotides and probes after hybridization. In some instances, binding buffers facilitate capture of sample polynucleotides on a column or other solid support. In some instances, the buffers described in Tables 2A-2I may be used as binding buffers. Binding buffers in some instances comprise a buffer described in Tables 2A, 2H, and 2I. In some instances, a binding buffer as described herein is described in Table 2A. In some instances, a binding buffer as described herein is described in Table 2H. In some instances, a binding buffer as described herein is described in Table 2I. In some instances, the buffers described herein may be used as wash buffers. Wash buffers in some instances are used to remove non-binding polynucleotides from a column or solid support. In some instances, the buffers described in Tables 2A-2I may be used as wash buffers. In some instances, a wash buffer comprises a buffer as described in Tables 2E, 2F, and 2G. In some instances, a wash buffer as described herein is described in Table 2E. In some instances, a wash buffer as described herein is described in Table 2F. In some instances, a wash buffer as described herein is described in Table 2G. Wash buffers used with the compositions and methods described herein are in some instances described as a first wash buffer (wash buffer 1), second wash buffer (wash buffer 2), etc.

Methods for Sequencing

Described herein are methods to improve the efficiency and accuracy of sequencing. Such methods comprise use of universal adapters comprising nucleobase analogues, and generation of barcoded adapters after ligation to sample nucleic acids. In some instances, a sample is fragmented, fragment ends are repaired, one or more adenines is added to one strand of a fragment duplex, universal adapters are ligated, and a library of fragments is amplified with barcoded primers to generate a barcoded nucleic acid library. Additional steps in some instances include enrichment/capture, additional PCR amplification, and/or sequencing of the nucleic acid library.

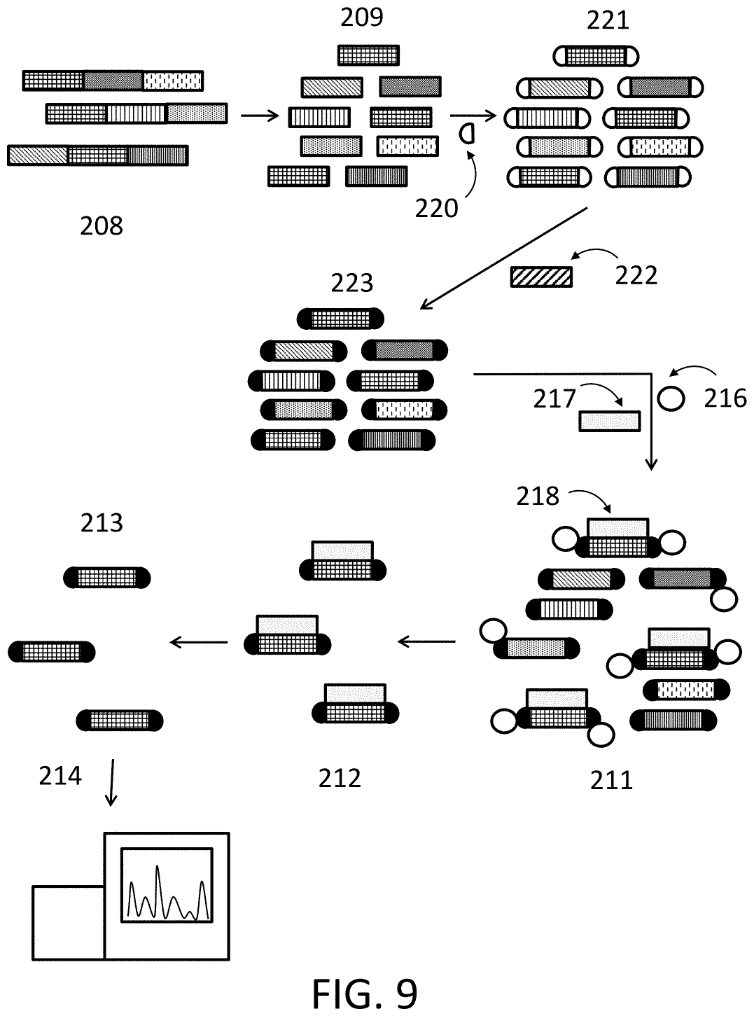

In a first step of an exemplary sequencing workflow (FIG. 9), a sample 208 comprising sample nucleic acids is fragmented by mechanical or enzymatic shearing to form a library of fragments 209. Universal adapters 220 are ligated to fragmented sample nucleic acids to form an adapter-ligated sample nucleic acid library 221. This library is then amplified with a barcoded primer library 222 (only one primer shown for simplicity) to generate a barcoded adapter-sample polynucleotide library 223. The library 223 is then optionally hybridized with target binding polynucleotides 217, which hybridize to sample nucleic acids, along with blocking polynucleotides 216 that prevent hybridization between probe polynucleotides 217 and adapters 220. Capture of sample polynucleotide-target binding polynucleotide hybridization pairs 212/218, and removal of target binding polynucleotides 217 allows isolation/enrichment of sample nucleic acids 213, which are then optionally amplified and sequenced 214. Various combinations of universal adapters and barcoded primers may be used. In some instances, barcoded primers comprise at least one barcode. In some instances, different types of barcodes are added to the sample nucleic acid using adapters or barcodes, or both. For example, a universal adapter comprises an index barcode, and after ligation is amplified with a barcoded primer comprising an additional index barcode. In some instances, a universal adapter comprises a unique molecular identifier barcode, and after ligation is amplified with a barcoded primer comprising an index barcode.

Barcoded primers may be used to amplify universal adapter-ligated sample polynucleotides using PCR, to generate a polynucleic acid library for sequencing. Such a library comprises barcodes after amplification in some instances. In some instances, amplification with barcoded primers results in higher amplification yields relative to amplification of a standard Y adapter-ligated sample polynucleotide library. In some instances, 2, 3, 4, 5, 6, 7, 8, 9, 10, 11, or 12 PCR cycles are used to amplify a universal adapter-ligated sample polynucleotide library. In some instances, no more than 2, 3, 4, 5, 6, 7, 8, 9, 10, 11, or no more than 12 PCR cycles are used to amplify a universal adapter-ligated sample polynucleotide library. In some instances, 2-12, 3-10, 4-9, 5-8, 6-10, or 8-12 PCR cycles are used to amplify a universal adapter-ligated sample polynucleotide library, thus generating amplicon products. Such libraries in some instances comprise fewer PCR-based errors. Without being bound by theory, reduced PCR cycles during amplification leads to fewer errors in resulting amplicon products. After amplification, such barcoded amplicon libraries are in some instances enriched or subjected to capture, additional amplification reactions, and/or sequencing. In some instances, amplicon products generated using the universal adapters described herein comprise about 30%, 15%, 10%, 7%, 5%, 3%, 2%, 1.5%, 1%, 0.5%, 0.1%, or 0.05% fewer errors than amplicon products generated from amplification of standard full-length Y adapters.

Described herein are methods wherein universal blockers are used to prevent off-target binding of capture probes to adapters ligated to genomic fragments, or adapter-adapter hybridization. Adapter blockers used for preventing off-target hybridization may target a portion or the entire adapter. In some instances, specific blockers are used that are complementary to a portion of the adapter that includes the unique index sequence. In cases where the adapter-tagged genomic library comprises a large number of different indices, it can be beneficial to design blockers which either do not target the index sequence, or do not hybridize strongly to it. For example, a “universal” blocker targets a portion of the adapter that does not comprise an index sequence (index independent), which allows a minimum number of blockers to be used regardless of the number of different index sequences employed. In some instances, no more than 8 universal blockers are used. In some instances, 4 universal blockers are used. In some instances, 3 universal blockers are used. In some instances, 2 universal blockers are used. In some instances, 1 universal blocker is used. In an exemplary arrangement, 4 universal blockers are used with adapters comprising at least 4, 8, 16, 32, 64, 96, or at least 128 different index sequences. In some instances, the different index sequences comprises at least or about 4, 6, 8, 10, 12, 14, 16, 18, 20, or more than 20 base pairs (bp). In some instances, a universal blocker is not configured to bind to a barcode sequence. In some instances, a universal blocker partially binds to a barcode sequence. In some instances, a universal blocker which partially binds to a barcode sequence further comprises nucleotide analogs, such as those that increase the Tm of binding to the adapter (e.g., LNAs or BNAs).

Provided herein are methods of using UMIs described herein. Further provided herein are methods of sequencing nucleic acids with UMIs. In some instances, a method comprises one or more steps of ligating one or more polynucleotide adapters to a plurality of sample nucleic acids to generate a library of adapter-ligated sample polynucleotides; amplifying the library; sequencing the enriched library to generate a plurality of reads; and organizing the reads based on the unique molecular identifier to distinguish between amplification errors and single nucleotide polymorphisms present in the sample nucleic acids. In some instances, a method comprises one or more steps of ligating one or more polynucleotide adapters to a plurality of sample nucleic acids to generate a library of adapter-ligated sample polynucleotides, wherein at least some of the polynucleotide adapters comprise a unique molecular identifier; amplifying the library; sequencing the enriched library to generate a plurality of reads; organizing the reads based on the unique molecular identifier to distinguish between amplification errors and single nucleotide polymorphisms present in the sample nucleic acids. In some instances, the polynucleotide adapters comprise a first unique molecular identifier and a second unique molecular identifier. Adapters comprising UMIs described herein and methods of use in sequencing thereof may increase various metrics of sequencing efficiency. In some instances, UMIs increase accuracy of recall of base calling. In some instances, UMIs described herein comprise increased duplex efficiency. In some instances, duplex efficiency comprises the number of duplex reads after sequencing after the duplex collapses divided by the total number of input reads. In some instances, adapters comprising UMIs comprise a duplex efficiency of at least 1%, 2%, 3%, 4%, 5%, 6%, 7%, 8%, or more. In some instances, adapters comprising UMIs comprise a duplex efficiency of 1%-5%, 1-10%, 1-8%, 2-6%, 2-10%, 3-5%, 3-8%, or 4-10%. In some instances, a method of using UMIs comprising adapters results in a recall of at least 20% for single nucleotide polymorphisms present at least at 0.2% abundance in the sample nucleic acids. In some instances, a method of using UMIs comprising adapters results in a recall of at least 20% for single nucleotide polymorphisms present at least at 0.5% abundance in the sample nucleic acids. In some instances, a method of using UMIs comprising adapters results in a recall of at least 20% for single nucleotide polymorphisms present at least at 1% abundance in the sample nucleic acids.

Adapters comprising UMIs described herein and methods of use thereof may increase efficiency of SNV calling. In some instances at least 80% of SNV variants are called at a level of at least 0.5%. In some instances at least 80% of SNV variants are called at a level of at least 1%. In some instances at least 80% of SNV variants are called at a level of at least 2%. In some instances at least 80% of SNV variants are called at a level of at least 1.5%. In some instances at least 90% of SNV variants are called at a level of at least 1%. In some instances at least 95% of SNV variants are called at a level of at least 1%. In some instances at least 95% of SNV variants are called at a level of at least 0.5%. In some instances at least 80% of SNV variants are called at a level of at least 0.5% with a minimum sequencing depth of 10,000×. In some instances at least 80% of SNV variants are called at a level of at least 1% with a minimum sequencing depth of 10,000×. In some instances at least 80% of SNV variants are called at a level of at least 2% with a minimum sequencing depth of 10,000×. In some instances at least 80% of SNV variants are called at a level of at least 1.5% with a minimum sequencing depth of 10,000×. In some instances at least 90% of SNV variants are called at a level of at least 1% with a minimum sequencing depth of 10,000×. In some instances at least 95% of SNV variants are called at a level of at least 1% with a minimum sequencing depth of 10,000×. In some instances at least 95% of SNV variants are called at a level of at least 0.5% with a minimum sequencing depth of 10,000×. In some instances at least 80% of SNV variants are called at a level of at least 0.5% with a minimum sequencing depth of 20,000×. In some instances at least 80% of SNV variants are called at a level of at least 1% with a minimum sequencing depth of 20,000×. In some instances at least 80% of SNV variants are called at a level of at least 2% with a minimum sequencing depth of 20,000×. In some instances at least 80% of SNV variants are called at a level of at least 1.5% with a minimum sequencing depth of 20,000×. In some instances at least 90% of SNV variants are called at a level of at least 1% with a minimum sequencing depth of 20,000×. In some instances at least 95% of SNV variants are called at a level of at least 1% with a minimum sequencing depth of 20,000×. In some instances at least 95% of SNV variants are called at a level of at least 0.5% with a minimum sequencing depth of 20,000×.

Methylation Sequencing and Capture

Methylation sequencing involves enzymatic or chemical methods leading to the conversion of unmethylated cytosines to uracil through a series of events culminating in deamination, while leaving methylated cytosines intact. During amplification, uracils are paired with adenines on the complementary strand, leading to the inclusion of thymine in the original position of the unmethylated cytosine. There are identical sequences with each having unmethylated-cytosines in different positions. The end product is asymmetric, yielding two different double stranded DNA molecules after conversion; the same process for methylated DNA leads to yet additional sets of sequences.

Target enrichment can proceed by pre- or post-capture conversion. Post-capture conversion targets the original sample DNA, while pre-capture targets the four strands of converted sequences. While post-capture conversion presents fewer challenges for probe design, it often requires large quantities of starting DNA material as PCR amplification does not preserve methylation patterns and cannot be performed before capture. Therefore, pre-capture conversion is often the method of choice for low-input, sensitive applications such as cell free DNA.

Methods described herein may comprise treatment of a library with enzymes or bisulfite to facilitate conversion of cytosines to uracil. In some instances, adapters (e.g., universal adapters) described herein comprise methylated nucleobases, such as methylated cytosine.

Kits

Provided herein are kits comprising polynucleotide adapters. In some instances, polynucleotide adapters comprise unique molecular identifiers as provided herein. The kits in some instances comprise packaging, instructions, standards, buffers or enzymes, or other reagents. In some instances, kits comprise probe libraries for enrichment capture as described herein.

De Novo Synthesis of Small Polynucleotide Populations for Amplification Reactions

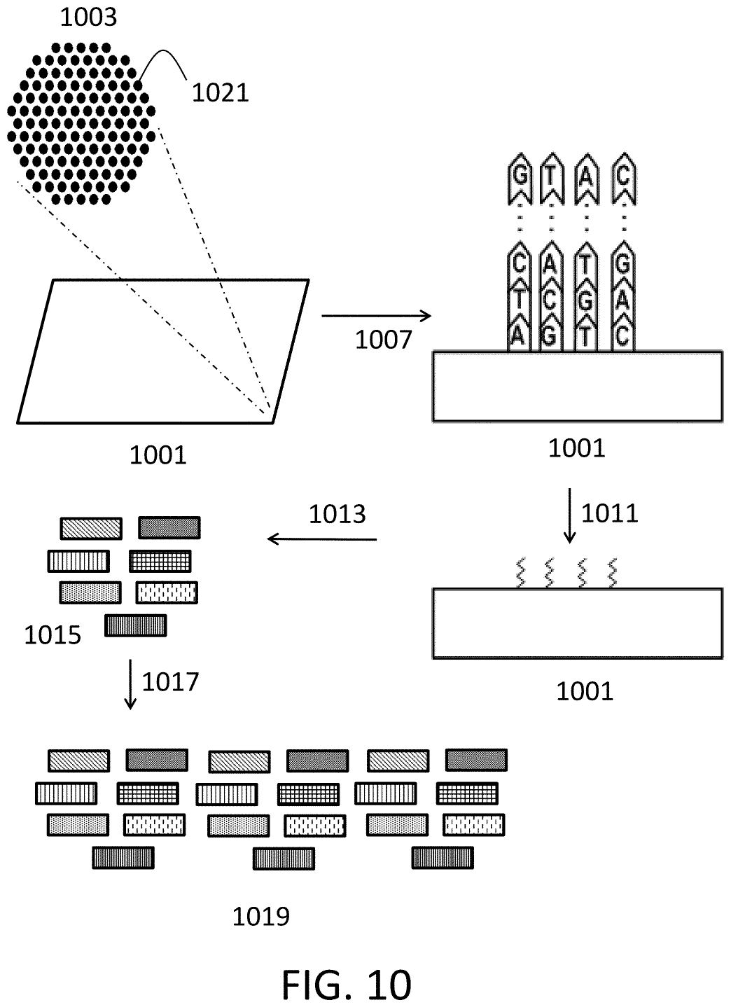

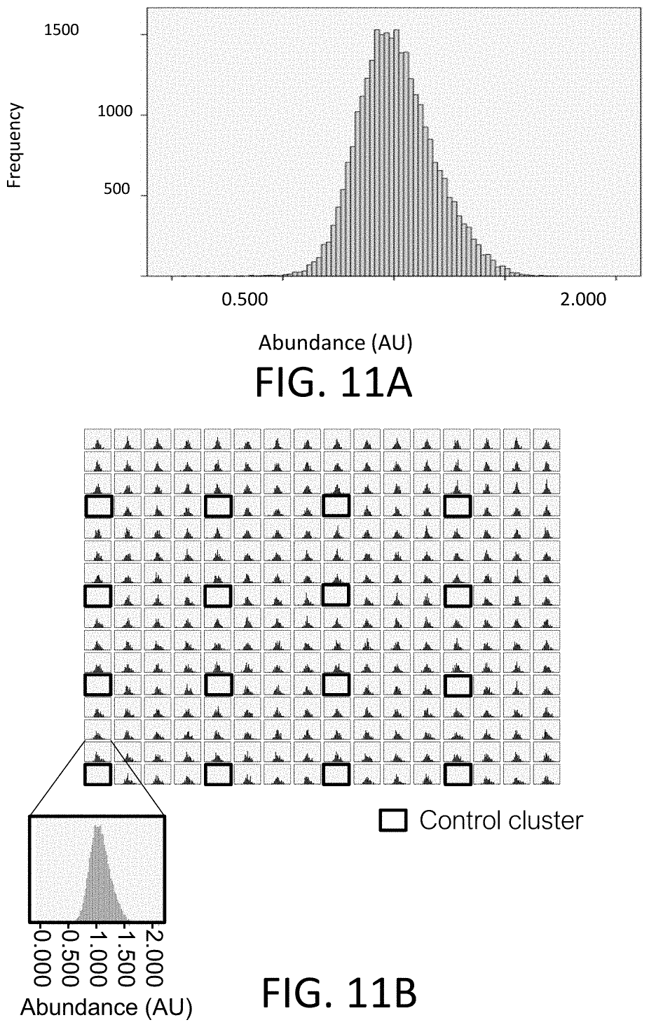

Described herein are methods of synthesis of polynucleotides from a surface, e.g., a plate (FIG. 10). In some instances, the polynucleotides are synthesized on a cluster of loci for polynucleotide extension, released and then subsequently subjected to an amplification reaction, e.g., PCR. An exemplary workflow of synthesis of polynucleotides from a cluster is depicted in FIG. 10. A silicon plate 1001 includes multiple clusters 1003. Within each cluster are multiple loci 1021. Polynucleotides are synthesized 1007 de novo on a plate 1001 from the cluster 1003. Polynucleotides are cleaved 1011 and removed 1013 from the plate to form a population of released polynucleotides 1015. The population of released polynucleotides 1015 is then amplified 1017 to form a library of amplified polynucleotides 1019.

Provided herein are methods where amplification of polynucleotides synthesized on a cluster provide for enhanced control over polynucleotide representation compared to amplification of polynucleotides across an entire surface of a structure without such a clustered arrangement. In some instances, amplification of polynucleotides synthesized from a surface having a clustered arrangement of loci for polynucleotides extension provides for overcoming the negative effects on representation due to repeated synthesis of large polynucleotide populations. Exemplary negative effects on representation due to repeated synthesis of large polynucleotide populations include, without limitation, amplification bias resulting from high/low GC content, repeating sequences, trailing adenines, secondary structure, affinity for target sequence binding, or modified nucleotides in the polynucleotide sequence.

Cluster amplification as opposed to amplification of polynucleotides across an entire plate without a clustered arrangement can result in a tighter distribution around the mean. For example, if 100,000 reads are randomly sampled, an average of 8 reads per sequence would yield a library with a distribution of about 1.5× from the mean. In some cases, single cluster amplification results in at most about 1.5×, 1.6×, 1.7×, 1.8×, 1.9×, or 2.0× from the mean. In some cases, single cluster amplification results in at least about 1.0×, 1.2×, 1.3×, 1.5× 1.6×, 1.7×, 1.8×, 1.9×, or 2.0× from the mean.

Cluster amplification methods described herein when compared to amplification across a plate can result in a polynucleotide library that requires less sequencing for equivalent sequence representation. In some instances at least 10%, at least 20%, at least 30%, at least 40%, at least 50%, at least 60%, at least 70%, at least 80%, at least 90%, or at least 95% less sequencing is required. In some instances up to 10%, up to 20%, up to 30%, up to 40%, up to 50%, up to 60%, up to 70%, up to 80%, up to 90%, or up to 95% less sequencing is required. Sometimes 30% less sequencing is required following cluster amplification compared to amplification across a plate. Sequencing of polynucleotides in some instances is verified by high-throughput sequencing such as by next generation sequencing. Sequencing of the sequencing library can be performed with any appropriate sequencing technology, including but not limited to single-molecule real-time (SMRT) sequencing, polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. The number of times a single nucleotide or polynucleotide is identified or “read” is defined as the sequencing depth or read depth. In some cases, the read depth is referred to as a fold coverage, for example, 55 fold (or 55×) coverage, optionally describing a percentage of bases.

In some instances, amplification from a clustered arrangement compared to amplification across a plate results in less dropouts, or sequences which are not detected after sequencing of amplification product. Dropouts can be of AT and/or GC. In some instances, a number of dropouts are at most about 1%, 2%, 3%, 4%, or 5% of a polynucleotide population. In some cases, the number of dropouts is zero.

A cluster as described herein comprises a collection of discrete, non-overlapping loci for polynucleotide synthesis. A cluster can comprise about 50-1000, 75-900, 100-800, 125-700, 150-600, 200-500, or 300-400 loci. In some instances, each cluster includes 121 loci. In some instances, each cluster includes about 50-500, 50-200, 100-150 loci. In some instances, each cluster includes at least about 50, 100, 150, 200, 500, 1000 or more loci. In some instances, a single plate includes 100, 500, 10000, 20000, 30000, 50000, 100000, 500000, 700000, 1000000 or more loci. A locus can be a spot, well, microwell, channel, or post. In some instances, each cluster has at least 1×, 2×, 3×, 4×, 5×, 6×, 7×, 8×, 9×, 10×, or more redundancy of separate features supporting extension of polynucleotides having identical sequence.

Generation of Polynucleotide Libraries with Controlled Stoichiometry of Sequence Content

In some instances, the polynucleotide library is synthesized with a specified distribution of desired polynucleotide sequences. In some instances, adjusting polynucleotide libraries for enrichment of specific desired sequences results in improved downstream application outcomes.

One or more specific sequences can be selected based on their evaluation in a downstream application. In some instances, the evaluation is binding affinity to target sequences for amplification, enrichment, or detection, stability, melting temperature, biological activity, ability to assemble into larger fragments, or other property of polynucleotides. In some instances, the evaluation is empirical or predicted from prior experiments and/or computer algorithms. An exemplary application includes increasing sequences in a probe library which correspond to areas of a genomic target having less than average read depth.

Selected sequences in a polynucleotide library can be at least 10%, 20%, 30%, 40%, 50%, 60%, 70%, 80%, 90%, 95%, or more than 95% of the sequences. In some instances, selected sequences in a polynucleotide library are at most 10%, 20%, 30%, 40%, 50%, 60%, 70%, 80%, 90%, 95%, or at most 100% of the sequences. In some cases, selected sequences are in a range of about 5-95%, 10-90%, 30-80%, 40-75%, or 50-70% of the sequences.

Polynucleotide libraries can be adjusted for the frequency of each selected sequence. In some instances, polynucleotide libraries favor a higher number of selected sequences. For example, a library is designed where increased polynucleotide frequency of selected sequences is in a range of about 40% to about 90%. In some instances, polynucleotide libraries contain a low number of selected sequences. For example, a library is designed where increased polynucleotide frequency of the selected sequences is in a range of about 10% to about 60%. A library can be designed to favor a higher and lower frequency of selected sequences. In some instances, a library favors uniform sequence representation. For example, polynucleotide frequency is uniform with regard to selected sequence frequency, in a range of about 10% to about 90%. In some instances, a library comprises polynucleotides with a selected sequence frequency of about 10% to about 95% of the sequences.

Generation of polynucleotide libraries with a specified selected sequence frequency in some cases occurs by combining at least 2 polynucleotide libraries with different selected sequence frequency content. In some instances, at least 2, 3, 4, 5, 6, 7, 10, or more than 10 polynucleotide libraries are combined to generate a population of polynucleotides with a specified selected sequence frequency. In some cases, no more than 2, 3, 4, 5, 6, 7, or 10 polynucleotide libraries are combined to generate a population of non-identical polynucleotides with a specified selected sequence frequency.

In some instances, selected sequence frequency is adjusted by synthesizing fewer or more polynucleotides per cluster. For example, at least 25, 50, 100, 200, 300, 400, 500, 600, 700, 800, 900, 1000, or more than 1000 non-identical polynucleotides are synthesized on a single cluster. In some cases, no more than about 50, 100, 200, 300, 400, 500, 600, 700, 800, 900, 1000 non-identical polynucleotides are synthesized on a single cluster. In some instances, 50 to 500 non-identical polynucleotides are synthesized on a single cluster. In some instances, 100 to 200 non-identical polynucleotides are synthesized on a single cluster. In some instances, about 100, about 120, about 125, about 130, about 150, about 175, or about 200 non-identical polynucleotides are synthesized on a single cluster.

In some cases, selected sequence frequency is adjusted by synthesizing non-identical polynucleotides of varying length. For example, the length of each of the non-identical polynucleotides synthesized may be at least or about at least 10, 15, 20, 25, 30, 35, 40, 45, 50, 100, 150, 200, 300, 400, 500, 2000 nucleotides, or more. The length of the non-identical polynucleotides synthesized may be at most or about at most 2000, 500, 400, 300, 200, 150, 100, 50, 45, 35, 30, 25, 20, 19, 18, 17, 16, 15, 14, 13, 12, 11, 10 nucleotides, or less. The length of each of the non-identical polynucleotides synthesized may fall from 10-2000, 10-500, 9-400, 11-300, 12-200, 13-150, 14-100, 15-50, 16-45, 17-40, 18-35, and 19-25.

Polynucleotide Probe Structures

Libraries of polynucleotide probes can be used to enrich particular target sequences in a larger population of sample polynucleotides. In some instances, polynucleotide probes each comprise a target binding sequence complementary to one or more target sequences, one or more non-target binding sequences, and one or more primer binding sites, such as universal primer binding sites. Target binding sequences that are complementary or at least partially complementary in some instances bind (hybridize) to target sequences. Primer binding sites, such as universal primer binding sites facilitate simultaneous amplification of all members of the probe library, or a subpopulation of members. In some instances, the probes or adapters further comprise a barcode or index sequence. Barcodes are nucleic acid sequences that allow some feature of a polynucleotide with which the barcode is associated to be identified. After sequencing, the barcode region provides an indicator for identifying a characteristic associated with the coding region or sample source. Barcodes can be designed at suitable lengths to allow sufficient degree of identification, e.g., at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or more bases in length. Multiple barcodes, such as about 2, 3, 4, 5, 6, 7, 8, 9, 10, or more barcodes, may be used on the same molecule, optionally separated by non-barcode sequences. In some instances, each barcode in a plurality of barcodes differ from every other barcode in the plurality at least three base positions, such as at least about 3, 4, 5, 6, 7, 8, 9, 10, or more positions. Use of barcodes allows for the pooling and simultaneous processing of multiple libraries for downstream applications, such as sequencing (multiplex). In some instances, at least 4, 8, 16, 32, 48, 64, 128, 512, 1024, 2000, 5000, or more than 5000 barcoded libraries are used. In some instances, the polynucleotides are ligated to one or more molecular (or affinity) tags such as a small molecule, peptide, antigen, metal, or protein to form a probe for subsequent capture of the target sequences of interest. In some instances, only a portion of the polynucleotides are ligated to a molecular tag. In some instances, two probes that possess complementary target binding sequences which are capable of hybridization form a double stranded probe pair. Polynucleotide probes or adapters may comprise unique molecular identifiers (UMI). UMIs allow for internal measurement of initial sample concentrations or stoichiometry prior to downstream sample processing (e.g., PCR or enrichment steps) which can introduce bias. In some instances, UMIs comprise one or more barcode sequences.

Probes described here may be complementary to target sequences which are sequences in a genome. Probes described here may be complementary to target sequences which are exome sequences in a genome. Probes described here may be complementary to target sequences which are intron sequences in a genome. In some instances, probes comprise a target binding sequence complementary to a target sequence (of the sample nucleic acid), and at least one non-target binding sequence that is not complementary to the target. In some instances, the target binding sequence of the probe is about 120 nucleotides in length, or at least 10, 15, 20, 25, 50, 75, 100, 110, 120, 125, 140, 150, 160, 175, 200, 300, 400, 500, or more than 500 nucleotides in length. The target binding sequence is in some instances no more than 10, 15, 20, 25, 50, 75, 100, 125, 150, 175, 200, or no more than 500 nucleotides in length. The target binding sequence of the probe is in some instances about 120 nucleotides in length, or about 10, 15, 20, 25, 40, 50, 60, 70, 80, 85, 87, 90, 95, 97, 100, 105, 110, 115, 117, 118, 119, 120, 121, 122, 123, 124, 125, 126, 127, 128, 129, 130, 135, 140, 145, 150, 155, 157, 158, 159, 160, 161, 162, 163, 164, 165, 166, 167, 168, 169, 170, 175, 180, 190, 200, 210, 220, 230, 240, 250, 300, 400, or about 500 nucleotides in length. The target binding sequence is in some instances about 20 to about 400 nucleotides in length, or about 30 to about 175, about 40 to about 160, about 50 to about 150, about 75 to about 130, about 90 to about 120, or about 100 to about 140 nucleotides in length. The non-target binding sequence(s) of the probe is in some instances at least about 20 nucleotides in length, or at least about 1, 5, 10, 15, 17, 20, 23, 25, 50, 75, 100, 110, 120, 125, 140, 150, 160, 175, or more than about 175 nucleotides in length. The non-target binding sequence often is no more than about 5, 10, 15, 20, 25, 50, 75, 100, 125, 150, 175, or no more than about 200 nucleotides in length. The non-target binding sequence of the probe often is about 20 nucleotides in length, or about 1, 2, 3, 4, 5, 6, 7, 8, 9, 10, 11, 12, 13, 14, 15, 16, 17, 18, 19, 20, 21, 22, 23, 25, 40, 50, 60, 70, 80, 90, 100, 110, 120, 130, 140, 150, or about 200 nucleotides in length. The non-target binding sequence in some instances is about 1 to about 250 nucleotides in length, or about 20 to about 200, about 10 to about 100, about 10 to about 50, about 30 to about 100, about 5 to about 40, or about 15 to about 35 nucleotides in length. The non-target binding sequence often comprises sequences that are not complementary to the target sequence, and/or comprise sequences that are not used to bind primers. In some instances, the non-target binding sequence comprises a repeat of a single nucleotide, for example polyadenine or polythymidine. A probe often comprises none or at least one non-target binding sequence. In some instances, a probe comprises one or two non-target binding sequences. The non-target binding sequence may be adjacent to one or more target binding sequences in a probe. For example, a non-target binding sequence is located on the 5′ or 3′ end of the probe. In some instances, the non-target binding sequence is attached to a molecular tag or spacer.

In some instances, the non-target binding sequence(s) may be a primer binding site. The primer binding sites often are each at least about 20 nucleotides in length, or at least about 10, 12, 14, 16, 18, 20, 22, 24, 26, 28, 30, 32, 34, 36, 38, or at least about 40 nucleotides in length. Each primer binding site in some instances is no more than about 10, 12, 14, 16, 18, 20, 22, 24, 26, 28, 30, 32, 34, 36, 38, or no more than about 40 nucleotides in length. Each primer binding site in some instances is about 10 to about 50 nucleotides in length, or about 15 to about 40, about 20 to about 30, about 10 to about 40, about 10 to about 30, about 30 to about 50, or about 20 to about 60 nucleotides in length. In some instances the polynucleotide probes comprise at least two primer binding sites. In some instances, primer binding sites may be universal primer binding sites, wherein all probes comprise identical primer binding sequences at these sites. In some instances, a pair of polynucleotide probes targeting a particular sequence and its reverse complement (e.g., a region of genomic DNA), comprising a first target binding sequence, a second target binding sequence, a first non-target binding sequence, and a second non-target binding sequence. For example, a pair of polynucleotide probes complementary to a particular sequence (e.g., a region of genomic DNA).

In some instances, the first target binding sequence is the reverse complement of the second target binding sequence. In some instances, both target binding sequences are chemically synthesized prior to amplification. In an alternative arrangement, a pair of polynucleotide probes targeting a particular sequence and its reverse complement (e.g., a region of genomic DNA) comprise a first target binding sequence, a second target binding sequence, a first non-target binding sequence, a second non-target binding sequence, a third non-target binding sequence, and a fourth non-target binding sequence. In some instances, the first target binding sequence is the reverse complement of the second target binding sequence. In some instances, one or more non-target binding sequences comprise polyadenine or polythymidine.

In some instances, both probes in the pair are labeled with at least one molecular tag. In some instances, PCR is used to introduce molecular tags (via primers comprising the molecular tag) onto the probes during amplification. In some instances, the molecular tag comprises one or more biotin, folate, a polyhistidine, a FLAG tag, glutathione, or other molecular tag consistent with the specification. In some instances probes are labeled at the 5′ terminus. In some instances, the probes are labeled at the 3′ terminus. In some instances, both the 5′ and 3′ termini are labeled with a molecular tag. In some instances, the 5′ terminus of a first probe in a pair is labeled with at least one molecular tag, and the 3′ terminus of a second probe in the pair is labeled with at least one molecular tag. In some instances, a spacer is present between one or more molecular tags and the nucleic acids of the probe. In some instances, the spacer may comprise an alkyl, polyol, or polyamino chain, a peptide, or a polynucleotide. The solid support used to capture probe-target nucleic acid complexes in some instances, is a bead or a surface. The solid support in some instances comprises glass, plastic, or other material capable of comprising a capture moiety that will bind the molecular tag. In some instances, a bead is a magnetic bead. For example, probes labeled with biotin are captured with a magnetic bead comprising streptavidin. The probes are contacted with a library of nucleic acids to allow binding of the probes to target sequences. In some instances, blocking polynucleotides are added to prevent binding of the probes to one or more adapter sequences attached to the target nucleic acids. In some instances, blocking polynucleotides comprise one or more nucleic acid analogues. In some instances, blocking polynucleotides have a uracil substituted for thymine at one or more positions.

Probes described herein may comprise complementary target binding sequences which bind to one or more target nucleic acid sequences. In some instances, the target sequences are any DNA or RNA nucleic acid sequence. In some instances, target sequences may be longer than the probe insert. In some instance, target sequences may be shorter than the probe insert. In some instance, target sequences may be the same length as the probe insert. For example, the length of the target sequence may be at least or about at least 2, 10, 15, 20, 25, 30, 35, 40, 45, 50, 100, 150, 200, 300, 400, 500, 1000, 2000, 5,000, 12,000, 20,000 nucleotides, or more. The length of the target sequence may be at most or about at most 20,000, 12,000, 5,000, 2,000, 1,000, 500, 400, 300, 200, 150, 100, 50, 45, 35, 30, 25, 20, 19, 18, 17, 16, 15, 14, 13, 12, 11, 10, 2 nucleotides, or less. The length of the target sequence may fall from 2-20,000, 3-12,000, 5-5, 5000, 10-2,000, 10-1,000, 10-500, 9-400, 11-300, 12-200, 13-150, 14-100, 15-50, 16-45, 17-40, 18-35, and 19-25. The probe sequences may target sequences associated with specific genes, diseases, regulatory pathways, or other biological functions consistent with the specification.

In some instances, a single probe insert is complementary to one or more target sequences in a larger polynucleic acid (e.g., sample nucleic acid). An exemplary target sequence is an exon. In some instances, one or more probes target a single target sequence. In some instances, a single probe may target more than one target sequence. In some instances, the target binding sequence of the probe targets both a target sequence and an adjacent sequence. In some instances, a first probe targets a first region and a second region of a target sequence, and a second probe targets the second region and a third region of the target sequence. In some instances, a plurality of probes targets a single target sequence, wherein the target binding sequences of the plurality of probes contain one or more sequences which overlap with regard to complementarity to a region of the target sequence. In some instances, probe inserts do not overlap with regard to complementarity to a region of the target sequence. In some instances, at least at least 2, 10, 15, 20, 25, 30, 35, 40, 45, 50, 100, 150, 200, 300, 400, 500, 1000, 2000, 5,000, 12,000, 20,000, or more than 20,000 probes target a single target sequence. In some instances no more than 4 probes directed to a single target sequence overlap, or no more than 3, 2, 1, or no probes targeting a single target sequence overlap. In some instances, one or more probes do not target all bases in a target sequence, leaving one or more gaps. In some instances, the gaps are near the middle of the target sequence. In some instances, the gaps are at the 5′ or 3′ ends of the target sequence. In some instances, the gaps are 6 nucleotides in length. In some instances, the gaps are no more than 1, 2, 3, 4, 5, 6, 7, 8, 9, 10, 20, 30, 40, or no more than 50 nucleotides in length. In some instances, the gaps are at least 1, 2, 3, 4, 5, 6, 7, 8, 9, 10, 20, 30, 40, or at least 50 nucleotides in length. In some instances, the gap length falls within 1-50, 1-40, 1-30, 1-20, 1-10, 2-30, 2-20, 2-10, 3-50, 3-25, 3-10, or 3-8 nucleotides in length. In some instances, a set of probes targeting a sequence do not comprise overlapping regions amongst probes in the set when hybridized to complementary sequence. In some instances, a set of probes targeting a sequence do not have any gaps amongst probes in the set when hybridized to complementary sequence. Probes may be designed to maximize uniform binding to target sequences. In some instances, probes are designed to minimize target binding sequences of high or low GC content, secondary structure, repetitive/palindromic sequences, or other sequence feature that may interfere with probe binding to a target. In some instances, a single probe may target a plurality of target sequences.

A probe library described herein may comprise at least 10, 20, 50, 100, 200, 500, 1,000, 2,000, 5,000, 10,000, 20,000, 50,000, 100,000, 200,000, 500,000, 1,000,000 or more than 1,000,000 probes. A probe library may have no more than 10, 20, 50, 100, 200, 500, 1,000, 2,000, 5,000, 10,000, 20,000, 50,000, 100,000, 200,000, 500,000, or no more than 1,000,000 probes. A probe library may comprise 10 to 500, 20 to 1000, 50 to 2000, 100 to 5000, 500 to 10,000, 1,000 to 5,000, 10,000 to 50,000, 100,000 to 500,000, or 50,000 to 1,000,000 probes. A probe library may comprise about 370,000; 400,000; 500,000 or more different probes.

Next Generation Sequencing Applications

Downstream applications of polynucleotide libraries may include next generation sequencing. For example, enrichment of target sequences with a controlled stoichiometry polynucleotide probe library results in more efficient sequencing. The performance of a polynucleotide library for capturing or hybridizing to targets may be defined by a number of different metrics describing efficiency, accuracy, and precision. For example, Picard metrics comprise variables such as HS library size (the number of unique molecules in the library that correspond to target regions, calculated from read pairs), mean target coverage (the percentage of bases reaching a specific coverage level), depth of coverage (number of reads including a given nucleotide) fold enrichment (sequence reads mapping uniquely to the target/reads mapping to the total sample, multiplied by the total sample length/target length), percent off-bait bases (percent of bases not corresponding to bases of the probes/baits), percent off-target (percent of bases not corresponding to bases of interest), usable bases on target, AT or GC dropout rate, fold 80 base penalty (fold over-coverage needed to raise 80 percent of non-zero targets to the mean coverage level), percent zero coverage targets, PF reads (the number of reads passing a quality filter), percent selected bases (the sum of on-bait bases and near-bait bases divided by the total aligned bases), percent duplication, or other variable consistent with the specification.

Read depth (sequencing depth, or sampling) represents the total number of times a sequenced nucleic acid fragment (a “read”) is obtained for a sequence. Theoretical read depth is defined as the expected number of times the same nucleotide is read, assuming reads are perfectly distributed throughout an idealized genome. Read depth is expressed as function of % coverage (or coverage breadth). For example, 10 million reads of a 1 million base genome, perfectly distributed, theoretically results in 10× read depth of 100% of the sequences. In practice, a greater number of reads (higher theoretical read depth, or oversampling) may be needed to obtain the desired read depth for a percentage of the target sequences. Enrichment of target sequences with a controlled stoichiometry probe library increases the efficiency of downstream sequencing, as fewer total reads will be required to obtain an outcome with an acceptable number of reads over a desired % of target sequences. For example, in some instances 55× theoretical read depth of target sequences results in at least 30× coverage of at least 90% of the sequences. In some instances no more than 55× theoretical read depth of target sequences results in at least 30× read depth of at least 80% of the sequences. In some instances no more than 55× theoretical read depth of target sequences results in at least 30× read depth of at least 95% of the sequences. In some instances no more than 55× theoretical read depth of target sequences results in at least 10× read depth of at least 98% of the sequences. In some instances, 55× theoretical read depth of target sequences results in at least 20× read depth of at least 98% of the sequences. In some instances no more than 55× theoretical read depth of target sequences results in at least 5× read depth of at least 98% of the sequences. Increasing the concentration of probes during hybridization with targets can lead to an increase in read depth. In some instances, the concentration of probes is increased by at least 1.5×, 2.0×, 2.5×, 3×, 3.5×, 4×, 5×, or more than 5×. In some instances, increasing the probe concentration results in at least a 1000% increase, or a 20%, 30%, 40%, 50%, 60%, 70%, 80%, 90%, 100%, 200%, 300%, 500%, 750%, 1000%, or more than a 1000% increase in read depth. In some instances, increasing the probe concentration by 3× results in a 1000% increase in read depth. In some instances, sequencing is performed to achieve a theoretical read depth of at least 30×, 50×, 100×, 150×, 200×, 250×, 300×, 500×, or at least 1000×. In some instances, sequencing is performed to achieve a theoretical read depth of about 30×, 50×, 100×, 150×, 200×, 250×, 300×, 500×, or about 1000×. In some instances, sequencing is performed to achieve a theoretical read depth of no more than 30×, 50×, 100×, 150×, 200×, 250×, 300×, 500×, or no more than 1000×. In some instances, sequencing is performed to achieve an actual read depth of at least 30×, 50×, 100×, 150×, 200×, 250×, 300×, 500×, or at least 1000×. In some instances, sequencing is performed to achieve an actual read depth of no more than 30×, 50×, 100×, 150×, 200×, 250×, 300×, 500×, or no more than 1000×. In some instances, sequencing is performed to achieve an actual read depth of about 30×, 50×, 100×, 150×, 200×, 250×, 300×, 500×, or about 1000×.

On-target rate represents the percentage of sequencing reads that correspond with the desired target sequences. In some instances, a controlled stoichiometry polynucleotide probe library results in an on-target rate of at least 30%, or at least 35%, 40%, 45%, 50%, 55%, 60%, 65%, 70%, 75%, 80%, 85%, or at least 90%. Increasing the concentration of polynucleotide probes during contact with target nucleic acids leads to an increase in the on-target rate. In some instances, the concentration of probes is increased by at least 1.5×, 2.0×, 2.5×, 3×, 3.5×, 4×, 5×, or more than 5×. In some instances, increasing the probe concentration results in at least a 20% increase, or a 10%, 20%, 30%, 40%, 50%, 60%, 70%, 80%, 90%, 100%, 200%, 300%, or at least a 500% increase in on-target binding. In some instances, increasing the probe concentration by 3× results in a 20% increase in on-target rate.

Coverage uniformity is in some cases calculated as the read depth as a function of the target sequence identity. Higher coverage uniformity results in a lower number of sequencing reads needed to obtain the desired read depth. For example, a property of the target sequence may affect the read depth, for example, high or low GC or AT content, repeating sequences, trailing adenines, secondary structure, affinity for target sequence binding (for amplification, enrichment, or detection), stability, melting temperature, biological activity, ability to assemble into larger fragments, sequences containing modified nucleotides or nucleotide analogues, or any other property of polynucleotides. Enrichment of target sequences with controlled stoichiometry polynucleotide probe libraries results in higher coverage uniformity after sequencing. In some instances, 95% of the sequences have a read depth that is within 1× of the mean library read depth, or about 0.05, 0.1, 0.2, 0.5, 0.7, 1, 1.2, 1.5, 1.7 or about within 2× the mean library read depth. In some instances, 80%, 85%, 90%, 95%, 97%, or 99% of the sequences have a read depth that is within 1× of the mean.

Enrichment of Target Nucleic Acids with a Polynucleotide Probe Library

A probe library described herein may be used to enrich target polynucleotides present in a population of sample polynucleotides, for a variety of downstream applications. In one some instances, a sample is obtained from one or more sources, and the population of sample polynucleotides is isolated. Samples are obtained (by way of non-limiting example) from biological sources such as saliva, blood, tissue, skin, or completely synthetic sources. The plurality of polynucleotides obtained from the sample are fragmented, end-repaired, and adenylated to form a double stranded sample nucleic acid fragment. In some instances, end repair is accomplished by treatment with one or more enzymes, such as T4 DNA polymerase, klenow enzyme, and T4 polynucleotide kinase in an appropriate buffer. A nucleotide overhang to facilitate ligation to adapters is added, in some instances with 3′ to 5′ exo minus klenow fragment and dATP.

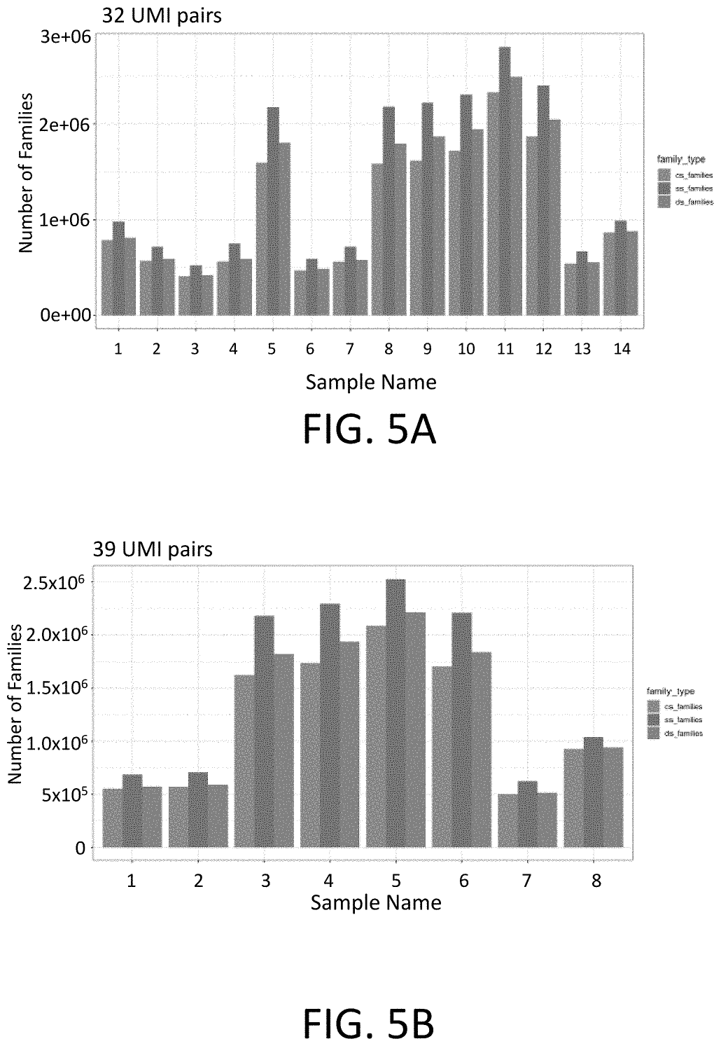

Adapters (such as universal adapters) may be ligated to both ends of the sample polynucleotide fragments with a ligase, such as T4 ligase, to produce a library of adapter-tagged polynucleotide strands, and the adapter-tagged polynucleotide library is amplified with primers, such as universal primers. In some instances, the adapters are Y-shaped adapters comprising one or more primer binding sites, one or more grafting regions, and one or more index (or barcode) regions. In some instances, the one or more index region is present on each strand of the adapter. In some instances, grafting regions are complementary to a flowcell surface, and facilitate next generation sequencing of sample libraries. In some instances, Y-shaped adapters comprise partially complementary sequences. In some instances, Y-shaped adapters comprise a single thymidine overhang which hybridizes to the overhanging adenine of the double stranded adapter-tagged polynucleotide strands. Y-shaped adapters may comprise modified nucleic acids, that are resistant to cleavage. For example, a phosphorothioate backbone is used to attach an overhanging thymidine to the 3′ end of the adapters. If universal primers are used, amplification of the library is performed to add barcoded primers to the adapters. In some instances, an enrichment workflow is depicted in FIG. 5. A library 208 of double stranded adapter-tagged polynucleotide strands 209 is contacted with polynucleotide probes 217, to form hybrid pairs 218. Such pairs are separated 212 from unhybridized fragments, and isolated from probes to produce an enriched library 213. The enriched library may then be sequenced 214.

The library of double stranded sample nucleic acid fragments is then denatured in the presence of adapter blockers. Adapter blockers minimize off-target hybridization of probes to the adapter sequences (instead of target sequences) present on the adapter-tagged polynucleotide strands, and/or prevent intermolecular hybridization of adapters (i.e., “daisy chaining”). Denaturation is carried out in some instances at 96° C., or at about 85, 87, 90, 92, 95, 97, 98 or about 99° C. A polynucleotide targeting library (probe library) is denatured in a hybridization solution, in some instances at 96° C., at about 85, 87, 90, 92, 95, 97, 98 or 99° C. The denatured adapter-tagged polynucleotide library and the hybridization solution are incubated for a suitable amount of time and at a suitable temperature to allow the probes to hybridize with their complementary target sequences. In some instances, a suitable hybridization temperature is about 45 to 80° C., or at least 45, 50, 55, 60, 65, 70, 75, 80, 85, or 90° C. In some instances, the hybridization temperature is 70° C. In some instances, a suitable hybridization time is 16 hours, or at least 4, 6, 8, 10, 12, 14, 16, 18, 20, 22, or more than 22 hours, or about 12 to 20 hours. Binding buffer is then added to the hybridized adapter-tagged-polynucleotide probes, and a solid support comprising a capture moiety is used to selectively bind the hybridized adapter-tagged polynucleotide-probes. The solid support is washed with buffer to remove unbound polynucleotides before an elution buffer is added to release the enriched, tagged polynucleotide fragments from the solid support. In some instances, the solid support is washed 2 times, or 1, 2, 3, 4, 5, or 6 times. The enriched library of adapter-tagged polynucleotide fragments is amplified and the enriched library is sequenced.

A plurality of nucleic acids (i.e. genomic sequence) may obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. The adapter-tagged polynucleotide library is then denatured at high temperature, preferably 96° C., in the presence of adapter blockers. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched library of adapter-tagged polynucleotide fragments is amplified and then the library is sequenced. Alternative variables such as incubation times, temperatures, reaction volumes/concentrations, number of washes, or other variables consistent with the specification are also employed in the method.

In any of the instances, the detection or quantification analysis of the oligonucleotides can be accomplished by sequencing. The subunits or entire synthesized oligonucleotides can be detected via full sequencing of all oligonucleotides by any suitable methods known in the art, e.g., Illumina sequencing by synthesis, PacBio nanopore sequencing, or BM/MGI nanoball sequencing, including the sequencing methods described herein.

Sequencing can be accomplished through classic Sanger sequencing methods which are well known in the art. Sequencing can also be accomplished using high-throughput systems some of which allow detection of a sequenced nucleotide immediately after or upon its incorporation into a growing strand, i.e., detection of sequence in red time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read.

In some instances, high-throughput sequencing involves the use of technology available by Illumina's Genome Analyzer IIX, MiSeq personal sequencer, or HiSeq systems, such as those using HiSeq 2500, HiSeq 1500, HiSeq 2000, HiSeq 1000, iSeq 100, Mini Seq, MiSeq, NextSeq 550, NextSeq 2000, NextSeq 550, or NovaSeq 6000. These machines use reversible terminator-based sequencing by synthesis chemistry. These machines can generate 6000 Gb or more reads in 13-44 hours. Smaller systems may be utilized for runs within 3, 2, 1 days or less time. Short synthesis cycles may be used to minimize the time it takes to obtain sequencing results.

In some instances, high-throughput sequencing involves the use of technology available by ABI Solid System. This genetic analysis platform that enables massively parallel sequencing of clonally-amplified DNA fragments linked to beads. The sequencing methodology is based on sequential ligation with dye-labeled oligonucleotides.

The next generation sequencing can comprise ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some cases, an IONPROTON™ Sequencer is used to sequence nucleic acid. In some cases, an IONPGM™ Sequencer is used. The Ion Torrent Personal Genome Machine (PGM) can do 10 million reads in two hours.

In some instances, high-throughput sequencing involves the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. Finally, SMSS is powerful because, like the MW technology, it does not require a pre amplification step prior to hybridization. In fact, SMSS does not require any amplification.

In some instances, high-throughput sequencing involves the use of technology available by 454 Lifesciences, Inc. (Branford, Conn.) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles, M., et al. “Genome sequencing in microfabricated high-density picolitre reactors”, Nature, doi: 10.1038/nature03959.

In some instances, high-throughput sequencing is performed using Clonal Single Molecule Array (Solexa, Inc.) or sequencing-by-synthesis (SBS) utilizing reversible terminator chemistry. Constans, A., The Scientist 2003, 17(13):36. High-throughput sequencing of oligonucleotides can be achieved using any suitable sequencing method known in the art, such as those commercialized by Pacific Biosciences, Complete Genomics, Genia Technologies, Halcyon Molecular, Oxford Nanopore Technologies and the like. Overall such systems involve sequencing a target oligonucleotide molecule having a plurality of bases by the temporal addition of bases via a polymerization reaction that is measured on a molecule of oligonucleotide, i e., the activity of a nucleic acid polymerizing enzyme on the template oligonucleotide molecule to be sequenced is followed in real time. Sequence can then be deduced by identifying which base is being incorporated into the growing complementary strand of the target oligonucleotide by the catalytic activity of the nucleic acid polymerizing enzyme at each step in the sequence of base additions. A polymerase on the target oligonucleotide molecule complex is provided in a position suitable to move along the target oligonucleotide molecule and extend the oligonucleotide primer at an active site. A plurality of labeled types of nucleotide analogs are provided proximate to the active site, with each distinguishably type of nucleotide analog being complementary to a different nucleotide in the target oligonucleotide sequence. The growing oligonucleotide strand is extended by using the polymerase to add a nucleotide analog to the oligonucleotide strand at the active site, where the nucleotide analog being added is complementary to the nucleotide of the target oligonucleotide at the active site. The nucleotide analog added to the oligonucleotide primer as a result of the polymerizing step is identified. The steps of providing labeled nucleotide analogs, polymerizing the growing oligonucleotide strand, and identifying the added nucleotide analog are repeated so that the oligonucleotide strand is further extended and the sequence of the target oligonucleotide is determined.

The next generation sequencing technique can comprises real-time (SMRT™) technology by Pacific Biosciences. In SMRT, each of four DNA bases can be attached to one of four different fluorescent dyes. These dyes can be phospho linked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zepto liters (10″ liters) can be created. The tiny detection volume can provide 1000-fold improvement in the reduction of background noise. Detection of the corresponding fluorescence of the dye can indicate which base was incorporated. The process can be repeated.

In some cases, the next generation sequencing is nanopore sequencing {See e.g., Soni G V and Meller A. (2007) Clin Chem 53: 1996-2001). A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridION system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNₓ, or SiO₂). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some cases, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

Nanopore sequencing technology from GENIA can be used. An engineered protein pore can be embedded in a lipid bilayer membrane. “Active Control” technology can be used to enable efficient nanopore-membrane assembly and control of DNA movement through the channel. In some cases, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some cases, the nanopore sequencing technology is from IBM/Roche. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by Complete Genomics; see e.g., Drmanac et al. (2010) Science 327: 78-81). DNA can be isolated, fragmented, and size selected. For example, DNA can be fragmented (e.g., by sonication) to a mean length of about 500 bp. Adaptors (Ad1) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamethyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

A population of polynucleotides may be enriched prior to adapter ligation. In one example, a plurality of polynucleotides is obtained from a sample, fragmented, optionally end-repaired, and denatured at high temperature, preferably 90-99° C. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched polynucleotide fragments are then polyadenylated, adapters are ligated to both ends of the polynucleotide fragments to produce a library of adapter-tagged polynucleotide strands, and the adapter-tagged polynucleotide library is amplified. The adapter-tagged polynucleotide library is then sequenced.

A polynucleotide targeting library may also be used to filter undesired sequences from a plurality of polynucleotides, by hybridizing to undesired fragments. For example, a plurality of polynucleotides is obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. Alternatively, adenylation and adapter ligation steps are instead performed after enrichment of the sample polynucleotides. The adapter-tagged polynucleotide library is then denatured at high temperature, preferably 90-99° C., in the presence of adapter blockers. A polynucleotide filtering library (probe library) designed to remove undesired, non-target sequences is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 1 and 5 times to elute unbound adapter-tagged polynucleotide fragments. The enriched library of unbound adapter-tagged polynucleotide fragments is amplified and then the amplified library is sequenced.

Highly Parallel De Novo Nucleic Acid Synthesis

Described herein is a platform approach utilizing miniaturization, parallelization, and vertical integration of the end-to-end process from polynucleotide synthesis to gene assembly within Nano wells on silicon to create a revolutionary synthesis platform. Devices described herein provide, with the same footprint as a 96-well plate, a silicon synthesis platform is capable of increasing throughput by a factor of 100 to 1,000 compared to traditional synthesis methods, with production of up to approximately 1,000,000 polynucleotides in a single highly-parallelized run. In some instances, a single silicon plate described herein provides for synthesis of about 6,100 non-identical polynucleotides. In some instances, each of the non-identical polynucleotides is located within a cluster. A cluster may comprise 50 to 500 non-identical polynucleotides.

Methods described herein provide for synthesis of a library of polynucleotides each encoding for a predetermined variant of at least one predetermined reference nucleic acid sequence. In some cases, the predetermined reference sequence is nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. The synthesized specific alterations in the nucleic acid sequence can be introduced by incorporating nucleotide changes into overlapping or blunt ended polynucleotide primers. Alternatively, a population of polynucleotides may collectively encode for a long nucleic acid (e.g., a gene) and variants thereof. In this arrangement, the population of polynucleotides can be hybridized and subject to standard molecular biology techniques to form the long nucleic acid (e.g., a gene) and variants thereof. When the long nucleic acid (e.g., a gene) and variants thereof are expressed in cells, a variant protein library is generated. Similarly, provided here are methods for synthesis of variant libraries encoding for RNA sequences (e.g., miRNA, shRNA, and mRNA) or DNA sequences (e.g., enhancer, promoter, UTR, and terminator regions). Also provided here are downstream applications for variants selected out of the libraries synthesized using methods described here. Downstream applications include identification of variant nucleic acid or protein sequences with enhanced biologically relevant functions, e.g., biochemical affinity, enzymatic activity, changes in cellular activity, and for the treatment or prevention of a disease state.

Substrates

Provided herein are substrates comprising a plurality of clusters, wherein each cluster comprises a plurality of loci that support the attachment and synthesis of polynucleotides. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus refers to a discrete raised or lowered site on a surface e.g., a well, micro well, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some instances, a surface of a device is inclusive of one or a plurality of surfaces of a substrate.

Provided herein are structures that may comprise a surface that supports the synthesis of a plurality of polynucleotides having different predetermined sequences at addressable locations on a common support. In some instances, a device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some instances, the device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence.

Provided herein are methods and devices for manufacture and growth of polynucleotides about 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, or 2000 bases in length. In some instances, the length of the polynucleotide formed is about 5, 10, 20, 30, 40, 50, 60, 70, 80, 90, 100, 125, 150, 175, 200, or 225 bases in length. A polynucleotide may be at least 5, 10, 20, 30, 40, 50, 60, 70, 80, 90, or 100 bases in length. A polynucleotide may be from 10 to 225 bases in length, from 12 to 100 bases in length, from 20 to 150 bases in length, from 20 to 130 bases in length, or from 30 to 100 bases in length.

In some instances, polynucleotides are synthesized on distinct loci of a substrate, wherein each locus supports the synthesis of a population of polynucleotides. In some instances, each locus supports the synthesis of a population of polynucleotides having a different sequence than a population of polynucleotides grown on another locus. In some instances, the loci of a device are located within a plurality of clusters. In some instances, a device comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a device comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a device comprises about 10,000 distinct loci. The amount of loci within a single cluster is varied in different instances. In some instances, each cluster includes 1, 2, 3, 4, 5, 6, 7, 8, 9, 10, 20, 30, 40, 50, 60, 70, 80, 90, 100, 120, 130, 150, 200, 300, 400, 500, 1000 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci.

The number of distinct polynucleotides synthesized on a device may be dependent on the number of distinct loci available in the substrate. In some instances, the density of loci within a cluster of a device is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm² or more. In some instances, a device comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some instances, the distance from the centers of two adjacent loci within a cluster is from about 10 um to about 500 um, from about 10 um to about 200 um, or from about 10 um to about 100 um. In some instances, the distance from two centers of adjacent loci is greater than about 10 um, 20 um, 30 um, 40 um, 50 um, 60 um, 70 um, 80 um, 90 um or 100 um. In some instances, the distance from the centers of two adjacent loci is less than about 200 um, 150 um, 100 um, 80 um, 70 um, 60 um, 50 um, 40 um, 30 um, 20 um or 10 um. In some instances, each locus has a width of about 0.5 μm, 1 um, 2 um, 3 um, 4 um, 5 um, 6 um, 7 um, 8 um, 9 um, 10 um, 20 um, 30 um, 40 um, 50 um, 60 um, 70 um, 80 um, 90 um or 100 um. In some instances, each locus is has a width of about 0.5 um to 100 um, about 0.5 um to 50 um, about 10 um to 75 um, or about 0.5 um to 50 um.

In some instances, the density of clusters within a device is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a device comprises from about 1 cluster per 10 mm² to about 10 clusters per 1 mm². In some instances, the distance from the centers of two adjacent clusters is less than about 50 um, 100 um, 200 um, 500 um, 1000 um, or 2000 um or 5000 um. In some instances, the distance from the centers of two adjacent clusters is from about 50 um and about 100 um, from about 50 um and about 200 um, from about 50 um and about 300 um, from about 50 um and about 500 um, and from about 100 μm to about 2000 um. In some instances, the distance from the centers of two adjacent clusters is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, each cluster has a diameter or width along one dimension of about 0.5 to 2 mm, about 0.5 to 1 mm, or about 1 to 2 mm. In some instances, each cluster has a diameter or width along one dimension of about 0.5, 0.6, 0.7, 0.8, 0.9, 1, 1.1, 1.2, 1.3, 1.4, 1.5, 1.6, 1.7, 1.8, 1.9 or 2 mm. In some instances, each cluster has an interior diameter or width along one dimension of about 0.5, 0.6, 0.7, 0.8, 0.9, 1, 1.1, 1.15, 1.2, 1.3, 1.4, 1.5, 1.6, 1.7, 1.8, 1.9 or 2 mm.

A device may be about the size of a standard 96 well plate, for example from about 100 and 200 mm by from about 50 and 150 mm. In some instances, a device has a diameter less than or equal to about 1000 mm, 500 mm, 450 mm, 400 mm, 300 mm, 250 nm, 200 mm, 150 mm, 100 mm or 50 mm. In some instances, the diameter of a device is from about 25 mm and 1000 mm, from about 25 mm and about 800 mm, from about 25 mm and about 600 mm, from about 25 mm and about 500 mm, from about 25 mm and about 400 mm, from about 25 mm and about 300 mm, or from about 25 mm and about 200. Non-limiting examples of device size include about 300 mm, 200 mm, 150 mm, 130 mm, 100 mm, 76 mm, 51 mm and 25 mm. In some instances, a device has a planar surface area of at least about 100 mm²; 200 mm²; 500 mm²; 1,000 mm²; 2,000 mm²; 5,000 mm²; 10,000 mm²; 12,000 mm²; 15,000 mm²; 20,000 mm²; 30,000 mm²; 40,000 mm²; 50,000 mm² or more. In some instances, the thickness of a device is from about 50 mm and about 2000 mm, from about 50 mm and about 1000 mm, from about 100 mm and about 1000 mm, from about 200 mm and about 1000 mm, or from about 250 mm and about 1000 mm. Non-limiting examples of device thickness include 275 mm, 375 mm, 525 mm, 625 mm, 675 mm, 725 mm, 775 mm and 925 mm. In some instances, the thickness of a device varies with diameter and depends on the composition of the substrate. For example, a device comprising materials other than silicon has a different thickness than a silicon device of the same diameter. Device thickness may be determined by the mechanical strength of the material used and the device must be thick enough to support its own weight without cracking during handling. In some instances, a structure comprises a plurality of devices described herein.

Surface Materials

Provided herein is a device comprising a surface, wherein the surface is modified to support polynucleotide synthesis at predetermined locations and with a resulting low error rate, a low dropout rate, a high yield, and a high oligo representation. In some instances, surfaces of a device for polynucleotide synthesis provided herein are fabricated from a variety of materials capable of modification to support a de novo polynucleotide synthesis reaction. In some cases, the devices are sufficiently conductive, e.g., are able to form uniform electric fields across all or a portion of the device. A device described herein may comprise a flexible material. Exemplary flexible materials include, without limitation, modified nylon, unmodified nylon, nitrocellulose, and polypropylene. A device described herein may comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, gold, platinum). Device disclosed herein may be fabricated from a material comprising silicon, polystyrene, agarose, dextran, cellulosic polymers, polyacrylamides, polydimethylsiloxane (PDMS), glass, or any combination thereof. In some cases, a device disclosed herein is manufactured with a combination of materials listed herein or any other suitable material known in the art.

A listing of tensile strengths for exemplary materials described herein is provides as follows: nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for polynucleotide synthesis that is in the form of a flexible material capable of being stored in a continuous loop or reel, such as a tape or flexible sheet.

Young's modulus measures the resistance of a material to elastic (recoverable) deformation under load. A listing of Young's modulus for stiffness of exemplary materials described herein is provides as follows: nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load.

In some cases, a device disclosed herein comprises a silicon dioxide base and a surface layer of silicon oxide. Alternatively, the device may have a base of silicon oxide. Surface of the device provided here may be textured, resulting in an increase overall surface area for polynucleotide synthesis. Device disclosed herein may comprise at least 5%, 10%, 25%, 50%, 80%, 90%, 95%, or 99% silicon. A device disclosed herein may be fabricated from a silicon on insulator (SOI) wafer.

Surface Architecture

Provided herein are devices comprising raised and/or lowered features. One benefit of having such features is an increase in surface area to support polynucleotide synthesis. In some instances, a device having raised and/or lowered features is referred to as a three-dimensional substrate. In some instances, a three-dimensional device comprises one or more channels. In some instances, one or more loci comprise a channel. In some instances, the channels are accessible to reagent deposition via a deposition device such as a polynucleotide synthesizer. In some instances, reagents and/or fluids collect in a larger well in fluid communication one or more channels. For example, a device comprises a plurality of channels corresponding to a plurality of loci with a cluster, and the plurality of channels are in fluid communication with one well of the cluster. In some methods, a library of polynucleotides is synthesized in a plurality of loci of a cluster.

In some instances, the structure is configured to allow for controlled flow and mass transfer paths for polynucleotide synthesis on a surface. In some instances, the configuration of a device allows for the controlled and even distribution of mass transfer paths, chemical exposure times, and/or wash efficacy during polynucleotide synthesis. In some instances, the configuration of a device allows for increased sweep efficiency, for example by providing sufficient volume for a growing a polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are methods to synthesize an amount of DNA of 1 fM, 5 fM, 10 fM, 25 fM, 50 fM, 75 fM, 100 fM, 200 fM, 300 fM, 400 fM, 500 fM, 600 fM, 700 fM, 800 fM, 900 fM, 1 pM, 5 pM, 10 pM, 25 pM, 50 pM, 75 pM, 100 pM, 200 pM, 300 pM, 400 pM, 500 pM, 600 pM, 700 pM, 800 pM, 900 pM, or more. In some instances, a polynucleotide library may span the length of about 1%, 2%, 3%, 4%, 5%, 10%, 15%, 20%, 30%, 40%, 50%, 60%, 70%, 80%, 90%, 95%, or 100% of a gene. A gene may be varied up to about 1%, 2%, 3%, 4%, 5%, 10%, 15%, 20%, 30%, 40%, 50%, 60%, 70%, 80%, 85%, 90%, 95%, or 100%.

Non-identical polynucleotides may collectively encode a sequence for at least 1%, 2%, 3%, 4%, 5%, 10%, 15%, 20%, 30%, 40%, 50%, 60%, 70%, 80%, 85%, 90%, 95%, or 100% of a gene. In some instances, a polynucleotide may encode a sequence of 50%, 60%, 70%, 80%, 85%, 90%, 95%, or more of a gene. In some instances, a polynucleotide may encode a sequence of 80%, 85%, 90%, 95%, or more of a gene.

In some instances, segregation is achieved by physical structure. In some instances, segregation is achieved by differential functionalization of the surface generating active and passive regions for polynucleotide synthesis. Differential functionalization is also be achieved by alternating the hydrophobicity across the device surface, thereby creating water contact angle effects that cause beading or wetting of the deposited reagents. Employing larger structures can decrease splashing and cross-contamination of distinct polynucleotide synthesis locations with reagents of the neighboring spots. In some instances, a device, such as a polynucleotide synthesizer, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000; 1:3,000; 1:5,000; or 1:10,000). In some instances, a device comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a device may have the same or different width, height, and/or volume as another well of the substrate. A channel of a device may have the same or different width, height, and/or volume as another channel of the substrate. In some instances, the width of a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a well comprising a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a cluster is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a cluster is from about 1.0 and 1.3 mm. In some instances, the width of a cluster is about 1.150 mm. In some instances, the width of a well is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a well is from about 1.0 and 1.3 mm. In some instances, the width of a well is about 1.150 mm. In some instances, the width of a cluster is about 0.08 mm. In some instances, the width of a well is about 0.08 mm. The width of a cluster may refer to clusters within a two-dimensional or three-dimensional substrate.

In some instances, the height of a well is from about 20 um to about 1000 um, from about 50 um to about 1000 um, from about 100 μm to about 1000 um, from about 200 μm to about 1000 um, from about 300 μm to about 1000 um, from about 400 μm to about 1000 um, or from about 500 um to about 1000 um. In some instances, the height of a well is less than about 1000 um, less than about 900 um, less than about 800 um, less than about 700 um, or less than about 600 um.

In some instances, a device comprises a plurality of channels corresponding to a plurality of loci within a cluster, wherein the height or depth of a channel is from about 5 um to about 500 um, from about 5 um to about 400 um, from about 5 um to about 300 um, from about 5 um to about 200 um, from about 5 um to about 100 um, from about 5 um to about 50 um, or from about 10 um to about 50 um. In some instances, the height of a channel is less than 100 um, less than 80 um, less than 60 um, less than 40 um or less than 20 um.

In some instances, the diameter of a channel, locus (e.g., in a substantially planar substrate) or both channel and locus (e.g., in a three-dimensional device wherein a locus corresponds to a channel) is from about 1 um to about 1000 um, from about 1 um to about 500 um, from about 1 um to about 200 um, from about 1 um to about 100 um, from about 5 um to about 100 um, or from about 10 um to about 100 um, for example, about 90 um, 80 um, 70 um, 60 um, 50 um, 40 um, 30 um, 20 um or 10 um. In some instances, the diameter of a channel, locus, or both channel and locus is less than about 100 um, 90 um, 80 um, 70 um, 60 um, 50 um, 40 um, 30 um, 20 um or 10 um. In some instances, the distance from the center of two adjacent channels, loci, or channels and loci is from about 1 um to about 500 um, from about 1 um to about 200 um, from about 1 um to about 100 um, from about 5 um to about 200 um, from about 5 um to about 100 um, from about 5 um to about 50 um, or from about 5 um to about 30 um, for example, about 20 um.

Surface Modifications

In various instances, surface modifications are employed for the chemical and/or physical alteration of a surface by an additive or subtractive process to change one or more chemical and/or physical properties of a device surface or a selected site or region of a device surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some instances, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some instances, the adhesion promoter is a chemical with a high surface energy. In some instances, a second chemical layer is deposited on a surface of a substrate. In some instances, the second chemical layer has a low surface energy. In some instances, surface energy of a chemical layer coated on a surface supports localization of droplets on the surface. Depending on the patterning arrangement selected, the proximity of loci and/or area of fluid contact at the loci are alterable.

In some instances, a device surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a device surface is modified with one or more different layers of compounds. Such modification layers of interest include, without limitation, inorganic and organic layers such as metals, metal oxides, polymers, small organic molecules and the like. Non-limiting polymeric layers include peptides, proteins, nucleic acids or mimetics thereof (e.g., peptide nucleic acids and the like), polysaccharides, phospholipids, polyurethanes, polyesters, polycarbonates, polyureas, polyamides, polyethyleneamines, polyarylene sulfides, polysiloxanes, polyimides, polyacetates, and any other suitable compounds described herein or otherwise known in the art. In some instances, polymers are heteropolymeric. In some instances, polymers are homopolymeric. In some instances, polymers comprise functional moieties or are conjugated.

In some instances, resolved loci of a device are functionalized with one or more moieties that increase and/or decrease surface energy. In some instances, a moiety is chemically inert. In some instances, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for device functionalization may comprise: (a) providing a device having a surface that comprises silicon dioxide; and (b) silanizing the surface using, a suitable silanizing agent described herein or otherwise known in the art, for example, an organofunctional alkoxysilane molecule.

In some instances, the organofunctional alkoxysilane molecule comprises dimethylchloro-octodecyl-silane, methyldichloro-octodecyl-silane, trichloro-octodecyl-silane, trimethyl-octodecyl-silane, triethyl-octodecyl-silane, or any combination thereof. In some instances, a device surface comprises functionalized with polyethylene/polypropylene (functionalized by gamma irradiation or chromic acid oxidation, and reduction to hydroxyalkyl surface), highly crosslinked polystyrene-divinylbenzene (derivatized by chloromethylation, and aminated to benzylamine functional surface), nylon (the terminal aminohexyl groups are directly reactive), or etched with reduced polytetrafluoroethylene. Other methods and functionalizing agents are described in U.S. Pat. No. 5,474,796, which is herein incorporated by reference in its entirety.

In some instances, a device surface is functionalized by contact with a derivatizing composition that contains a mixture of silanes, under reaction conditions effective to couple the silanes to the device surface, typically via reactive hydrophilic moieties present on the device surface. Silanization generally covers a surface through self-assembly with organofunctional alkoxysilane molecules.

A variety of siloxane functionalizing reagents can further be used as currently known in the art, e.g., for lowering or increasing surface energy. The organofunctional alkoxysilanes can be classified according to their organic functions.

Provided herein are devices that may contain patterning of agents capable of coupling to a nucleoside. In some instances, a device may be coated with an active agent. In some instances, a device may be coated with a passive agent. Exemplary active agents for inclusion in coating materials described herein includes, without limitation, N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane (GOPS), 3-iodo-propyltrimethoxysilane, butyl-aldehydr-trimethoxysilane, dimeric secondary aminoalkyl siloxanes, (3-aminopropyl)-diethoxy-methylsilane, (3-aminopropyl)-dimethyl-ethoxysilane, and (3-aminopropyl)-trimethoxysilane, (3-glycidoxypropyl)-dimethyl-ethoxysilane, glycidoxy-trimethoxysilane, (3-mercaptopropyl)-trimethoxysilane, 3-4 epoxycyclohexyl-ethyltrimethoxysilane, and (3-mercaptopropyl)-methyl-dimethoxysilane, allyl trichlorochlorosilane, 7-oct-1-enyl trichlorochlorosilane, or bis (3-trimethoxysilylpropyl) amine.

Exemplary passive agents for inclusion in a coating material described herein includes, without limitation, perfluorooctyltrichlorosilane; tridecafluoro-1,1,2,2-tetrahydrooctyl)trichlorosilane; 1H, 1H, 2H, 2H-fluorooctyltriethoxysilane (FOS); trichloro(1H, 1H, 2H, 2H −perfluorooctyl)silane; tert-butyl-[5-fluoro-4-(4,4,5,5-tetramethyl-1,3,2-dioxaborolan-2-yl)indol-1-yl]-dimethyl-silane; CYTOP™; Fluorinert™; perfluoroctyltrichlorosilane (PFOTCS); perfluorooctyldimethylchlorosilane (PFODCS); perfluorodecyltriethoxysilane (PFDTES); pentafluorophenyl-dimethylpropylchloro-silane (PFPTES); perfluorooctyltriethoxysilane; perfluorooctyltrimethoxysilane; octylchlorosilane; dimethylchloro-octodecyl-silane; methyldichloro-octodecyl-silane; trichloro-octodecyl-silane; trimethyl-octodecyl-silane; triethyl-octodecyl-silane; or octadecyltrichlorosilane.

In some instances, a functionalization agent comprises a hydrocarbon silane such as octadecyltrichlorosilane. In some instances, the functionalizing agent comprises 11-acetoxyundecyltriethoxysilane, n-decyltriethoxysilane, (3-aminopropyl)trimethoxysilane, (3-aminopropyl)triethoxysilane, glycidyloxypropyl/trimethoxysilane and N-(3-triethoxysilylpropyl)-4-hydroxybutyramide.

Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once, i.e., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 minutes, 1 minute, 50 seconds, 40 seconds, 30 seconds, 20 seconds and 10 seconds.

Polynucleotide synthesis using a phosphoramidite method may comprise a subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the device is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′—OH groups after coupling from further chain elongation, preventing the formation of polynucleotides with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the device is optionally washed.

In some instances, following addition of a nucleoside phosphoramidite, and optionally after capping and one or more wash steps, the device bound growing nucleic acid is oxidized. The oxidation step comprises the phosphite triester is oxidized into a tetracoordinated phosphate triester, a protected precursor of the naturally occurring phosphate diester internucleoside linkage. In some instances, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing polynucleotide is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions of the disclosure described herein provide for controlled deblocking conditions limiting undesired depurination reactions. In some instances, the device bound polynucleotide is washed after deblocking. In some instances, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based polynucleotide synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to polynucleotide synthesis devices for the synthesis of polynucleotides. The synthesis may be in parallel. For example at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more polynucleotides can be synthesized in parallel. The total number polynucleotides that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150,22-100, 23-50, 24-45, 25-40, 30-35. Those of skill in the art appreciate that the total number of polynucleotides synthesized in parallel may fall within any range bound by any of these values, for example 25-100. The total number of polynucleotides synthesized in parallel may fall within any range defined by any of the values serving as endpoints of the range. Total molar mass of polynucleotides synthesized within the device or the molar mass of each of the polynucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the polynucleotides or average length of the polynucleotides within the device may be at least or about at least 10, 15, 20, 25, 30, 35, 40, 45, 50, 100, 150, 200, 300, 400, 500 nucleotides, or more. The length of each of the polynucleotides or average length of the polynucleotides within the device may be at most or about at most 500, 400, 300, 200, 150, 100, 50, 45, 35, 30, 25, 20, 19, 18, 17, 16, 15, 14, 13, 12, 11, 10 nucleotides, or less. The length of each of the polynucleotides or average length of the polynucleotides within the device may fall from 10-500, 9-400, 11-300, 12-200, 13-150, 14-100, 15-50, 16-45, 17-40, 18-35, 19-25. Those of skill in the art appreciate that the length of each of the polynucleotides or average length of the polynucleotides within the device may fall within any range bound by any of these values, for example 100-300. The length of each of the polynucleotides or average length of the polynucleotides within the device may fall within any range defined by any of the values serving as endpoints of the range.

Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct polynucleotides, wherein polynucleotide encoding a distinct sequence is synthesized on a resolved locus. In some instances, a library of polynucleotides are synthesized on a device with low error rates described herein in less than about three months, two months, one month, three weeks, 15, 14, 13, 12, 11, 10, 9, 8, 7, 6, 5, 4, 3, 2 days, 24 hours or less. In some instances, larger nucleic acids assembled from a polynucleotide library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some instances, methods described herein provide for generation of a library of polynucleotides comprising variant polynucleotides differing at a plurality of codon sites. In some instances, a polynucleotide may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may be not be adjacent and separated by 1, 2, 3, 4, 5, 6, 7, 8, 9, 10, or more codons.

In some instances, a polynucleotide may comprise multiple sites of variant codon sites, wherein all the variant codon sites are adjacent to one another, forming a stretch of variant codon sites. In some instances, a polynucleotide may comprise multiple sites of variant codon sites, wherein none the variant codon sites are adjacent to one another. In some instances, a polynucleotide may comprise multiple sites of variant codon sites, wherein some the variant codon sites are adjacent to one another, forming a stretch of variant codon sites, and some of the variant codon sites are not adjacent to one another.

Large Polynucleotide Libraries Having Low Error Rates

Average error rates for polynucleotides synthesized within a library using the systems and methods provided may be less than 1 in 1000, less than 1 in 1250, less than 1 in 1500, less than 1 in 2000, less than 1 in 3000 or less often. In some instances, average error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less. In some instances, average error rates for polynucleotides synthesized within a library using the systems and methods provided are less than 1/1000.

In some instances, aggregate error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less compared to the predetermined sequences. In some instances, aggregate error rates for polynucleotides synthesized within a library using the systems and methods provided are less than 1/500, 1/600, 1/700, 1/800, 1/900, or 1/1000. In some instances, aggregate error rates for polynucleotides synthesized within a library using the systems and methods provided are less than 1/1000.

In some instances, an error correction enzyme may be used for polynucleotides synthesized within a library using the systems and methods provided can use. In some instances, aggregate error rates for polynucleotides with error correction can be less than 1/500, 1/600, 1/700, 1/800, 1/900, 1/1000, 1/1100, 1/1200, 1/1300, 1/1400, 1/1500, 1/1600, 1/1700, 1/1800, 1/1900, 1/2000, 1/3000, or less compared to the predetermined sequences. In some instances, aggregate error rates with error correction for polynucleotides synthesized within a library using the systems and methods provided can be less than 1/500, 1/600, 1/700, 1/800, 1/900, or 1/1000. In some instances, aggregate error rates with error correction for polynucleotides synthesized within a library using the systems and methods provided can be less than 1/1000.

Error rate may limit the value of gene synthesis for the production of libraries of gene variants. With an error rate of 1/300, about 0.7% of the clones in a 1500 base pair gene will be correct. As most of the errors from polynucleotide synthesis result in frame-shift mutations, over 99% of the clones in such a library will not produce a full-length protein. Reducing the error rate by 75% would increase the fraction of clones that are correct by a factor of 40. The methods and compositions of the disclosure allow for fast de novo synthesis of large polynucleotide and gene libraries with error rates that are lower than commonly observed gene synthesis methods both due to the improved quality of synthesis and the applicability of error correction methods that are enabled in a massively parallel and time-efficient manner. Accordingly, libraries may be synthesized with base insertion, deletion, substitution, or total error rates that are under 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less, across the library, or across more than 80%, 85%, 90%, 93%, 95%, 96%, 97%, 98%, 99%, 99.5%, 99.8%, 99.9%, 99.95%, 99.98%, 99.99%, or more of the library. The methods and compositions of the disclosure further relate to large synthetic polynucleotide and gene libraries with low error rates associated with at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in at least a subset of the library to relate to error free sequences in comparison to a predetermined/preselected sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in an isolated volume within the library have the same sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of any polynucleotides or genes related with more than 95%, 96%, 97%, 98%, 99%, 99.5%, 99.6%, 99.7%, 99.8%, 99.9% or more similarity or identity have the same sequence. In some instances, the error rate related to a specified locus on a polynucleotide or gene is optimized. Thus, a given locus or a plurality of selected loci of one or more polynucleotides or genes as part of a large library may each have an error rate that is less than 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less. In various instances, such error optimized loci may comprise at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 50000, 75000, 100000, 500000, 1000000, 2000000, 3000000 or more loci. The error optimized loci may be distributed to at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 75000, 100000, 500000, 1000000, 2000000, 3000000 or more polynucleotides or genes.

The error rates can be achieved with or without error correction. The error rates can be achieved across the library, or across more than 80%, 85%, 90%, 93%, 95%, 96%, 97%, 98%, 99%, 99.5%, 99.8%, 99.9%, 99.95%, 99.98%, 99.99%, or more of the library.

Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

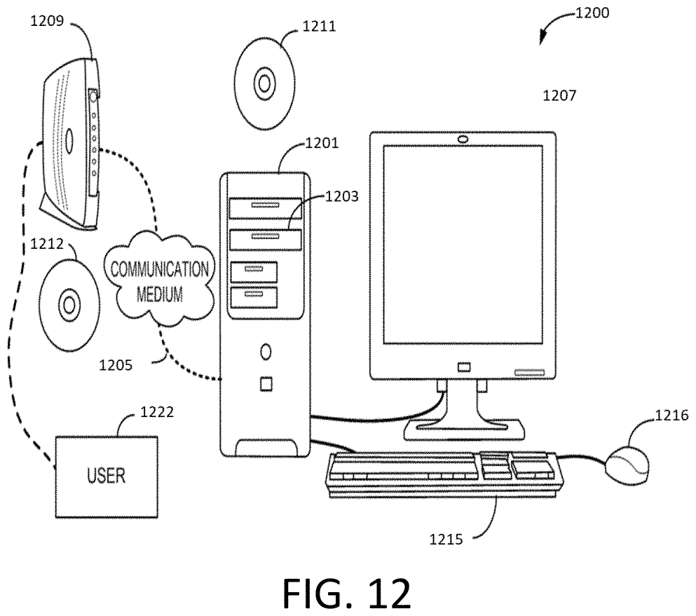

The computer system 1200 illustrated in FIG. 12 may be understood as a logical apparatus that can read instructions from media 1211 and/or a network port 1205, which can optionally be connected to server 1209 having fixed media 1212. The system, such as shown in FIG. 12 can include a CPU 1201, disk drives 1203, optional input devices such as keyboard 1215 and/or mouse 1216 and optional monitor 1207. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 1222 as illustrated in FIG. 12.

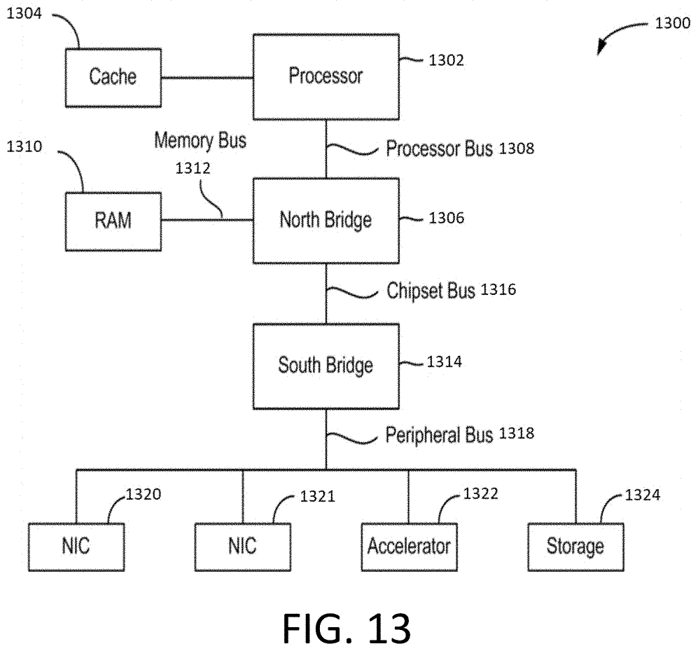

FIG. 13 is a block diagram illustrating a first example architecture of a computer system 1300 that can be used in connection with example instances of the present disclosure. As depicted in FIG. 13, the example computer system can include a processor 1302 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 13, a high speed cache 1304 can be connected to, or incorporated in, the processor 1302 to provide a high speed memory for instructions or data that have been recently, or are frequently, used by processor 1302. The processor 1302 is connected to a north bridge 1306 by a processor bus 1308. The north bridge 1306 is connected to random access memory (RAM) 1310 by a memory bus 1312 and manages access to the RAM 1310 by the processor 1302. The north bridge 1306 is also connected to a south bridge 1314 by a chipset bus 1316. The south bridge 1314 is, in turn, connected to a peripheral bus 1318. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 1318. In some alternative architectures, the functionality of the north bridge can be incorporated into the processor instead of using a separate north bridge chip. In some instances, system 1300 can include an accelerator card 1322 attached to the peripheral bus 1318. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 1324 and can be loaded into RAM 1310 and/or cache 1304 for use by the processor. The system 1300 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present disclosure. In this example, system 1300 also includes network interface cards (NICs) 1320 and 1321 connected to the peripheral bus for providing network interfaces to external storage, such as Network Attached Storage (NAS) and other computer systems that can be used for distributed parallel processing.

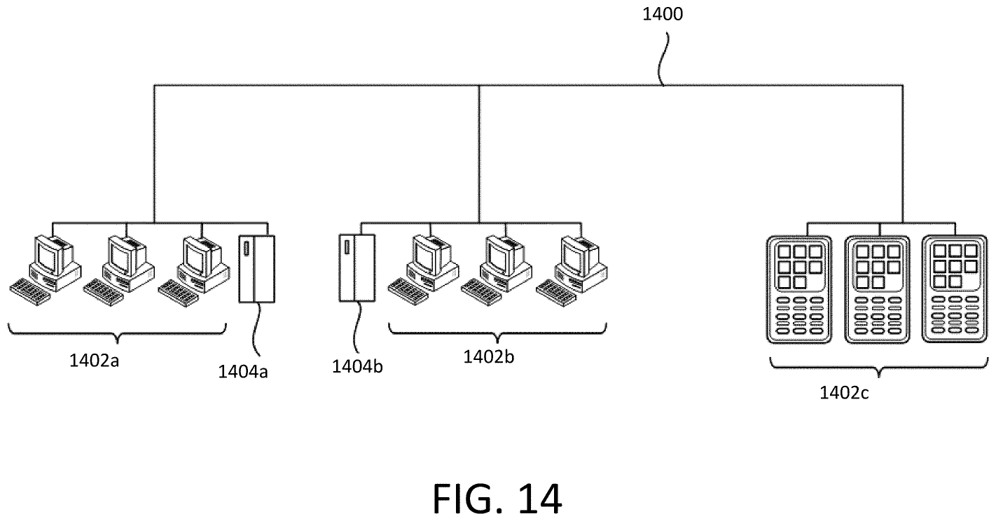

FIG. 14 is a diagram showing a network 1400 with a plurality of computer systems 1402a, and 1402b, a plurality of cell phones and personal data assistants 1402c, and Network Attached Storage (NAS) 1404a, and 1404b. In example instances, systems 1402a, 1402b, and 1402c can manage data storage and optimize data access for data stored in Network Attached Storage (NAS) 1404a and 1404b. A mathematical model can be used for the data and be evaluated using distributed parallel processing across computer systems 1402a, and 1402b, and cell phone and personal data assistant systems 1402c. Computer systems 1402a, and 1402b, and cell phone and personal data assistant systems 1402c can also provide parallel processing for adaptive data restructuring of the data stored in Network Attached Storage (NAS) 1404a and 1404b. FIG. 14 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

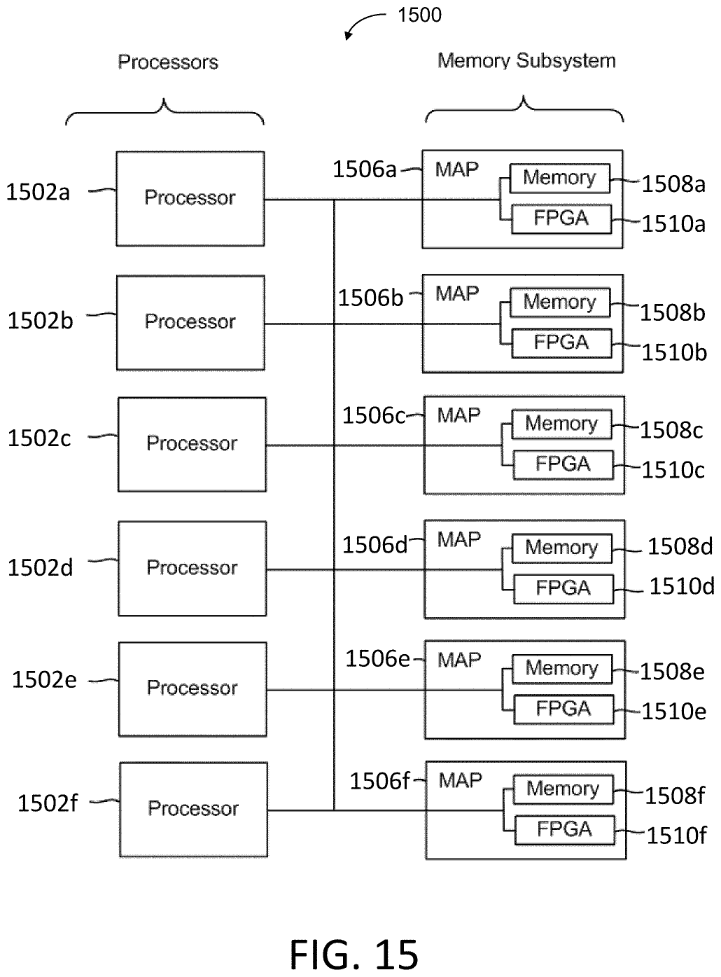

FIG. 15 is a block diagram of a multiprocessor computer system 1500 using a shared virtual address memory space in accordance with an example instance. The system includes a plurality of processors 1502a-f that can access a shared memory subsystem 1504. The system incorporates a plurality of programmable hardware memory algorithm processors (MAPs) 1506a-f in the memory subsystem 1504. Each MAP 1506a-f can comprise a memory 1508a-f and one or more field programmable gate arrays (FPGAs) 1510a-f. The MAP provides a configurable functional unit and particular algorithms or portions of algorithms can be provided to the FPGAs 1510a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 1508a-f, allowing it to execute tasks independently of, and asynchronously from the respective microprocessor 1502a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 15, system on chips (SOCs), application specific integrated circuits (ASICs), or other processing and logic elements. For example, the Set Processor and Optimizer can be implemented with hardware acceleration through the use of a hardware accelerator card, such as accelerator card 1322 illustrated in FIG. 13.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Library Preparation with Universal Adapters

Nucleic acid samples (50 ug) were prepared comprising either dual-index adapters or universal adapters. A ligation master mix is prepared from 20 μL of ligation buffer 10 μL of ligation mix (containing ligase), and 15 μL water. The nucleic acid sample was combined with the ligation mix and incubated at 20 deg C. at 15 minutes. The mixture was then combined with 80 μL of magnetic DNA purification beads, and vortexed, followed by 5 minutes of incubation at room temperature. The mixture was then set on a magnetic plate for 1 min. The beads were then washed with 80% ethanol, incubated for 1 min, and the ethanol wash discarded. The wash was repeated once. Then, beads were air-dried for 5-10 minutes, removed from the magnetic plate, and treated with 17 μL of water, 10 mM Tris-HCl pH 8, or buffer EB. The mixture was homogenized and incubated 2 min at room temperature. The mixture was then placed again on the magnetic plate and incubated 3 min at room temperature, followed by removal of the supernatant containing the universal adapter-ligated genomic DNA. The universal-ligated genomic DNA is combined with 10 μL of barcoded primers and 25 μL of KAPA HiFi HotStart ReadyMix to attach barcodes to the universal primers. The following PCR conditions were used: 1) initialization at 98 deg C. for 45 seconds, 2) a second step comprising: a) denaturation at 98 deg C. for 15 sec, b) annealing at 60 deg C. for 30 sec, and c) extension at 72 deg C. for 30 sec; wherein second step is repeated for 6-8 cycles, 3) final extension at 72 deg C. for 1 minute, and 4) final hold at 4 deg C. Products were purified by DNA beads in a similar manner as previously described. The amplified barcoded library was analyzed on a Qubit dsDNA broad range quantification assay instrument. This library was then sequenced directly. Use of universal adapters resulted in increased library nucleic acid concentration after amplification relative to standard dual-index Y-adapters. The protocol utilizing universal adapters also led to higher total yields after amplification and lower adapter dimer formation. Additionally, a library prepared with universal adapters provided for lower AT dropouts compared to standard dual-index Y-adapters, and resulted in uniform representation of all index sequences. Similarly, universal adapters comprising 10 bp dual indices were utilized (8 PCR cycles, N=12). For comparison, standard full-length Y adapters were also tested for the same genomic DNA sample (10 PCR cycles, N=12).

### Example 2. Library Preparation with Universal Adapters and Enrichment

A nucleic acid sample was prepared using the general methods of Example 1, with modification: dual-index adapters were replaced with universal adapters. After ligation of universal adapters, amplification of the adapter-ligated sample nucleic acid library was conducted with a barcoded primer library, to generate a barcoded adapter-ligated sample nucleic acid library. This library was then subjected to analogous enrichment, purification, and sequencing steps. Use of universal adapters resulted in comparable or better sequencing outcomes.

### Example 3. Library Preparation with Unique Molecular Identifiers

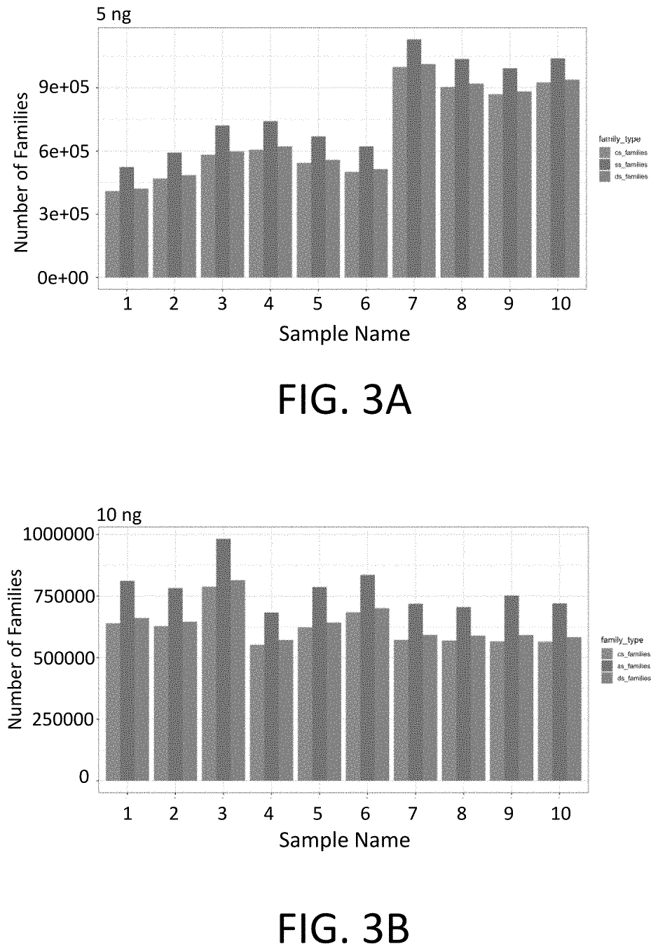

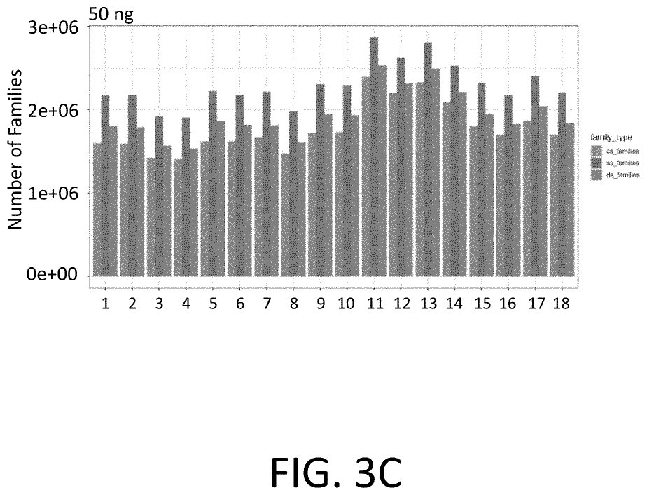

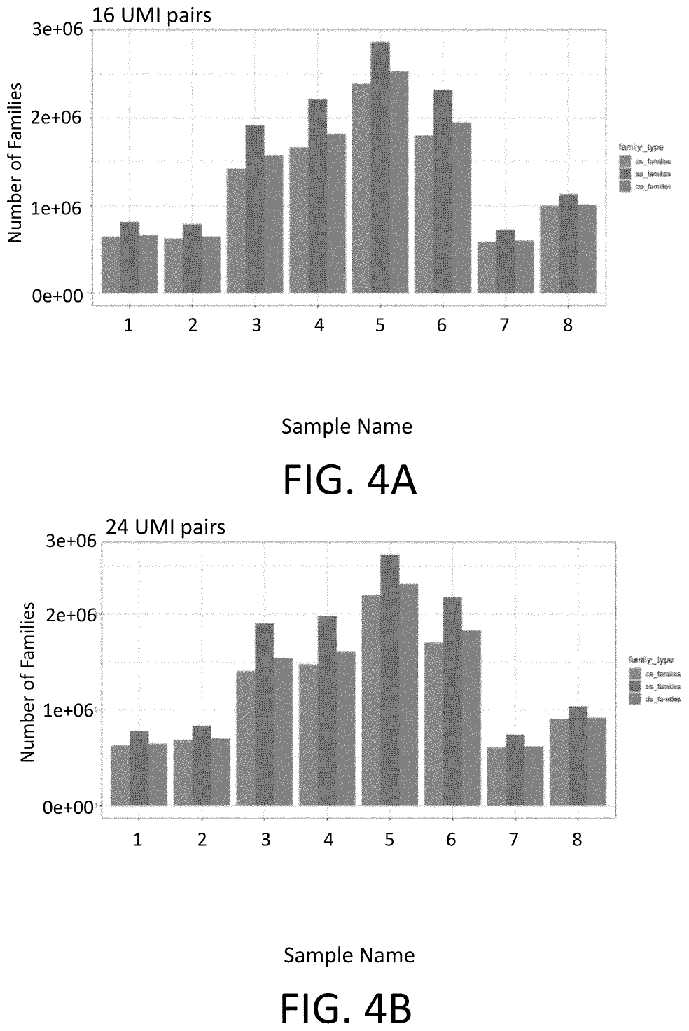

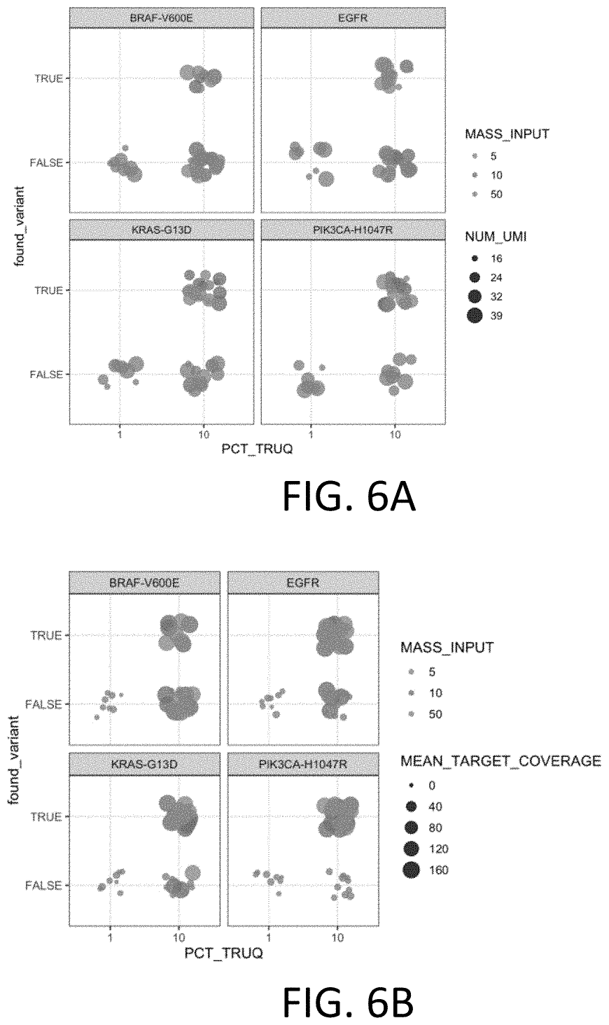

A nucleic acid sample was prepared using the general methods of Example 1, with modification: dual-index adapters comprised a 4-base length UMI, selected from a set of 48 unique sequences having at least a Hamming distance of 2. Adapters were added using the general procedure of FIGS. 1A-1B. Three different amounts of sample genomic DNA were evaluated. A mass titration plot for 5 ng, 10 ng, and 50 ng of input genomic DNA against mean target coverage is shown in FIG. 2B. The number of families was examined for different mass inputs, FIGS. 3A-3C, and the mean target coverage after deduplication increased with increased mass input. The number of families was also examined as a function of different numbers of UMI pairs FIGS. 4A-4B, 5A-5B. Variant calling accuracy was also examined using the TruQ NGS DNA Reference Standard (Table 3). For 10% TruQ vs 1% TruQ, mass input and depth of sequence impacted the accuracy of variant calling (FIGS. 6A-6B).

TABLE 3

| | | | Expected | | |
|---|---|---|---|---|---|
| | | | Allelic | | |
| Chromosome | | | Frequency, | 10% | 1% |
| Number | Gene | Variant | % | dilution | dilution |
| 7p12 | EGFR | G719S | 16.70% | 1.67% | 0.167% |
| 7q34 | BRAF | V600E | 8.00% | 0.8% | 0.08% |
| 12p12.1 | KRAS | G13D | 25.00% | 2.5% | 0.25% |
| 3q26. | PIK3CA | H1047R | 30.00% | 3% | 0.3% |

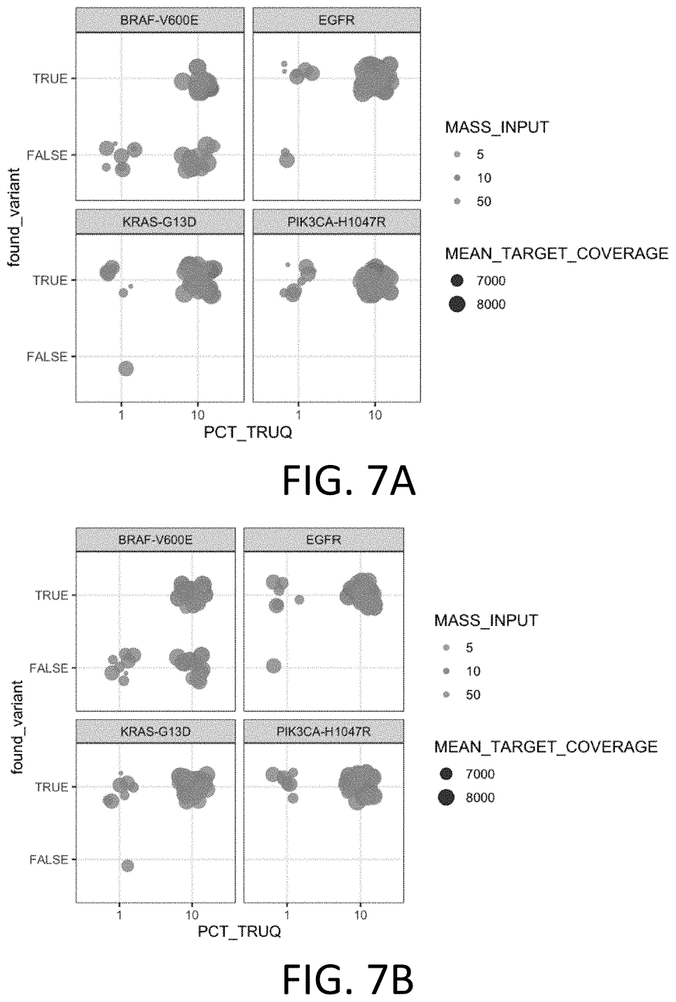

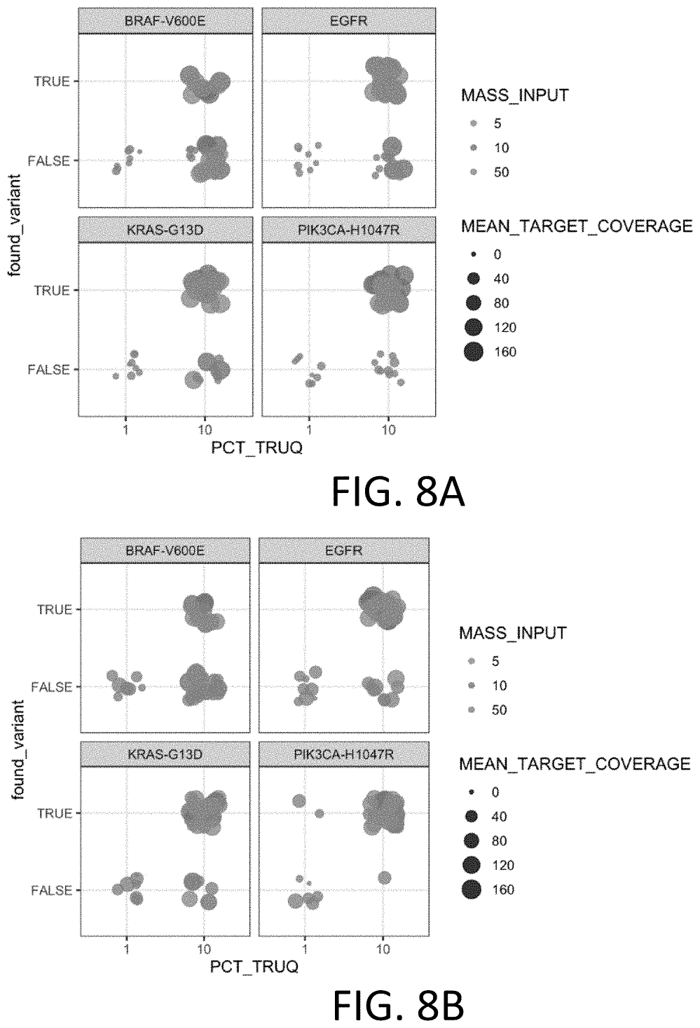

For comparison, variant calling using all reads (no UMIs or deduplication) is shown in FIG. 7A (20,000× read depth), and FIG. 7B (40,000× read depth). Variant calls using duplex consensus reads are shown in FIG. 8A (20,000× read depth), and FIG. 8B (40,000× read depth).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 4. Unique Molecular Identifier Design

Design. A UMI set was designed with the sequences of Table 4.

TABLE 4

| | | Reverse | UMI |
|---|---|---|---|
| | UMI | Complement of | Length |
| Index | Sequence | UMI Sequence | (bp) |
| 1 | AAGGA | TCCTT | 5 |
| 2 | ACAAC | GTTGT | 5 |
| 3 | ATACG | CGTAT | 5 |
| 4 | CACTG | CAGTG | 5 |
| 5 | CATGA | TCATG | 5 |
| 6 | CGATA | TATCG | 5 |
| 7 | CGTGT | ACACG | 5 |
| 8 | GCCAT | ATGGC | 5 |
| 9 | GCTGT | ACAGC | 5 |
| 10 | GTCAC | GTGAC | 5 |
| 11 | GTCGT | ACGAC | 5 |
| 12 | TACGA | TCGTA | 5 |
| 13 | TCCTA | TAGGA | 5 |
| 14 | TCGTG | CACGA | 5 |
| 15 | TGTCG | CGACA | 5 |
| 16 | TTGGC | GCCAA | 5 |
| 17 | AACAC | GTGTT | 5 |
| 18 | AATGC | GCATT | 5 |
| 19 | ACTAG | CTAGT | 5 |
| 20 | AGCAT | ATGCT | 5 |
| 21 | AGTAC | GTACT | 5 |
| 22 | ATCTC | GAGAT | 5 |
| 23 | CAGAC | GTCTG | 5 |
| 24 | CAGTA | TACTG | 5 |
| 25 | CGAAT | ATTCG | 5 |
| 26 | CGGTT | AACCG | 5 |
| 27 | CTTGG | CCAAG | 5 |
| 28 | GCATA | TATGC | 5 |
| 29 | GCTAA | TTAGC | 5 |
| 30 | GTGAG | CTCAC | 5 |
| 31 | GTGTC | GACAC | 5 |
| 32 | TGTGC | GCACA | 5 |

Low-input sample sources like liquid biopsy are increasingly common in cancer diagnostics and monitoring. Sequencing analysis of these sample types requires robust methods to confidently detect low frequency alleles. In these cases, unique molecular identifiers (UMIs) are a valuable addition to the standard NGS workflow. UMIs offer increased sensitivity in quantitative variant identification where the data may be susceptible to confounding PCR duplicates. Additionally, mechanical Fragmentation may be used to produce sequencing-ready libraries. The 5 basepair matched UMI adapters are first ligated to the input molecules, and subsequently PCR amplified with Unique Dual-Indexed primers. The resulting indexed libraries enable post-sequencing consensus building, which offers better detection sensitivity compared to standard methods.

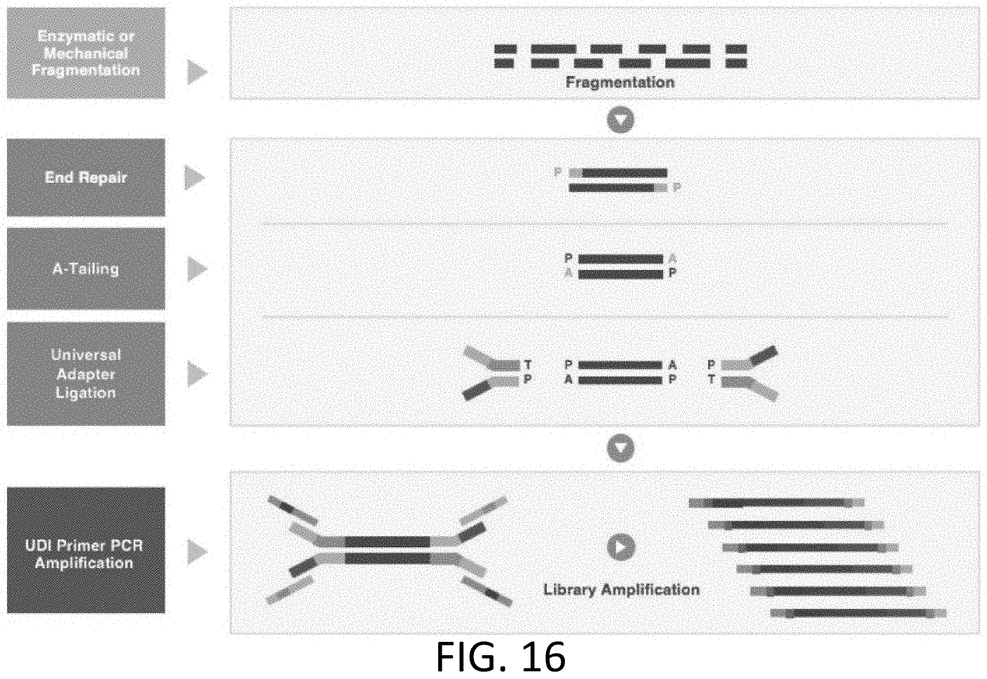

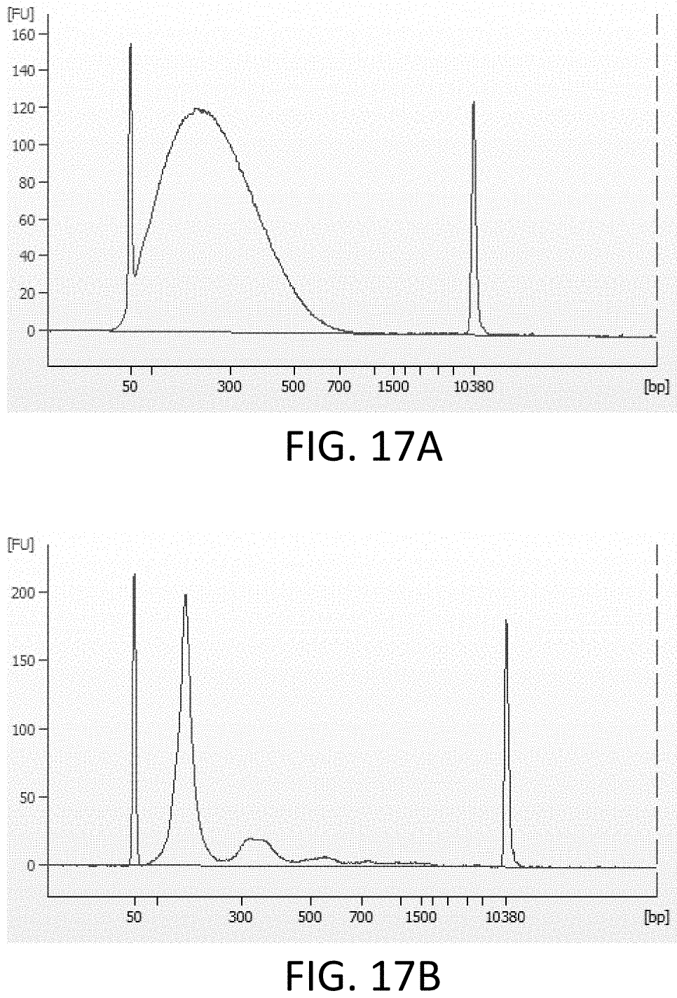

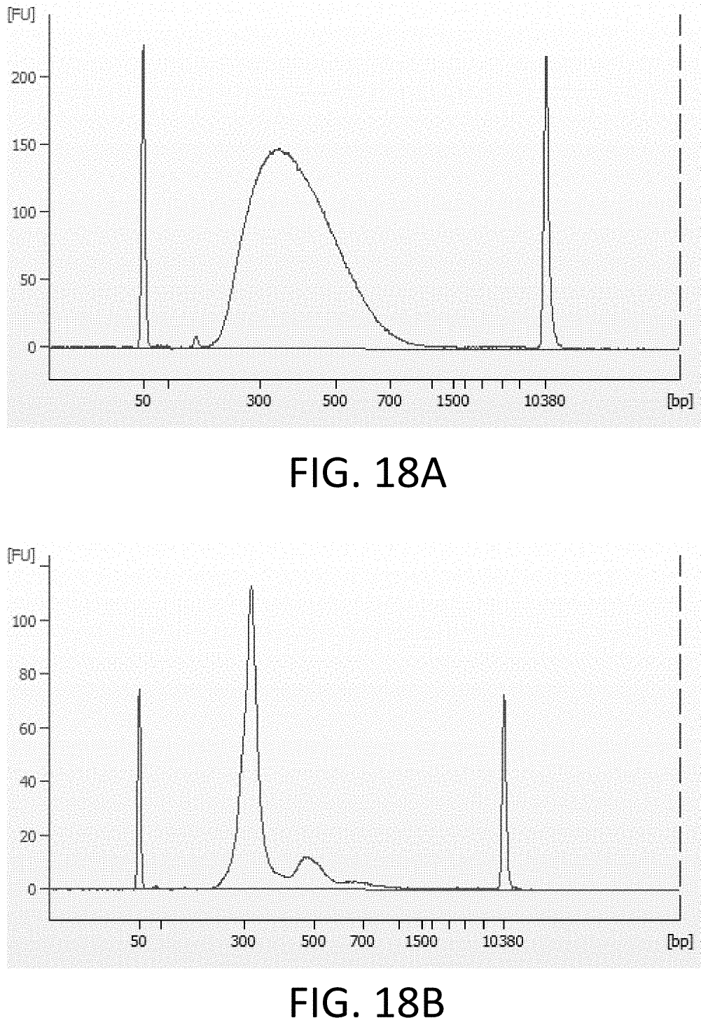

Methods. UMIs were included into adapters using the general design shown in FIG. 1A, and incorporated into a library sequencing workflow as shown in FIG. 16 and general methods of Example 1. Samples were first analyzed after mechanical fragmentation of genomic DNA (FIG. 17A) or from cfDNA (FIG. 17B). Adapters comprising 5-base UMIs were then added and the library amplified to add 10 bp dual indexes (DNA (FIG. 18A) or cfDNA (FIG. 18B)). Optionally, libraries that can be further enriched with custom or fixed target enrichment panels.

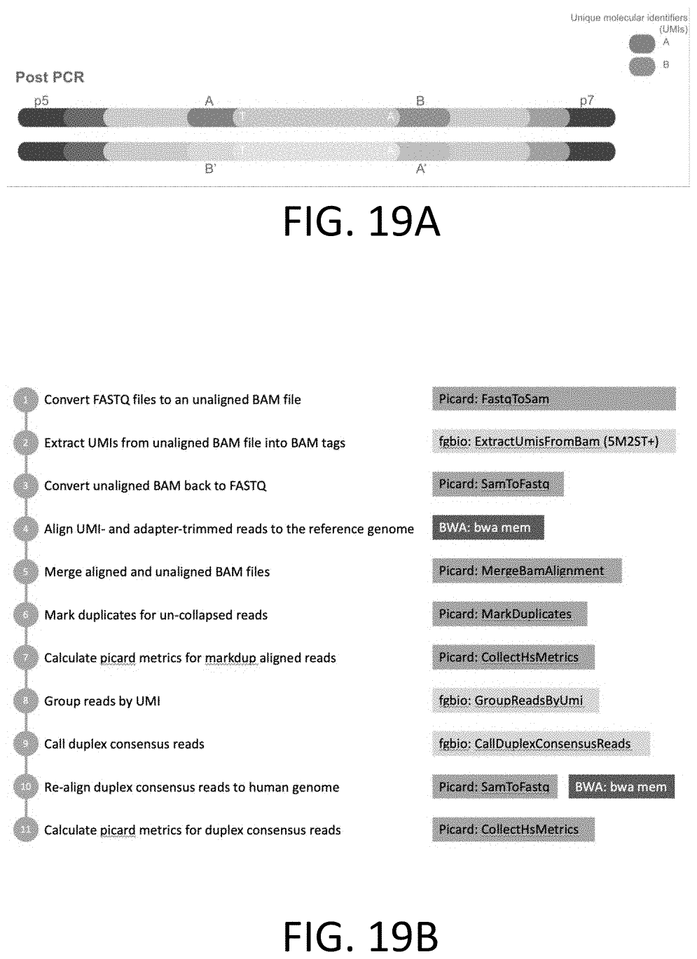

Processing of UMI data. Consensus reads were called from UMI families (general structure depicted in FIG. 19A) using the workflow in FIG. 19B. During post processing of sequencing data to identify UMI families, a minimum of 2 bp was trimmed between the 5 bp UMI sequence and the rest of the read to ensure proper UMI family identification. The pipeline for calling utilized fgbio, an open source command line toolkit from Fulcrum Genomics. The BWA and Picard tools software packages were also used. FASTQ files were first converted to unaligned BAM to extract the in-line UMI sequences, which were then stored in a BAM tag. Unaligned BAM files were then converted back to FASTQ for alignment to the reference genome. The resulting aligned BAM files were then merged with unaligned BAM files containing the UMI tag, which were processed by the fgbio suite to create duplex consensus reads. Additionally:
1. Used GroupReadsByUmi to group reads together that come from the same original molecule. This groups reads by mapping position (chromosome, start and end) as well as by UMI sequence.
2. Used CaliDuplexConsensusReads to create duplex consensus reads by combining the evidence from a top and bottom single stranded consensus read.
   The final consensus BAM was then be used for tertiary data analysis. For the ExtractUmisFromBam tool, the read structure for libraries prepared was 5M2S144T for PE151 sequencing, where M=UMI sequence (5 bp), S=shadow/skipped bases (2 bp), and T=target/DNA bases (144 bp).

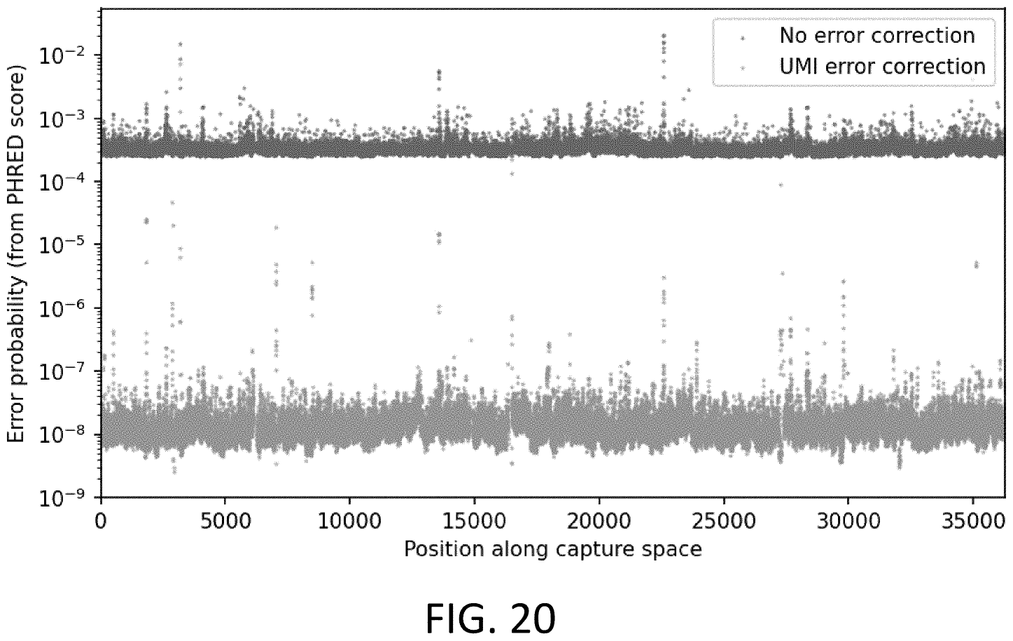

Results with error correction with and without UMIs is shown in FIG. 20. Estimated per-base error rate non-error-corrected reads (top) and error-corrected reads (bottom) measured from target-enrichment of a synthetic cfDNA standard. After sequencing, reads were downsampled to 20,000× coverage and error correction was performed using duplex collapse, requiring at least one read from each original strand.

1.-45. (canceled)

46. A method of sequencing comprising:
(a) ligating one or more polynucleotide adapters to a plurality of sample nucleic acids to generate a library of adapter-ligated sample polynucleotides, wherein at least some of the polynucleotide adapters comprise a unique molecular identifier, wherein the library comprises a plurality of unique molecular identifiers;
(b) amplifying the library;
(c) sequencing the library to generate a plurality of reads;
(d) trimming one or more bases in each of the plurality of reads, wherein trimming comprises removal of one or more bases from one or both of:
(i) a unique molecular identifier sequence; or
(ii) a sequence between the unique molecular identifier sequence and a sample nucleic acid sequence; and
(e) organizing the plurality of reads based on the plurality of unique molecular identifiers to distinguish between amplification errors and single nucleotide polymorphisms present in the sample nucleic acids.

47. The method of claim 46, wherein each of the at least some of the polynucleotide adapters comprises a first molecular identifier and a second molecular identifier.

48. The method of claim 46, wherein the library comprises at least 8 different unique molecular identifiers.

49. The method of claim 47, wherein the first unique molecular identifier and the second unique molecular identifier are selected from a set of no more than 64 sequences.

50. The method of claim 47, wherein the first unique molecular identifier and the second unique molecular identifier are selected from a set of no more than 48 sequences.

51. The method of claim 46, wherein each of the plurality of polynucleotide adapters comprises:
(i) a first strand, wherein the first strand comprises a first terminal adapter region, a first non-complementary region, a first yoke region, and the first unique molecular identifier; and
(ii) a second strand, wherein the second strand comprises a second terminal adapter region, a second non-complementary region, a second yoke region, and the second unique molecular identifier.

52. The method of claim 51, wherein the first yoke region and the second yoke region are complementary and wherein the first non-complementary region and the second non-complementary region are not complementary.

53. The method of claim 51, wherein the first yoke region and the second yoke region are each less than 15 bases in length.

54. The method of claim 47, wherein the first unique molecular identifier and the second unique molecular identifier are complementary.

55. The method of claim 47, wherein the first unique molecular identifier or the second unique molecular identifier comprise the sequences of one or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACACA, AATGCC, ACTAGG, AGCATC, AGTACA, ATCTCC, CAGACG, CAGTAC, CGAATC, CGGTTG, CTTGGA, GCATAG, GCTAAC, GTGAGA, GTGTCA, and TGTGCC.

56. The method of claim 47, wherein the first unique molecular identifier or the second unique molecular identifier comprise the sequences of 10 or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACACA, AATGCC, ACTAGG, AGCATC, AGTACA, ATCTCC, CAGACG, CAGTAC, CGAATC, CGGTTG, CTTGGA, GCATAG, GCTAAC, GTGAGA, GTGTCA, and TGTGCC.

57. The method of claim 47, wherein the first unique molecular identifier or the second unique molecular identifier comprise the sequences of one or more of AAGGA, ACAAC, ATACG, CACTG, CATGA, CGATA, CGTGT, GCCAT, GCTGT, GTCAC, GTCGT, TACGA, TCCTA, TCGTG, TGTCG, and TTGGC.

58. The method of claim 47, wherein the first unique molecular identifier or the second unique molecular identifier comprise the sequences of one or more of AACACA, AATGCC, ACTAGG, AGCATC, AGTACA, ATCTCC, CAGACG, CAGTAC, CGAATC, CGGTTG, CTTGGA, GCATAG, GCTAAC, GTGAGA, GTGTCA, and TGTGCC

59. The method of claim 47, wherein the first unique molecular identifier and the second unique molecular identifier are selected from a set of sequences having a Hamming distance of at least 2.

60. The method of claim 46, wherein the sample nucleic acid is genomic DNA.

61. The method of claim 46, wherein organizing the plurality of reads comprises:
(i) extracting sequences of the plurality of unique molecular identifiers from unaligned reads, wherein each of the sequences is stored as a tag;
(ii) aligning the unaligned reads to a reference genome to generate aligned reads; and
(iii) merging the aligned reads with the unaligned reads comprising the tags to generate consensus reads.

62. The method of claim 61, wherein the sequences comprise one or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC.

63. The method of claim 61, wherein the sequences comprise 10 or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC.

64. The method of claim 61, wherein the sequences comprise one or more of AAGGA, ACAAC, ATACG, CACTG, CATGA, CGATA, CGTGT, GCCAT, GCTGT, GTCAC, GTCGT, TACGA, TCCTA, TCGTG, TGTCG, and TTGGC.

65. The method of claim 61, wherein the sequences comprise one or more of AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC.
